(19) United States
(12) Patent Application Publication
(10) Pub. No.: US 2024/0216612 A1
(43) Pub. Date: Jul. 4, 2024

(54) DUAL CHAMBER INJECTION SYSTEM AND METHODS OF USE THEREOF

(71) Applicant: Dali Medical Devices LTD., Yavne (IL)

(72) Inventors: David DAILY, Herzliya (IL); Gad LEWKONYA, Neve Mivtach (IL); Lior RADAY, Kibbutz Bror-Hail (IL); Hagay Drori, Tel-Aviv (IL); Dmitri SARKOROV, Rosh HaAyin (IL); Guy Keenan, Tel Aviv (IL); Ziv Cahani, Ramat-Hasharon (IL); Sasha Leikin, Tel-Aviv (IL); Amir Loval, Tel-Aviv (IL); Omri Bar Zeev, Tel-Aviv (IL)

(21) Appl. No.: 18/558,547

(22) PCT Filed: May 11, 2022
(86) PCT No.: PCT/IL2022/050488

§ 371 Date: Nov. 2, 2023

Related U.S. Application Data
(60) Provisional application No. 63/187,977, filed on May 13, 2021.

(51) Int. Cl. A61M 5/20; A61M 5/315

(52) U.S. Cl. CPC A61M 5/2066; A61M 5/31501; A61M 2005/31508

(57) ABSTRACT

A dual chamber injection system, comprising a guiding element arranged along a longitudinal axis and configured to at least partially contain a medicament container, the guiding element defines an inner surface and at least one protrusion extending radially inwardly therefrom; a plunger rod assembly, comprising a plunger outer portion arranged along the longitudinal axis and a plunger inner portion arranged concentrically within the plunger outer portion and having at least one guiding groove arranged on an outer surface of the plunger inner portion; the plunger outer portion is disposed externally relative to the guiding element; the plunger inner portion is at least partially received into the guiding element; and wherein the plunger rod assembly is displaceable relative to the guiding element and the displacement is guided by engagement of the at least one protrusion with the at least one guiding groove.

### REFERENCE TO RELATED APPLICATIONS

Reference is hereby made to U.S. Provisional Patent Application Ser. No. US 63/187,977, filed and entitled “DUAL CHAMBER INJECTION SYSTEM AND METHODS OF USE THEREOF”, the disclosure of which is hereby incorporated by reference in its entirety and priority of which is hereby claimed.

Reference is hereby made to U.S. patent application Ser. No. 16/994,596, filed Aug. 16, 2020 and entitled “Dual Chamber Syringe and Methods of Use Thereof” and to U.S. Provisional Patent Application No. 62/911,383, filed Oct. 7, 2019 and entitled “Dual Chamber Injection System and Methods of Use Thereof”, the disclosures of which are hereby incorporated by reference in their entirety.

### FIELD OF THE INVENTION

The present invention generally relates to a dual chamber injection system, and more specifically to a pre-fillable dual chamber injection system and methods of use thereof for reconstitution and injection of a medicament.

### BACKGROUND OF THE INVENTION

Pre-fillable dual chamber injection systems are known in the art for separately containing a powder/liquid medicament preparation and a solvent in different chambers of the syringe.

It is also known that pre-fillable dual chamber injection systems preferably include a syringe barrel with several pistons, which are slidably sealingly disposed therewithin and divide the syringe barrel into several separate chambers, whereas one of the chambers contains a powder medicament and another contains a solvent. Alternatively, both chambers may include liquids that are stored separately and that shall be mixed only at the time of injection.

The pre-fillable dual chamber injection systems also include a plunger rod, which is operative to engage one of the pistons. Upon displacement of the plunger rod relative to the syringe barrel, the pistons are advanced within the syringe barrel and permit reconstitution of the medicament by mixing the powder medicament with the solvent using a bypass formed in the syringe barrel.

### SUMMARY OF THE INVENTION

The present invention seeks to provide a dual chamber injection system.

There is thus provided in accordance with an embodiment of the present invention or a combination of embodiments thereof a dual chamber injection system, comprising a guiding element arranged along a longitudinal axis and configured to at least partially contain a medicament container, the guiding element defines an inner surface and at least one protrusion extending radially inwardly therefrom; a plunger rod assembly, comprising a plunger outer portion arranged along the longitudinal axis and a plunger inner portion arranged concentrically within the plunger outer portion and having at least one guiding groove arranged on an outer surface of the plunger inner portion; the plunger outer portion is disposed externally relative to the guiding element; the plunger inner portion is at least partially received into the guiding element; and wherein the plunger rod assembly is displaceable relative to the guiding element and the displacement is guided by engagement of the at least one protrusion with the at least one guiding groove.

Preferably, the guiding element defines an outer surface and at least one arm extending therefrom and being radially deflectable upon application of pressure thereon; the plunger outer portion having a plurality of openings formed along the circumference thereof; and wherein an indication is provided to the user to indicate transitions between different operative orientations of the dual chamber injection system provided by engagement of the at least one arm with one of the plurality of openings.

Further preferably, the at least one guiding groove is continuous and having portions thereof that are disposed in different axial orientations along the outer surface of said plunger inner portion. Yet, further preferably, the at least one guiding groove is continuous and having portions thereof that are disposed in different angular orientations along the outer surface of the plunger inner portion.

Still further preferably, in a locked operative orientation of the dual chamber injection system the plunger rod assembly is prevented from axial displacement relative to the guiding element due to engagement of the at least one protrusion with a locking portion of the at least one guiding groove. Yet further preferably, the guiding element further comprises at least one locking arm; and the outer plunger portion has a rearwardly facing surface formed on the inner surface; and wherein in an end of injection operative orientation of the dual chamber injection system the plunger rod assembly is prevented from rearward displacement relative to the guiding element due to engagement of the at least one locking arm with the rearwardly facing surface. Still further preferably, the indication is either a tactile indication or an audible indication.

In accordance with an embodiment of the present invention, the dual chamber injection system also comprising an outer housing configured to enclose the guiding element therewithin and being fixedly attached thereto, and wherein the outer hosing has a plurality of openings and demarcations provided on an outer surface thereof.

Preferably, a visual indication of different operative orientations of the dual chamber injection system is provided to a user by alignment of at least one of the openings and demarcations of the outer housing with corresponding demarcations formed on an outer surface of the plunger outer portion.

Further preferably, rearward axial displacement of the plunger rod assembly relative to the guiding element is prevented due to engagement of the at least one arm of the guiding element with the one of the plurality of openings of the plunger outer portion.

Still further preferably, the plunger rod assembly is rotatable relative to the guiding element in some operative orientations when the at least one protrusion of the guiding element engages a certain portion of the at least one guiding groove, and wherein the plunger rod assembly is axially displaceable relative to the guiding element in other operative orientations when the at least one protrusion of the guiding element engages another portion of the at least one guiding groove.

In accordance with an embodiment of the present invention, a dual chamber injection system, comprising a guiding element arranged along a longitudinal axis and configured to at least partially contain a medicament container, the guiding element defines an outer surface and at least one arm extending therefrom and being radially deflectable upon application of pressure thereon; a plunger rod assembly, comprising a plunger outer portion arranged along the longitudinal axis and a plunger inner portion arranged concentrically within the plunger outer portion; the plunger outer portion having a plurality of openings formed along the circumference thereof; the plunger outer portion is disposed externally relative to the guiding element; the plunger inner portion is at least partially received into the guiding element; and wherein an indication is provided to the user to indicate transitions between different operative orientations of the dual chamber injection system provided by engagement of the at least one arm with one of the plurality of openings.

Preferably, the guiding element defines an inner surface and at least one protrusion extending radially inwardly therefrom; the plunger inner portion having at least one guiding groove arranged on an outer surface of the plunger inner portion; and wherein the plunger rod assembly is displaceable relative to the guiding element and the displacement is guided by engagement of the at least one protrusion with the at least one guiding groove.

Further preferably, the at least one guiding groove is continuous and having portions thereof that are disposed in different axial and angular orientations along the outer surface of the plunger inner portion. Still further preferably, in a locked operative orientation of the dual chamber injection system the plunger rod assembly is prevented from axial displacement relative to the guiding element due to engagement of the at least one protrusion with a locking portion of the at least one guiding groove. Yet further preferably, the guiding element further comprises at least one locking arm; and the outer plunger portion has a rearwardly facing surface formed on the inner surface; and wherein in an end of injection operative orientation of the dual chamber injection system the plunger rod assembly is prevented from forward displacement relative to the guiding element due to engagement of the at least one locking arm with the rearwardly facing surface. Still further preferably, the indication is either a tactile indication or an audible indication.

In accordance with an embodiment of the present invention, the dual chamber injection system also comprising an outer housing configured to enclose the guiding element therewithin and being fixedly attached thereto, and wherein the outer hosing has a plurality of openings and demarcations provided on an outer surface thereof.

Preferably, a visual indication of different operative orientations of the dual chamber injection system is provided to a user by alignment of at least one of the openings and demarcations of the outer housing with corresponding demarcations formed on an outer surface of the plunger outer portion.

Further preferably, rearward axial displacement of the plunger rod assembly relative to the guiding element is prevented due to engagement of the at least one arm of the guiding element with the one of the plurality of openings of the plunger outer portion. Yet further preferably, rotational displacement of the plunger rod assembly relative to the guiding element is prevented in at least one rotational direction due to engagement of the at least one arm of the guiding element with the one of the plurality of openings of the plunger outer portion.

Still further preferably, the plunger rod assembly is rotatable relative to the guiding element in some operative orientations when the at least one protrusion of the guiding element engages a certain portion of the at least one guiding groove, and wherein the plunger rod assembly is axially displaceable relative to the guiding element in other operative orientations when the at least one protrusion of the guiding element engages another portion of the at least one guiding groove.

In accordance with an embodiment of the present invention, the dual chamber system, comprising a housing element arranged along a longitudinal axis and configured to at least partially contain a medicament container; a plunger rod assembly, comprising a plunger outer portion arranged along the longitudinal axis and a plunger inner portion arranged concentrically within the plunger outer portion and having at least one guiding groove arranged on the plunger outer portion; the plunger rod assembly is at least partially received into the housing element; a ring assembly mounted onto a portion of the housing element and having a radially inwardly extending protrusion engageable with a portion of the at least one guiding groove, whereas displacement of the radially inwardly extending protrusion along the guiding groove enables transition of the dual chamber injection system between the different operative orientations thereof.

Preferably, the dual chamber injection system also comprising a guiding element being at least partially received into the housing element and configured to at least partially contain the medicament container having a septum at a forward end thereof.

Further preferably, the different operative orientations comprising locked orientation, septum penetration, medicament reconstitution, priming and medicament injection. Still further preferably, the ring assembly is being rotatable and engageable with the plunger outer portion, whereas axial displacement of the plunger rod assembly relative to the housing element is permitted upon rotation of the ring assembly relative to the housing element, thereby enabling transition of the dual chamber injection system between at least some of the different operative orientations thereof.

Yet further preferably, rotation of the ring assembly relative to the housing element results in alignment of corresponding indication features disposed on the housing element and the ring assembly, thereby enables indicating transition between the different operative orientations to the user.

In accordance with an embodiment of the present invention, the axial displacement of the plunger assembly relative to the housing element results in displacement of at least one of the pistons contained within the medicament container, thereby enabling at least one of reconstitution of medicament, priming of the medicament container and injection of medicament.

Preferably, the at least one guiding groove comprises a locking portion, a reconstitution portion, a helical priming portion and an injection portion, wherein the reconstitution portion is radially and axially offset from the injection portion and the locking portion is disposed generally transversely with respect to the reconstitution portion.

Further preferably, a needle is fixedly attached to the housing element and the medicament container is rearwardly spaced from the needle prior to initial forward axial displacement of the plunger assembly, such that the needle does not penetrate the septum. Still further preferably, the guiding element has a snap portion that is operatively engaged to the plunger inner portion for preventing rearward displacement of the plunger rod assembly relative to the housing element. Yet further preferably, forward axial displacement of the plunger rod assembly relative to the housing element is prevented due to engagement of the ring assembly with the guiding groove in a locked operative orientation of the injection system.

In accordance with an embodiment of the present invention, the ring assembly has a radially inwardly extending snap portion, which allows rotation of the ring assembly relative to the housing element in a first rotational direction and prevents rotation of the ring assembly in a second rotational direction, which is opposite to the first rotational direction.

Preferably, relative axial displacement between the plunger rod assembly and the guiding element is prevented in a locked operative orientation of the injection system, before initial axial displacement of the plunger assembly. Further preferably, relative rotation is prevented between the housing element, the guiding element and the plunger rod assembly in all operative orientations of the dual chamber injection system. Still further preferably, the plunger rod assembly in a priming operative orientation is caused to be displaced axially forwardly due to thread-like engagement of the radially inwardly extending protrusion of the ring assembly with a helical priming portion of the guiding groove.

Yet further preferably, initially, the plunger rod assembly being engaged with the guiding element, such that axial displacement of the plunger rod assembly relative to the housing element results in axial displacement of the guiding element relative to the housing element up to penetration of the septum by the needle; and subsequently the plunger rod assembly being disengaged from the guiding element due to further axial displacement of the plunger rod assembly relative to the guiding element, which does not result in further axial displacement of the guiding element relative to the housing element.

Preferably, a needle is configured to be fixedly attached to the housing element and a protective cap removably attached to the housing element and configured to protect the needle in certain operative orientations of the dual chamber injection system; and wherein the plunger rod assembly is prevented from forward axial displacement relative to the housing element beyond a certain point up to removal of the protective cap from the housing element.

In accordance with an embodiment of the present invention, a dual chamber injection system, comprising: a housing element arranged along a longitudinal axis and configured to at least partially contain a medicament container; a plunger rod assembly, comprising a plunger outer portion arranged along the longitudinal axis and a plunger inner portion arranged concentrically within the plunger outer portion; the plunger outer portion is at least partially received into the housing element; a rotatable ring assembly mounted onto a portion of the housing element and engageable with the plunger outer portion, whereas axial displacement of the plunger rod assembly relative to the housing element is permitted upon rotation of the ring assembly relative to the housing element, thereby enabling transition of the dual chamber injection system between at least some of the different operative orientations thereof.

Preferably, at least one guiding groove is arranged on the plunger outer portion; a radially inwardly extending protrusion is formed on the ring assembly and is being engageable with a portion of the at least one guiding groove, whereas displacement of the radially inwardly extending protrusion along the guiding groove enables transition of the dual chamber injection system between the different operative orientations thereof.

Further preferably, the dual chamber injection system also comprising a guiding element being at least partially received into the housing element and configured to at least partially contain the medicament container having a septum at a forward end thereof. Still further preferably, the different operative orientations comprising locked orientation, septum penetration, medicament reconstitution, priming and medicament injection. Yet further preferably, rotation of the ring assembly relative to the housing element results in alignment of corresponding indication features disposed on the housing element and the ring assembly, thereby enables indicating transition between the different operative orientations to the user.

In accordance with an embodiment of the present invention, the axial displacement of the plunger assembly relative to the housing element results in displacement of at least one of the pistons contained within the medicament container, thereby enabling at least one of reconstitution of medicament, priming of the medicament container and injection of medicament.

Preferably, the at least one guiding groove comprises a locking portion, a reconstitution portion, a helical priming portion and an injection portion, wherein the reconstitution portion is radially and axially offset from the injection portion and the locking portion is disposed generally transversely with respect to the reconstitution portion. Further preferably, a needle is fixedly attached to the housing element and the medicament container is rearwardly spaced from the needle prior to initial forward axial displacement of the plunger assembly, such that the needle does not penetrate a septum of the medicament container.

Still further preferably, the guiding element has a snap portion that is operatively engaged to the plunger inner portion for preventing rearward displacement of the plunger rod assembly relative to the housing element. Yet further preferably, forward axial displacement of the plunger rod assembly relative to the housing element is prevented due to engagement of the ring assembly with the guiding groove in a locked operative orientation of the injection system.

In accordance with an embodiment of the present invention, the ring assembly has a radially inwardly extending snap portion, which allows rotation of the ring assembly relative to the housing element in a first rotational direction and prevents rotation of the ring assembly in a second rotational direction, which is opposite to the first rotational direction.

Preferably, relative axial displacement between the plunger rod assembly and the guiding element is prevented in a locked operative orientation of the injection system, before initial axial displacement of the plunger assembly. Further preferably, relative rotation is prevented between the housing element, the guiding element and the plunger rod assembly in all operative orientations of the dual chamber injection system.

Still further preferably, the plunger rod assembly in a priming operative orientation is caused to be displaced axially forwardly due to thread-like engagement of the radially inwardly extending protrusion of the ring assembly with a helical priming portion of the guiding groove. Yet further preferably, initially, the plunger rod assembly being engaged with the guiding element, such that axial displacement of the plunger rod assembly relative to the housing element results in axial displacement of the guiding element relative to the housing element up to penetration of the septum by a needle, which is configured to be attached to the housing element; and subsequently the plunger rod assembly being disengaged from the guiding element due to further axial displacement of the plunger rod assembly relative to the guiding element, which does not result in further axial displacement of the guiding element relative to the housing element.

Preferably, a needle is configured to be fixedly attached to the housing element and a protective cap removably attached to the housing element and configured to protect the needle in certain operative orientations of the dual chamber injection system; and wherein the plunger rod assembly is prevented from forward axial displacement relative to the housing element beyond a certain point up to removal of the protective cap from the housing element.

In accordance with an embodiment of the present invention, a dual chamber injection system, comprising a housing element arranged along a longitudinal axis, having a needle fixedly attached thereto; a guiding element being at least partially received into the housing element and configured to at least partially contain a medicament container having a septum at a forward end thereof; a plunger rod assembly at least partially received into the housing element; initially, the plunger rod assembly being engaged with the guiding element, such that axial displacement of the plunger rod assembly relative to the housing element results in axial displacement of the guiding element relative to the housing element up to penetration of the septum by the needle; and subsequently the plunger rod assembly being disengaged from the guiding element due to further axial displacement of the plunger rod assembly relative to the guiding element, which does not result in further axial displacement of the guiding element relative to the housing element.

Preferably, the plunger rod assembly, comprising a plunger outer portion arranged along the longitudinal axis and a plunger inner portion arranged concentrically within the plunger outer portion and having at least one guiding groove arranged on the plunger outer portion; the plunger rod assembly is at least partially received into the housing element; a ring assembly mounted onto a portion of the housing element and having a radially inwardly extending protrusion engageable with a portion of the at least one guiding groove, whereas displacement of the radially inwardly extending protrusion along the guiding groove enables transition of the dual chamber injection system between the different operative orientations thereof.

Further preferably, the different operative orientations comprising locked orientation, septum penetration, medicament reconstitution, priming and medicament injection. Still further preferably, the ring assembly is being rotatable and engageable with the plunger outer portion, whereas axial displacement of the plunger rod assembly relative to the housing element is permitted upon rotation of the ring assembly relative to the housing element, thereby enabling transition of the dual chamber injection system between at least some of the different operative orientations thereof.

Yet further preferably, rotation of the ring assembly relative to the housing element results in alignment of corresponding indication features disposed on the housing element and the ring assembly, thereby enables indicating transition between the different operative orientations to the user.

In accordance with an embodiment of the present invention, the axial displacement of the plunger assembly relative to the housing element results in displacement of at least one of the pistons contained within the medicament container, thereby enabling at least one of reconstitution of medicament, priming of the medicament container and injection of medicament.

Preferably, forward axial displacement of the plunger rod assembly relative to the housing element is prevented due to engagement of the ring assembly with the guiding groove in a locked operative orientation of the injection system. Further preferably, the ring assembly has a radially inwardly extending snap portion, which allows rotation of the ring assembly relative to the housing element in a first rotational direction and prevents rotation of the ring assembly in a second rotational direction, which is opposite to the first rotational direction.

Still further preferably, relative axial displacement between the plunger rod assembly and the guiding element is prevented in a locked operative orientation of the injection system, before initial axial displacement of the plunger assembly. Yet further preferably, the plunger rod assembly in a priming operative orientation is caused to be displaced axially forwardly due to thread-like engagement of the radially inwardly extending protrusion of the ring assembly with a helical priming portion of the guiding groove.

Preferably, a protective cap being removably attached to the housing element and configured to protect the needle in certain operative orientations of the dual chamber injection system; and wherein the plunger rod assembly is prevented from forward axial displacement relative to the housing element beyond a certain point up to removal of the protective cap from the housing element.

In accordance with an embodiment of the present invention, a dual chamber injection system, comprising a housing element arranged along a longitudinal axis, having a needle fixedly attached thereto; a protective cap removably attached to the housing and configured to protect the needle in certain operative orientations of the dual chamber injection system; a plunger rod assembly at least partially received into the housing element; wherein the plunger rod assembly is prevented from forward axial displacement relative to the housing element beyond a certain point up to removal of the protective cap from the housing element.

Preferably, the dual chamber injection system also comprising a guiding element being at least partially received into the housing element and configured to at least partially contain a medicament container having a septum at a forward end thereof; wherein initially, the plunger rod assembly being engaged with the guiding element, such that axial displacement of the plunger rod assembly relative to the housing element results in axial displacement of the guiding element relative to the housing element up to penetration of the septum by the needle; and subsequently the plunger rod assembly being disengaged from the guiding element due to further axial displacement of the plunger rod assembly relative to the guiding element, which does not result in further axial displacement of the guiding element relative to the housing element.

Further preferably, the plunger rod assembly, comprising a plunger outer portion arranged along the longitudinal axis and a plunger inner portion arranged concentrically within the plunger outer portion and having at least one guiding groove arranged on the plunger outer portion; the plunger rod assembly is at least partially received into the housing element; a ring assembly mounted onto a portion of the housing element and having a radially inwardly extending protrusion engageable with a portion of the at least one guiding groove, whereas displacement of the radially inwardly extending protrusion along the guiding groove enables transition of the dual chamber injection system between the different operative orientations thereof.

Still further preferably, the different operative orientations comprising locked orientation, septum penetration, medicament reconstitution, priming and medicament injection. Yet further preferably, the ring assembly is being rotatable and engageable with the plunger outer portion, whereas axial displacement of the plunger rod assembly relative to the housing element is permitted upon rotation of the ring assembly relative to the housing element, thereby enabling transition of the dual chamber injection system between at least some of the different operative orientations thereof.

In accordance with an embodiment of the present invention, rotation of the ring assembly relative to the housing element results in alignment of corresponding indication features disposed on the housing element and the ring assembly, thereby enables indicating transition between the different operative orientations to the user.

Preferably, the axial displacement of the plunger assembly relative to the housing element results in displacement of at least one of the pistons contained within said medicament container, thereby enabling at least one of reconstitution of medicament, priming of the medicament container and injection of medicament. Further preferably, forward axial displacement of the plunger rod assembly relative to the housing element is prevented due to engagement of the ring assembly with the guiding groove in a locked operative orientation of the injection system.

Still further preferably, the ring assembly has a radially inwardly extending snap portion, which allows rotation of the ring assembly relative to the housing element in a first rotational direction and prevents rotation of the ring assembly in a second rotational direction, which is opposite to the first rotational direction. Yet further preferably, relative axial displacement between the plunger rod assembly and the guiding element is prevented in a locked operative orientation of the injection system, before initial axial displacement of the plunger assembly.

Preferably, the plunger rod assembly in a priming operative orientation is caused to be displaced axially forwardly due to thread-like engagement of the radially inwardly extending protrusion of the ring assembly with a helical priming portion of the guiding groove.

### BRIEF DESCRIPTION OF THE DRAWINGS

The present invention will be understood and appreciated more fully from the following detailed description, taken in conjunction with the drawings in which:

FIGS. 1A and 1B are respectively simplified pictorial view and exploded view of a dual chamber injection system constructed and operative in accordance with an embodiment of the present invention;

FIGS. 2A and 2B are respectively a simplified planar side view and a sectional view taken along lines B-B in FIG. 2A of an outer housing and a needle hub forming part of the dual chamber injection system of FIGS. 1A & 1B;

FIGS. 3A, 3B, 3C, 3D and 3E are respectively a simplified perspective view, two simplified plan side views and two simplified sectional views taken along lines D-D in FIG. 3C and E-E in FIG. 3D of a plunger rod outer portion forming part of the dual chamber injection system of FIGS. 1A & 1B;

FIGS. 4A, 4B, 4C, 4D and 4E are respectively a simplified perspective view, two simplified plan side views and two simplified sectional views taken along lines D-D and E-E in FIG. 4C of a plunger rod inner portion forming part of the dual chamber injection system of FIGS. 1A & 1B;

FIGS. 5A, 5B, 5C, 5D, 5E and 5F are respectively a simplified perspective view, two simplified plan side views and three simplified sectional views taken along lines D-D in FIG. 5B, E-E in FIG. 5D and F-F in FIG. 5E of a guiding and locking element forming part of the dual chamber injection system of FIGS. 1A & 1B in accordance with a first embodiment of the present invention;

FIGS. 6A, 6B and 6C are respectively a simplified perspective view, a simplified plan side view and a simplified sectional view taken along lines C-C in FIG. 6B of a guiding and locking element forming part of the dual chamber injection system of FIGS. 1A & 1B in accordance with a second embodiment of the present invention;

FIGS. 7A and 7B are simplified drawings of the dual chamber injection system of FIGS. 1A-5F in a locked operative orientation before attachment of a needle assembly, including respectively a simplified plan side view and a sectional view taken along lines B-B in FIG. 7A;

FIGS. 8A, 8B, 8C and 8D are simplified drawings of the dual chamber injection system of FIGS. 1A-5F in a locked operative orientation after attachment of a needle assembly, including respectively a simplified plan side view, plan top view and two sectional views taken along lines C-C and D-D in FIG. 8B;

FIGS. 8E and 8F are simplified drawings of the dual chamber injection system of FIGS. 1A-5F in the locked operative orientation after attachment of a needle assembly, including two different partially cut-out perspective views, FIG. 8E is shown without the outer housing of FIGS. 2A & 2B and FIG. 8F is shown without both the outer housing of FIGS. 2A & 2B and without the plunger rod outer portion of FIGS. 3A-3E;

FIGS. 9A, 9B, 9C and 9D are simplified drawings of the dual chamber injection system of FIGS. 1A-5F in an unlocked operative orientation, including respectively a simplified plan side view, plan top view and two sectional views taken along lines C-C and D-D in FIG. 9B;

FIGS. 9E and 9F are simplified drawings of the dual chamber injection system of FIGS. 1A-5F in the unlocked operative orientation, including two different partially cut-out perspective views, FIG. 9E is shown without the outer housing of FIGS. 2A & 2B and FIG. 9F is shown without both the outer housing of FIGS. 2A & 2B and without the plunger rod outer portion of FIGS. 3A-3E;

FIGS. 10A, 10B, 10C, 10D and 10E are simplified drawings of the dual chamber injection system of FIGS. 1A-5F in an end of reconstitution and prior to priming operative orientation, including respectively a simplified plan side view, plan top view and three sectional views taken along lines C-C, D-D and E-E in FIG. 10B;

FIGS. 10F and 10G are simplified drawings of the dual chamber injection system of FIGS. 1A-5F in the end of reconstitution and prior to priming operative orientation, including two different partially cut-out perspective views, FIG. 10F is shown without the outer housing of FIGS. 2A & 2B and FIG. 10G is shown without both the outer housing of FIGS. 2A & 2B and without the plunger rod outer portion of FIGS. 3A-3E;

FIGS. 11A, 11B, 11C, 11D and 11E are simplified drawings of the dual chamber injection system of FIGS. 1A-5F in an end of priming and prior to injection operative orientation, including respectively a simplified plan side view, plan top view and three sectional views taken along lines C-C, D-D and E-E in FIG. 11B;

FIGS. 11F and 11G are simplified drawings of the dual chamber injection system of FIGS. 1A-5F in the end of priming and prior to injection operative orientation, including two different partially cut-out perspective views, FIG. 11F is shown without the outer housing of FIGS. 2A & 2B and FIG. 11G is shown without both the outer housing of FIGS. 2A & 2B and without the plunger rod outer portion of FIGS. 3A-3E;

FIGS. 12A, 12B, 12C, 12D and 12E are simplified drawings of the dual chamber injection system of FIGS. 1A-5F in an end of injection locked operative orientation, including respectively a simplified plan side view, plan top view and three sectional views taken along lines C-C, D-D and E-E in FIG. 12B;

FIG. 12F is a simplified drawing of the dual chamber injection system of FIGS. 1A-5F in the end of injection locked operative orientation, including a partially cut-out perspective view, shown without both the outer housing of FIGS. 2A & 2B and without the plunger rod outer portion of FIGS. 3A-3E.

FIGS. 13A-13C are simplified drawings of three different embodiments of the assembled dual chamber injection systems;

FIGS. 14A and 14B are respectively simplified pictorial view and exploded view of a dual chamber injection system constructed and operative in accordance with another embodiment of the present invention;

FIGS. 15A-15D are respectively a simplified pictorial view, two different planar side views and a sectional view taken along lines D-D in FIG. 15B of a protective cap forming part of the dual chamber injection system of FIGS. 14A & 14B;

FIGS. 16A-16D are respectively two different simplified pictorial views, planar side view and a sectional view taken along lines D-D in FIG. 16C of a needle hub forming part of the dual chamber injection system of FIGS. 14A & 14B;

FIGS. 17A, 17B, 17C, 17D, 17E, 17F, 17G, 17H, 17I and 17J are respectively a simplified pictorial view, planar side view, sectional view taken along lines C-C in FIG. 17B, a different planar side view, three sectional views taken along lines E-E, F-F and G-G in FIG. 17D, a different pictorial view, a planar top view and sectional view taken along lines J-J in FIG. 17I of an outer housing forming part of the dual chamber injection system of FIGS. 14A & 14B;

FIGS. 18A, 18B, 18C, 18D, 18E, 18F. 18G, 18H, 18I, 18J and 18K are respectively a simplified pictorial view, planar side view, sectional view taken along lines C-C in FIG. 18B, a different planar side view, four sectional views taken along lines E-E, F-F, G-G and H-H in FIG. 18D, another different planar side view, a planar top view and sectional view taken along lines K-K in FIG. 18J of a syringe holder forming part of the dual chamber injection system of FIGS. 14A & 14B;

FIGS. 19A, 19B, 19C, 19D, 19E and 19F are respectively two different simplified pictorial views, a top planar view, a side planar view, and two sectional views taken along lines E-E and F-F in FIG. 19D of a ring portion forming part of the dual chamber injection system of FIGS. 14A & 14B;

FIGS. 20A-20D are respectively a simplified pictorial view, a side planar view, a top planar view and a sectional view taken along lines D-D in FIG. 20C of a collar forming part of the dual chamber injection system of FIGS. 14A & 14B;

FIGS. 21A-21D are respectively a simplified pictorial view, a planar side view and two simplified sectional views taken along lines C-C and D-D in FIG. 21B of a plunger rod outer portion forming part of the dual chamber injection system of FIGS. 14A & 14B;

FIGS. 22A-22D are respectively a simplified pictorial view, a planar side view and two simplified sectional views taken along lines C-C and D-D in FIG. 22B of a plunger rod inner portion forming part of the dual chamber injection system of FIGS. 14A & 14B;

FIGS. 23A-23C are simplified drawings of the dual chamber injection system of FIGS. 14A-22D in a locked operative orientation, including respectively two simplified different plan side views and a sectional view taken along lines C-C in FIG. 23B;

FIGS. 23D-23G are simplified drawings of the dual chamber injection system of FIGS. 14A-22D in a locked operative orientation, where only the outer housing, the plunger rod outer portion and one ring portion is shown to clearly describe the interactions therebetween, including respectively a plan side view and three different sectional views taken along lines E-E, G-G and F-F in FIG. 23D;

FIGS. 23H and 23I are simplified drawings of the dual chamber injection system of FIGS. 14A-22D in a locked operative orientation, including respectively a simplified plan side view and a sectional view taken along lines I-I in FIG. 23H;

FIGS. 23J-23M are simplified drawings of the dual chamber injection system of FIGS. 14A-22D in a locked operative orientation, including respectively a simplified plan side view and three sectional views taken along lines K-K, L-L and M-M in FIG. 23J, showing some of the components of the dual chamber injection system for clarity;

FIGS. 24A-24E are simplified drawings of the dual chamber injection system of FIGS. 14A-22D in an unlocked operative orientation, including respectively two simplified different plan side views and three sectional views taken respectively along lines C-C in FIG. 24B and lines D-D and E-E in FIG. 24A;

FIGS. 25A-25C are simplified drawings of the dual chamber injection system of FIGS. 14A-22D in a septum penetration operative orientation, including respectively two simplified different plan side views and a sectional view taken along lines C-C in FIG. 25B;

FIGS. 25D and 25E are simplified drawings of the dual chamber injection system of FIGS. 14A-22D in a septum penetration operative orientation, including respectively a simplified plan side view and a sectional view taken along lines E-E in FIG. 25D;

FIGS. 26A-26C are simplified drawings of the dual chamber injection system of FIGS. 14A-22D in a reconstitution operative orientation, including respectively two simplified different plan side views and a sectional view taken along lines C-C in FIG. 26B;

FIGS. 26D and 26E are simplified drawings of the dual chamber injection system of FIGS. 14A-22D in a reconstitution operative orientation, including respectively a simplified plan side view and a sectional view taken along lines E-E in FIG. 26D;

FIGS. 26F-26I are simplified drawings of the dual chamber injection system of FIGS. 14A-22D in a reconstitution operative orientation, where only the outer housing, the plunger rod outer portion and one ring portion is shown to clearly describe the interactions therebetween, including respectively a plan side view and three different sectional views taken along lines G-G, H-H and I-I in FIG. 26F;

FIGS. 27A-27C are simplified drawings of the dual chamber injection system of FIGS. 14A-22D in a priming operative orientation, including respectively two simplified different plan side views and a sectional view taken along lines C-C in FIG. 27B;

FIGS. 27D-27G are simplified drawings of the dual chamber injection system of FIGS. 14A-22D in a priming operative orientation, where only the outer housing, the plunger rod outer portion and one ring portion is shown to clearly describe the interactions therebetween, including respectively a plan side view and three different sectional views taken along lines E-E, F-F and G-G in FIG. 27D;

FIGS. 28A and 28B are simplified drawings of the dual chamber injection system of FIGS. 14A-22D in a cap removal operative orientation shown before removal of the protective cap, including respectively a simplified plan side view and a cut-out perspective view in FIG. 28B;

FIGS. 29A and 29B are simplified drawings of the dual chamber injection system of FIGS. 14A-22D in a cap removal operative orientation shown after removal of the protective cap, including respectively a simplified plan side view and a cut-out perspective view in FIG. 29B;

FIGS. 30A-30C are simplified drawings of the dual chamber injection system of FIGS. 14A-22D in an end of injection operative orientation, including respectively two simplified different plan side views and a sectional view taken along lines C-C in FIG. 30B;

FIGS. 30D-30G are simplified drawings of the dual chamber injection system of FIGS. 14A-22D in an end of injection operative orientation, where only the outer housing, the plunger rod outer portion and one ring portion is shown to clearly describe the interactions therebetween, including respectively a plan side view and three different sectional views taken along lines E-E, F-F and G-G in FIG. 30D.

### DESCRIPTION OF EMBODIMENTS

The principles, uses and implementations of the teachings herein may be better understood with reference to the accompanying description and figures. Upon perusal of the description and figures present herein, one skilled in the art is able to implement the invention without undue effort or experimentation.

Before explaining at least one embodiment of the invention in detail, it is to be understood that the invention is not limited in its applications to the details of construction and the arrangement of the components and/or methods set forth in the following description and/or illustrated in the drawings and/or the Examples. The invention can be implemented with other embodiments and can be practiced or carried out in various ways. It is also understood that the phraseology and terminology employed herein is for descriptive purpose and should not be regarded as limiting.

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

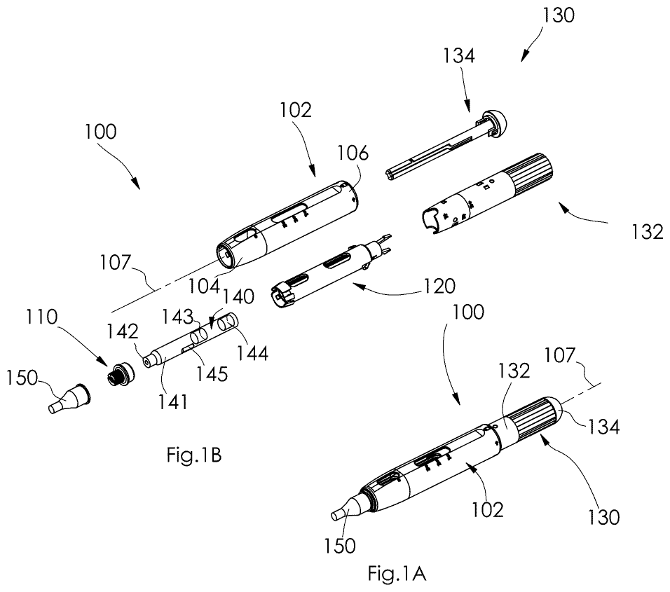

Reference is now made to FIGS. 1A and 1B, which are respectively simplified pictorial view and exploded view of a dual chamber injection system constructed and operative in accordance with an embodiment of the present invention.

As seen in FIGS. 1A & 1B, a dual chamber injection system 100 preferably includes an outer housing 102 having a forward end 104 and a rearward end 106 and is arranged along a longitudinal axis 107. A needle hub 110 is configured to be fixedly coupled to the forward end 104 of the outer housing 102 or integrally made therewith. A guiding and locking element 120 is configured to be at least partially received into the outer housing 102 and fixedly attached thereto or integrally made therewith.

A plunger rod assembly 130 is configured to be partially inserted into the rearward end 106 of the outer housing 102 and adapted to be moveable relative to the outer housing 102. The plunger rod assembly 130 preferably includes an outer plunger portion 132 and an inner plunger portion 134, which are fixedly attached to each other. Alternatively, the outer plunger portion 132 and the inner plunger portion 134 may be integrally made as a single component.

In accordance with an embodiment of the present invention, the inner plunger portion 134 is at least partially received into the outer plunger portion 132 and arranged coaxially therewith along the longitudinal axis 107.

It is a particular feature of an embodiment of the present invention that the outer housing 102 is operatively associated with the plunger assembly 130, such that certain axial and/or rotational orientations of the plunger assembly 130 relative to the outer housing 102. result in alignment of corresponding indication features disposed on the outer housing 102 and the plunger assembly 130, thereby enable indicating to the user what is the operative state that the dual chamber injection system 100 is situated in.

It is noted that a dual chamber medicament cartridge 140 is adapted to be inserted into the outer housing 102 and the forward end thereof is adapted to be supported against the needle hub 110.

It is appreciated that the dual chamber medicament cartridge 140 can be any type of conventional cartridge, such as a cartridge commercially available from Schott Pharmaceutical Systems, Mainz, Germany or Vetter Pharma International USA Inc., IL, USA or Nuova Ompi S.r.l., Padua, Italy or may be any other suitable syringe or cartridge.

The dual chamber medicament cartridge 140 preferably includes a cartridge barrel 141 and a septum 142 at a forward end thereof. Preferably, two pistons, namely a forward piston 143 and a rearward piston 144 are contained within the cartridge barrel 141 and are adapted for slidable axial displacement relative thereto. It is appreciated that a drug preparation is preferably confined between the forward piston 143 and the septum 142 and a solvent is preferably confined between the forward piston 143 and the rearward piston 144 and upon appropriate longitudinal displacement of the pistons, the two substances are configured for reconstitution and subsequent ejection, as described in detail hereinbelow.

The cartridge barrel 141 has a generally cylindrical shape and extends along the longitudinal axis 107. At least one bypass protrusion 145 is disposed generally at an intermediate location of the cartridge barrel 141. The bypass protrusion 145 generally extends radially outwardly from an outer surface of the cartridge barrel 141 to facilitate fluid passage between the two chambers formed within the cartridge barrel 141, one between the two pistons 143 and 144 and another one between the forward piston 143 and the septum 142.

A needle cover 150 is adapted to be mounted onto the needle hub 110.

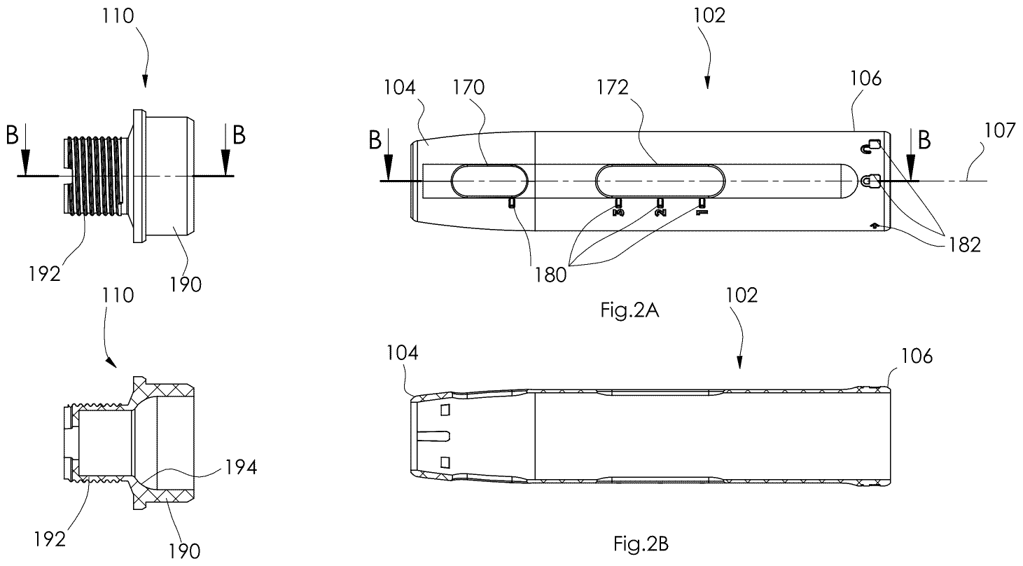

Reference is now made to FIGS. 2A and 2B, which are respectively a simplified planar side view and a sectional view taken along lines B-B in FIG. 2A of the outer housing 102 and the needle hub 110 forming part of the dual chamber injection system 100 of FIGS. 1A & 1B.

The outer housing 102 and the needle hub 110 may be fixedly attached to each other and may alternatively be integrally formed as a single component.

In accordance with an embodiment of the present invention, the outer housing 102 is an integrally formed element, preferably injection molded of plastic and is arranged along longitudinal axis of symmetry 107.

The outer housing 102 is a generally longitudinal hollow cylindrical element having forward end 104 and rearward end 106. Preferably, a forward window 170 is formed adjacent the forward end 104 and a rearward window 172 is formed at a generally intermediate location along the longitudinal extent of the outer housing 102.

A plurality of demarcations 180 are preferably provided on the outer housing 102 adjacent to each of the forward and rearward windows 170 and 172, as shown particularly in FIG. 2A. Additionally, a plurality of demarcations 182 are preferably provided adjacent to the rearward end 106 of the outer housing, as also particularly shown in FIG. 2A.

The needle hub 110 is an integrally formed element, preferably injection molded of plastic and is arranged along longitudinal axis of symmetry 107.

The needle hub 110 preferably has a generally cylindrical hub portion 190 adapted to be fixedly attached to the outer housing 102 and an outwardly threaded portion 192 forwardly extending therefrom.

A generally curved inner surface 194 disposed between the hub portion 190 and the outwardly threaded portion 192 is adapted for forwardly supporting the medicament cartridge 140.

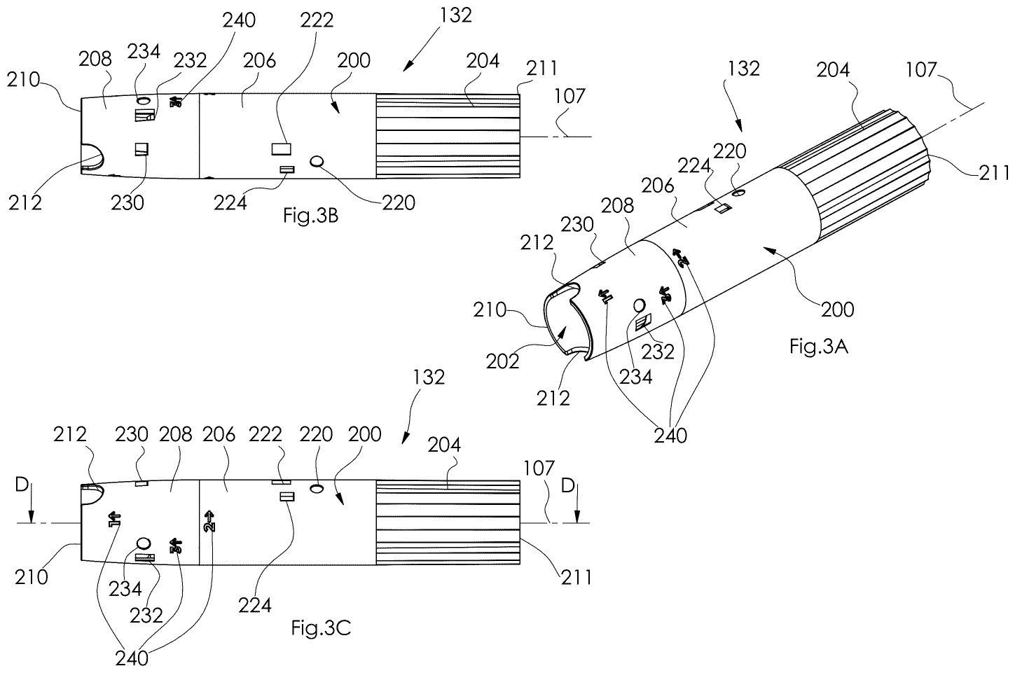

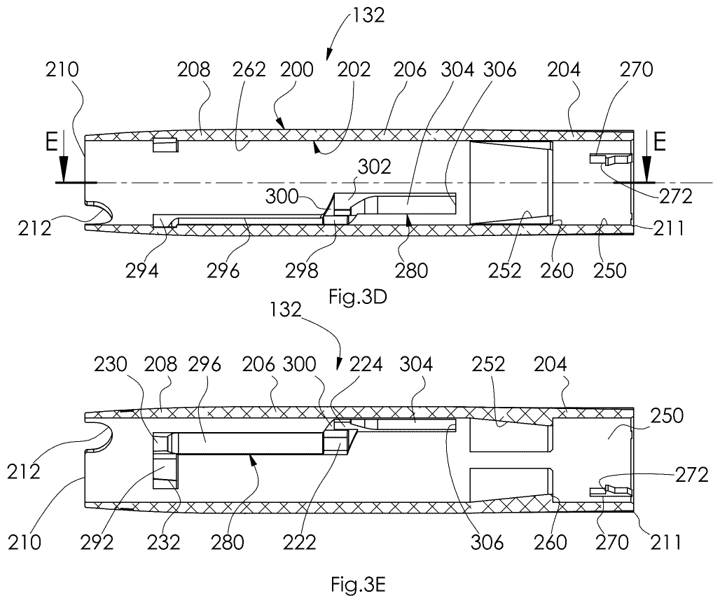

Reference is now made to FIGS. 3A, 3B, 3C, 3D and 3E, which are respectively a simplified perspective view, two simplified plan side views and two simplified sectional views taken along lines D-D in FIG. 3C and E-E in FIG. 3D of the plunger rod outer portion 132 forming part of the dual chamber injection system 100 of FIGS. 1A & 1B.

The plunger rod outer portion 132 is an integrally formed element, preferably injection molded of plastic and is arranged along longitudinal axis of symmetry 107.

It is seen in FIGS. 3A-3C that the plunger rod outer portion 132 defines an outer surface 200 and an inner surface 202. The plunger rod outer portion 132 generally has a rearward portion 204 preferably having gripping protrusions formed thereon, a middle portion 206 having a generally circular cross-section and a forward portion 208, being generally forwardly tapered and terminating at a forwardmost edge 210. The rearward portion 204 defines a rearward most edge 211.

Typically, two arcuate openings 212 are formed in the forward portion 208 and being mutually diametrically opposed to each other and extend generally rearwardly from the forwardmost edge 210.

It is seen in FIGS. 3A-3C that a demarcation 220 is provided on the middle portion 206 of the plunger rod outer portion 132.

It is noted that all demarcations provided on the plunger rod outer portion 132 may be printed thereon or may alternatively be in a form of a protrusion or a recess.

It is further seen in FIGS. 3A-3C that typically two openings 222 and 224 are formed on the middle portion 206 of the plunger rod outer portion 132. It is noted that the two openings 222 and 224 are disposed in different axial and rotational orientations relative to each other. It is seen that opening 224 is preferably forwardly spaced from demarcation 220 and opening 222 is preferably slightly forwardly spaced from opening 224 and is radially spaced therefrom.

It is additionally seen in FIGS. 3A-3C that typically two openings 230 and 232 are formed on the forward portion 208 of the plunger rod outer portion 132. It is noted that the two openings 230 and 232 are axially aligned and radially spaced from each other.

A demarcation 234 is also provided on the forward portion 208 of the plunger rod outer portion 132 and is disposed adjacent the opening 232, generally axially aligned therewith and radially spaced therefrom.

A plurality of demarcations 240 are preferably provided on both the middle portion 206 and the forward portion 208 of the plunger rod outer portion 132.

Turning now specifically to FIGS. 3D & 3E, it is seen that an inner volume of the plunger rod outer portion 132 preferably includes a rearward portion 250, having a preferably circular cross-section and extending forwardly from the rearward most edge 211, a conical portion 252 extending forwardly from the rearward portion 250 and forming a rearwardly facing generally circumferential locking shoulder 260 therebetween. A relatively long forward portion 262 preferably having a circular cross-section extends forwardly from the conical portion 252 to the forwardmost edge 210.

A snap protrusion 270 is generally formed adjacent the rearward most edge 211, defining a generally forwardly facing shoulder 272 and adapted for fixed coupling with the plunger rod inner portion 134.

It is a particular feature of an embodiment of the present invention that typically, two diametrically opposed guiding tracks 280 are formed on the inner surface 202 of the plunger rod outer portion 132 and disposed along at least a portion of the longitudinal extent of the forward portion 262.

The guiding track 280 includes an unlocking groove 292, which extends radially from opening 232 preferably in parallel to the plane of the forwardmost edge 210, and terminates at opening 230. A reconstitution groove 296 extends axially rearwardly from opening 230 up to opening 222. The reconstitution groove 296 generally extends in parallel to the longitudinal axis 107. At least partially helical priming groove 300 extends both radially and axially rearwardly from opening 222 to opening 224. An injection groove 304 extends axially rearwardly from opening 224 to a forwardly facing shoulder 306, located generally adjacent to the conical portion 252.

It is a further particular feature of an embodiment of the present invention that the guiding track 280 is a single continuous guiding track having portions thereof that are disposed in different axial and angular orientations along the inner surface 202 of the plunger rod outer portion 132. It is appreciated that alternatively, the guiding track 280 may be formed of several separate grooves and openings, which are spaced from each other. It is further appreciated that alternatively, the guiding track 280 may be eliminated and the outwardly extending protrusion 424 of the indicating arm 420 may be displaceable between the openings 232, 230 222 and 224 along the inner surface 202 of the plunger rod outer portion 132.

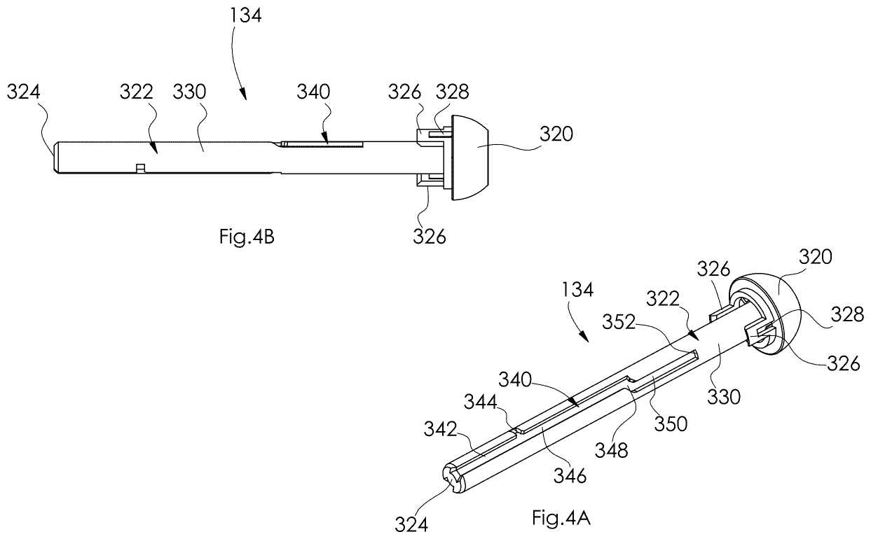

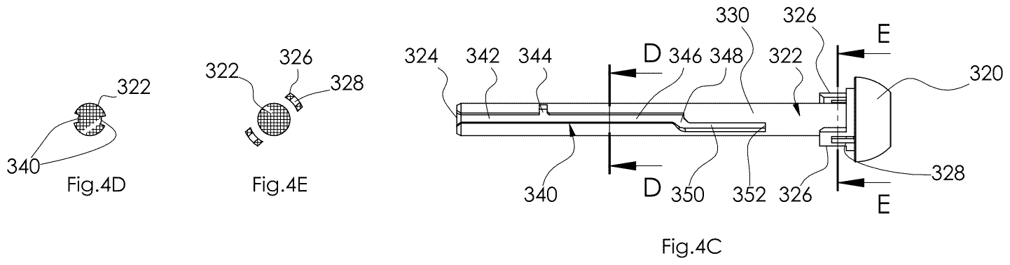

Reference is now made to FIGS. 4A, 4B, 4C, 4D and 4E, which are respectively a simplified perspective view, two simplified plan side views and two simplified sectional views taken along lines D-D and E-E in FIG. 4C of the plunger rod inner portion 134 forming part of the dual chamber injection system 100 of FIGS. 1A & 1B.

The plunger rod inner portion 134 is an integrally formed element, preferably injection molded of plastic and is arranged along longitudinal axis of symmetry 107.

It is seen in FIGS. 4A-4E that the plunger rod inner portion preferably includes a hub 320 and a longitudinal shaft 322 extending axially forwardly therefrom to a forwardly facing surface 324. Typically, two diametrically opposed couplers 326 extend forwardly from the hub 320 and define a window 328 therewithin.

The longitudinal shaft 322 is preferably cylindrical and defines an outer surface 330.

It is a particular feature of an embodiment of the present invention that typically, two diametrically opposed guiding grooves 340 are formed on the outer surface 330 of the longitudinal shaft 322 and extends typically from the forwardly facing surface 324 towards the hub 320.

It is specifically seen that the guiding groove 340 preferably includes an assembly groove portion 342 extending axially rearwardly from the forwardly facing surface 324 to a locking groove portion 344, which extends slightly radially from the assembly groove portion 342. It is noted that the locking groove portion 344 preferably extends transversely relative to the assembly groove portion 342.

It is further seen in FIGS. 4A-4E that a reconstitution groove portion 346 extends axially rearwardly from the locking groove portion 344 and is preferably radially aligned with the assembly groove portion 342. It is noted that the reconstitution groove portion 346 preferably extends transversely relative to the locking groove portion 344. The reconstitution groove portion 346 extends axially rearwardly up to a helical priming groove portion 348 which extends both radially and axially rearwardly from the reconstitution groove portion 346.

An injection groove portion 350 extends axially rearwardly from the priming groove portion 348 up to a forwardly facing surface 352.

It is a further particular feature of an embodiment of the present invention that the guiding groove 340 is a single continuous guiding groove having portions thereof that are disposed in different axial and angular orientations along the outer surface 330 of the longitudinal shaft 322 of the plunger rod inner portion 134, such as the assembly groove portion 342, the locking groove portion 344, the reconstitution groove portion 346, the helical priming groove portion 348 and the injection groove portion 350.

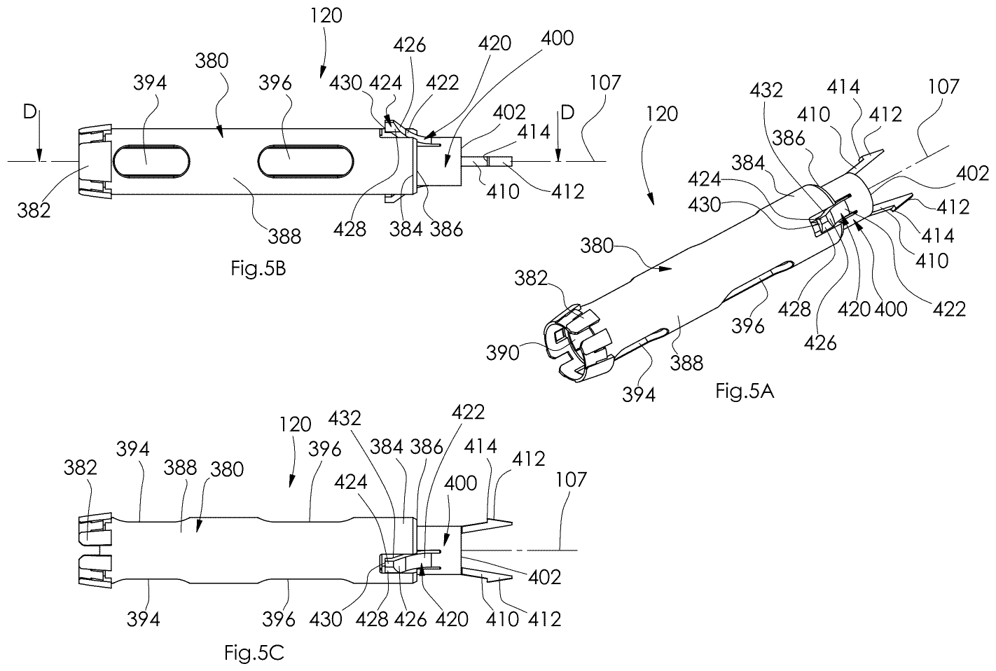

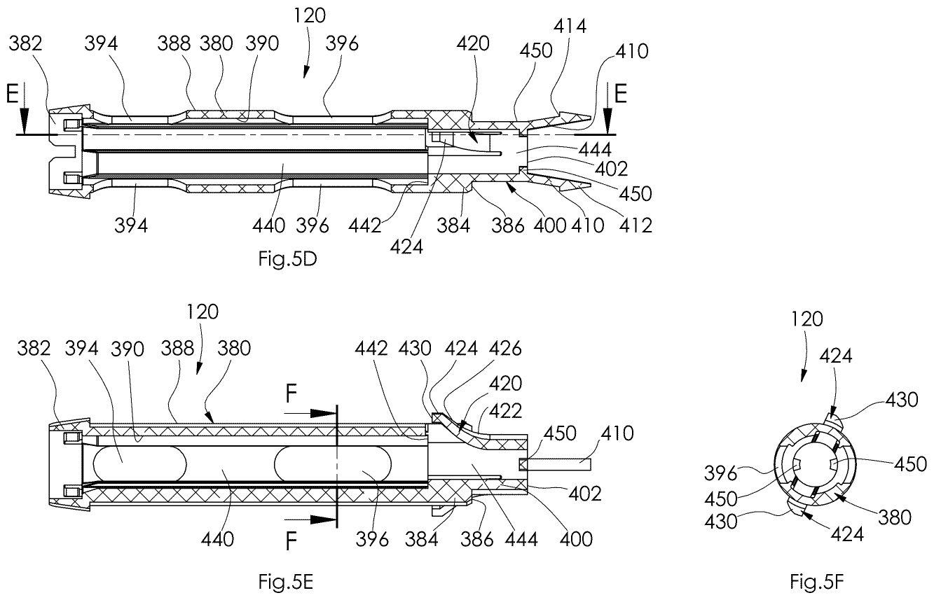

Reference is now made to FIGS. 5A, 5B, 5C, 5D, 5E and 5F, which are respectively a simplified perspective view, two simplified plan side views and three simplified sectional views taken along lines D-D in FIG. 5B, E-E in FIG. 5D and F-F in FIG. 5E of the guiding and locking element 120 forming part of the dual chamber injection system 100 of FIGS. 1A & 1B in accordance with a first embodiment of the present invention.

The guiding and locking element 120 is an integrally formed element, preferably injection molded of plastic and is arranged along longitudinal axis of symmetry 107. It is noted that the guiding and locking element 120 acts as a syringe holder in the dual chamber injection system 100.

It is seen in FIGS. 5A-5F that the guiding and locking element 120 preferably includes a generally cylindrical longitudinal sleeve portion 380 having a forward end including a plurality of snap protrusions 382, which are disposed circumferentially along the forward end of the guiding and locking element 120 and generally protrude radially outwardly therefrom. The sleeve portion 380 extends axially to a rearward end 384, defining a rearwardly facing shoulder 386. The snap protrusions 382 are configured for fixed attachment with the outer housing 102.

The sleeve portion 380 defines an outer surface 388 and an inner surface 390.

It is seen that typically, two pairs of windows, diametrically opposed to each other, are formed in the sleeve portion 380. A forward window 394 and a rearward window 396.

It is seen in FIGS. 5A-5F that a generally cylindrical guiding portion 400 extends axially rearwardly from the sleeve portion 380. The guiding portion 400 extends rearwardly from the rearwardly facing shoulder 386 to a rearward most edge 402. Typically, two locking arms 410 extend rearwardly from the rearward most edge 402 and slightly radially outwardly with respect thereto. The locking arms 410 each include a lateral finger protrusion 412 defining a forwardly facing locking surface 414.

It is a particular feature of an embodiment of the present invention that typically, two diametrically opposed indicating arms 420 extend axially forwardly and radially outwardly from the guiding portion 400 and at least partially extend through a rearward end of the sleeve portion 380. The indicating arms 420 are connected to the guiding portion 400 and extend forwardly therefrom and at angle with respect thereto. The indicating arms 420 preferably include a generally curved portion 422 and a radially outwardly extending protrusion 424. The protrusion 424 preferably includes a rearwardly facing tapered surface 426, joining the curved portion 422 with the radially outwardly extending protrusion 424. The protrusion 424 also preferably includes a generally laterally facing tapered surface 428, a forwardly facing surface 430, extending generally transversely with respect to the longitudinal axis 107 and a generally laterally facing surface 432, facing an opposite direction as compared with laterally facing tapered surface 428 and extending generally transversely with respect to the longitudinal axis 107.

It is a further particular feature of an embodiment of the present invention that the indicating arms 420 are configured to be radially inwardly deflectable upon application of pressure thereon.

Turning now specifically to FIGS. 5D-5F, the inner volume of the guiding and locking element 120 includes a longitudinal portion 440 extending along the sleeve portion 380 and terminating at a forwardly facing shoulder 442, adapted for supporting the medicament cartridge 140. The inner volume of the guiding and locking element 120 also includes a rearward portion 444 extending rearwardly of the longitudinal portion 440 along the guiding portion 400 and terminating at the rearward most edge 402.

It is a particular feature of an embodiment of the present invention that typically, two diametrically opposed guiding teeth 450 extend radially inwardly from the rearward portion 444 of the guiding and locking element 120. The guiding teeth 450 are preferably disposed adjacent the rearward most edge 402 and extend radially inwardly from the inner surface 390 of the guiding and locking element 120.

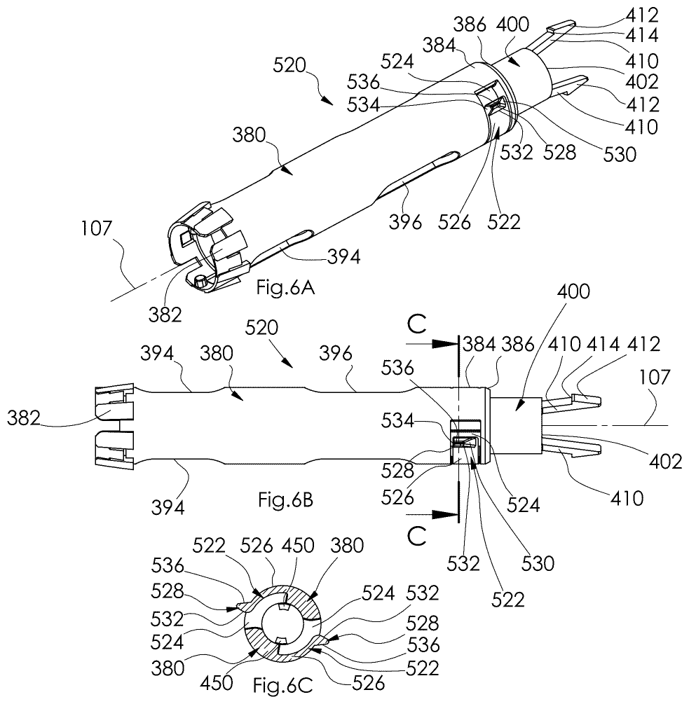

Reference is now made to FIGS. 6A, 6B and 6C, which are respectively a simplified perspective view, a simplified plan side view and a simplified sectional view taken along lines C-C in FIG. 6B of a guiding and locking element forming part of the dual chamber injection system 100 of FIGS. 1A & 1B in accordance with a second embodiment of the present invention.

It is appreciated that a guiding and locking element 520 in accordance with an alternative embodiment of the present invention, is substantially identical to guiding and locking element 120, as illustrated in FIGS. 5A-5F, besides the indicating arms 420, which are designed differently, such as indicating arms 522 in this embodiment of the present invention, illustrated in FIGS. 6A-6C. The remaining elements of the guiding and locking element 522 which are identical to elements of the guiding and locking element 120 are designated by the same reference numerals.

It is a particular feature of an embodiment of the present invention that typically, two diametrically opposed indicating arms 522 are disposed within openings 524 formed in the sleeve portion 380 of the guiding and locking element 520, adjacent to the rearward end 384 thereof. The indicating arms 522 extend radially circumferentially within the sleeve portion 380. The two indicating arms 522 are preferably radially spaced from each other and arranged along the circumference of the sleeve portion 380. The indicating arms 522 preferably include a generally curved portion 526 and a radially outwardly extending protrusion 528.

The protrusion 528 preferably includes a rearwardly facing tapered surface 530. The protrusion 528 also preferably includes a generally laterally facing tapered surface 532, joining the curved portion 526 with the radially outwardly extending protrusion 528. The protrusion 528 also preferably includes a forwardly facing surface 534, extending generally transversely with respect to the longitudinal axis 107 and a generally laterally facing surface 536, facing an opposite direction as compared with laterally facing tapered surface 532 and extending generally transversely with respect to the longitudinal axis 107.

It is a further particular feature of an embodiment of the present invention that the indicating arms 522 are configured to be radially inwardly deflectable upon application of pressure thereon.

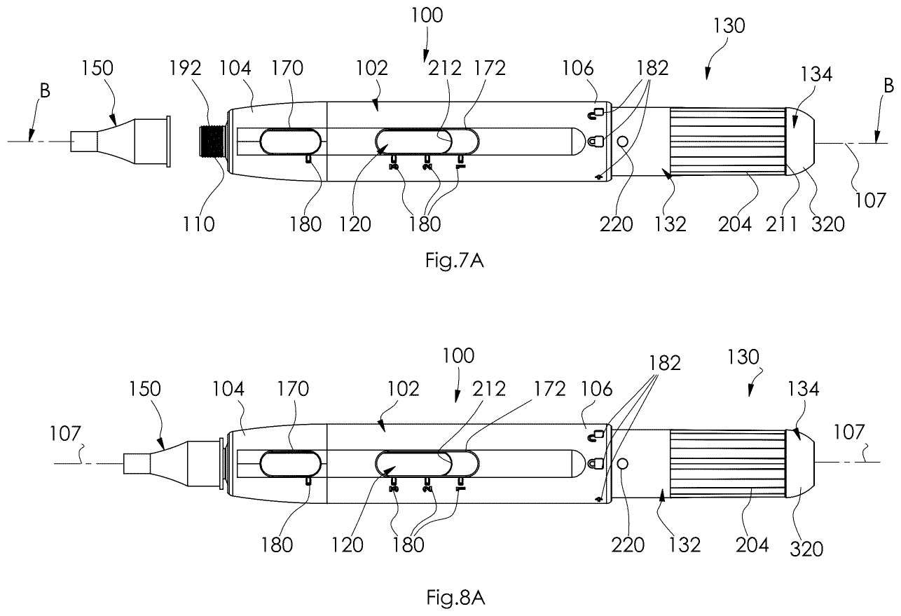

Reference is now made to FIGS. 7A and 7B, which are simplified drawings of the dual chamber injection system 100 of FIGS. 1A-5F in a locked operative orientation before attachment of the needle assembly 150, including respectively a simplified plan side view and a sectional view taken along lines B-B in FIG. 7A.

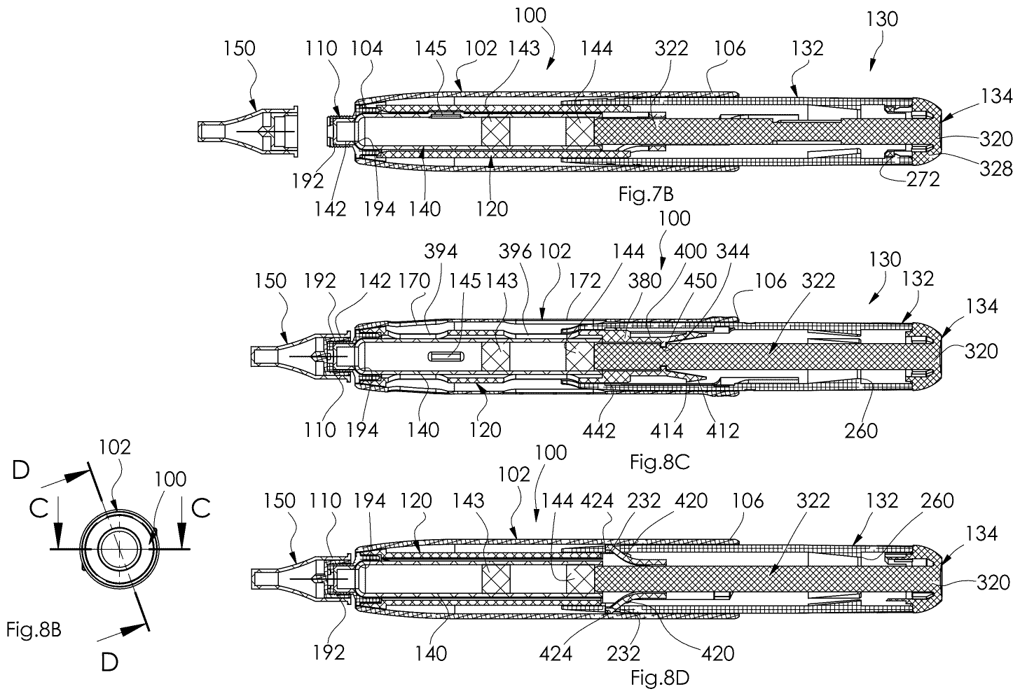

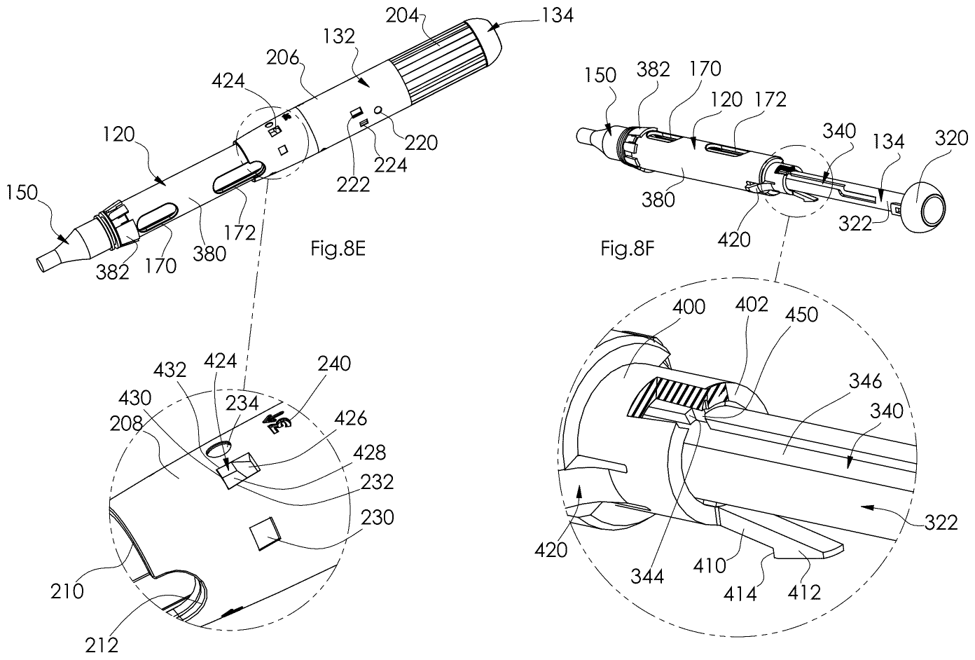

Reference is additionally made to FIGS. 8A, 8B, 8C and 8D, which are simplified drawings of the dual chamber injection system 100 of FIGS. 1A-5F in a locked operative orientation after attachment of the needle assembly 150, including respectively a simplified plan side view, plan top view and two sectional views taken along lines C-C and D-D in FIG. 8B and to FIGS. 8E and 8F, which are simplified drawings of the dual chamber injection system 100 of FIGS. 1A-5F in the locked operative orientation after attachment of the needle assembly 150, including two different partially cut-out perspective views, FIG. 8E is shown without the outer housing 102 of FIGS. 2A & 2B and FIG. 8F is shown without both the outer housing 102 of FIGS. 2A & 2B and without the plunger rod outer portion 132 of FIGS. 3A-3E.

The dual chamber injection system 100 is seen in FIGS. 7A & 7B before the attachment of the needle assembly 150 to the needle hub 110. The dual chamber injection system 100 is seen in FIGS. 8A-8F following attachment of the needle assembly 150 to the needle hub 110, such that the needle penetrates the septum 142 of the medicament cartridge 140 and fluid communication is established between the needle and the inner volume of the medicament cartridge 140.

It is specifically seen in FIGS. 7B, 8C and 8D that plunger rod inner portion 134 is generally received into the plunger rod outer portion 132, whereas the hub 320 of the plunger rod inner portion 134 protrudes rearwardly from the plunger rod outer portion 132. It is noted that the plunger rod inner portion 134 is fixedly coupled to the plunger rod outer portion 132 by means of engagement between forwardly facing shoulder 272 of the plunger rod outer portion 132 with window 328 of the plunger rod inner portion 134. The plunger rod inner portion 134 is disposed concentrically with the plunger rod outer portion 132. It is appreciated that alternatively the plunger rod outer portion 132 and the plunger rod inner portion 134 may be formed integrally as a single element.

It is further seen in FIGS. 7B, 8C and 8D that the plunger rod assembly 130 is partially inserted into the outer housing 102 and protrudes rearwardly therefrom.

It is further seen that the needle hub 110 is fixedly coupled to the forward end 104 of the outer housing 102 and the guiding and locking element 120 is inserted into the outer housing 102 and is partially inserted between the plunger rod inner portion 134 and the plunger rod outer portion 132 and concentrically arranged therewith. The needle hub 110 is fixedly attached to both the outer housing 102 and to the guiding and locking element 120. It is seen that at least the rearward end of the sleeve portion 380 and the guiding portion 400 of the guiding and locking element 120 are disposed between the plunger rod outer portion 132 and the plunger rod inner portion 134 in this locked operative orientation of the dual chamber injection system 100.

It is seen that in this locked operative orientation of the dual chamber injection system 100, the guiding portion 400 of the guiding and locking element 120 is forwardly spaced from locking shoulder 260 of the plunger rod outer portion 132.

It is particularly seen n FIGS. 7B, 8C and 8D that the medicament cartridge 140 is received into the guiding and locking element 120 and is supported on both sides thereof between the curved inner surface 194 of the needle hub 110 and the forwardly facing shoulder 442 of the guiding and locking element 120.

It is seen that in this locked operative orientation the pistons 143 and 144 are rearwardly spaced relative to the bypass protrusion 145. The rearward piston 144 is rearwardly spaced from the forward piston 143 and the plunger rod inner portion 134 engages the rearward piston 144.

It is a particular feature of an embodiment of the present invention that the guiding teeth 450 of the guiding and locking element 120 are engaged with the corresponding guiding grooves 340 of the plunger rod inner portion 134. It is particularly seen in FIGS. 8C and 8F that the guiding teeth 450 are disposed in the locking groove portion 344 of guiding grooves 340 in this locked operative orientation.

It is appreciated that in this locked operative orientation of the dual chamber injection system 100, the plunger assembly 130 is prevented from axial displacement relative to the guiding and locking element 120 either forwardly or rearwardly due to engagement of the guiding teeth 450 with the locking groove portion 344, which extends laterally from the axial portions of the guiding groove 340, such as the assembly groove portion 342 and the reconstitution groove portion 346. Additionally, the engagement of the guiding teeth 450 with the locking groove portion 344 prevents rotation of the plunger assembly 130 relative to the guiding and locking element 120 in a counter-clockwise direction. It is noted that the rotation direction can be reversed according to another embodiment of the present invention.

It is also appreciated that the plunger assembly 130 is also restricted from rearward axial displacement relative to the guiding and locking element 120 and the outer housing 102 by means of engagement of the radially outwardly extending protrusion 424 with opening 232 in the outer plunger portion 132, specifically by means of engagement of forwardly facing surface 430 with a face of opening 232. Additionally, the engagement of radially outwardly extending protrusion 424 with opening 232 restricts rotational displacement of the plunger assembly 130 relative to the guiding and locking element 120 in a counter-clockwise direction, specifically by means of engagement of laterally facing surface 432 with a face of opening 232. It is noted that the rotation direction can be reversed according to another embodiment of the present invention.

It is noted that the guiding teeth 450 of the guiding and locking element 120 are guided along the assembly groove portion 342 during assembly of the dual chamber injection system 100 and are seated within the locking groove portion 344 in storage, in the locked operative orientation.

It is a particular feature of an embodiment of the present invention that the displacement of the plunger assembly 130 relative to the guiding and locking element 120 is guided by engagement of the guiding teeth 450 of the guiding and locking element 120 with the guiding groove 340 of the plunger rod inner portion 134.

It is a further particular feature of an embodiment of the present invention that a tactile and audial indication is provided to the user to indicate transitions between different operative orientations during the process of medicament reconstitution, priming and injection of the medicament from the medicament cartridge 140. This indication to the user is provided by engagement of the indicating arms 420 of the guiding and locking element 120 with the guiding tracks 280 of the plunger rod outer portion 132.

It is particularly seen in FIGS. 8D and 8E that the outwardly extending protrusions 424 of the indicating arms 420 are seated within openings 232 of the plunger rod outer portion 132. It is noted that the indicating arms 420 are relatively resilient and can be removed from openings 232 upon axial displacement or rotation of the plunger assembly 130, due to the tapered configuration of surfaces 426 and 428 of the indicating arms 420 respectively.

It is also seen in FIG. 8C that windows 170 and 172 of the guiding and locking element 120 are preferably respectively aligned with windows 394 and 396 of the outer housing 102.

It is a particular feature of an embodiment of the present invention, as seen both in FIG. 8E and in FIGS. 7A & 7B, that openings 212 of the plunger rod outer portion 132 are aligned with windows 172 and 396, thus providing a visual indication to the user about transitions between different operative orientations during the process of medicament reconstitution, priming and injection of the medicament from the medicament cartridge 140.

Specifically, in this locked operative orientation, it is seen in FIGS. 7A and 8A that openings 212 of the plunger rod outer portion 132 appear within windows 172 of the outer housing and are aligned with one of the demarcations 180 of the outer housing 102, preferably designated as “1”.

It is a further particular feature of an embodiment of the present invention that an additional visual indication is provided to the user by means of alignment of one of the demarcations 182 of the outer housing 102 with demarcation 220 of the plunger rod outer portion 132. In this locked operative orientation, the demarcation 220 is preferably aligned with a lock sign.

It is additionally noted that the user can inspect the medicament through window 170 of the outer housing 102, which is aligned with window 394 of the guiding and locking element 120, as well as through window 172 of the outer housing 102, which is aligned with window 396 of the guiding and locking element 120.

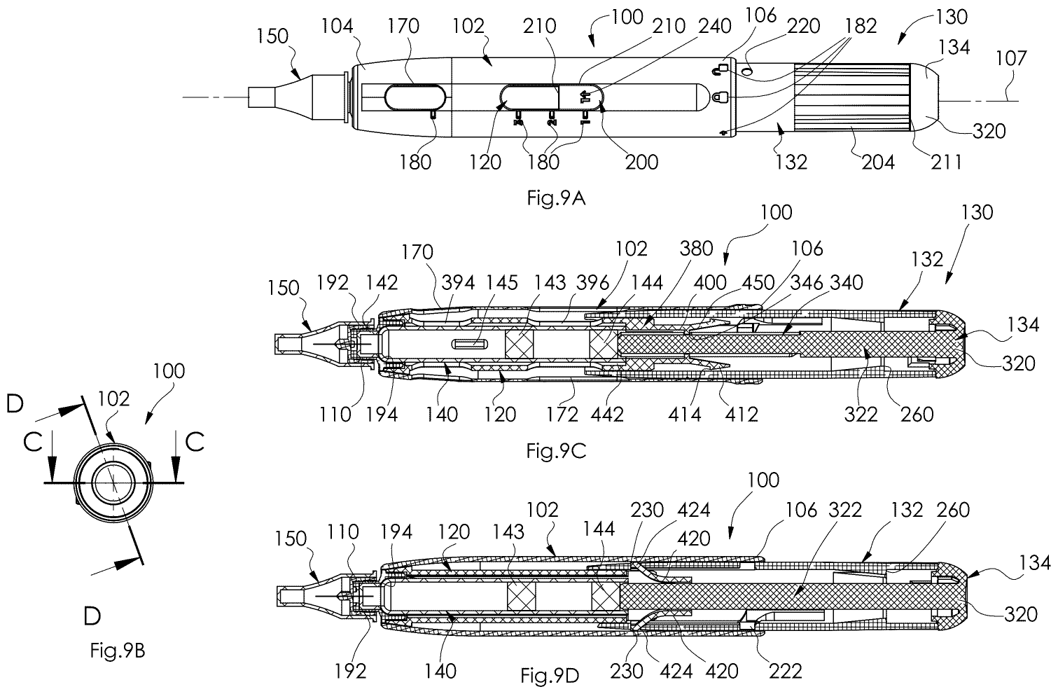

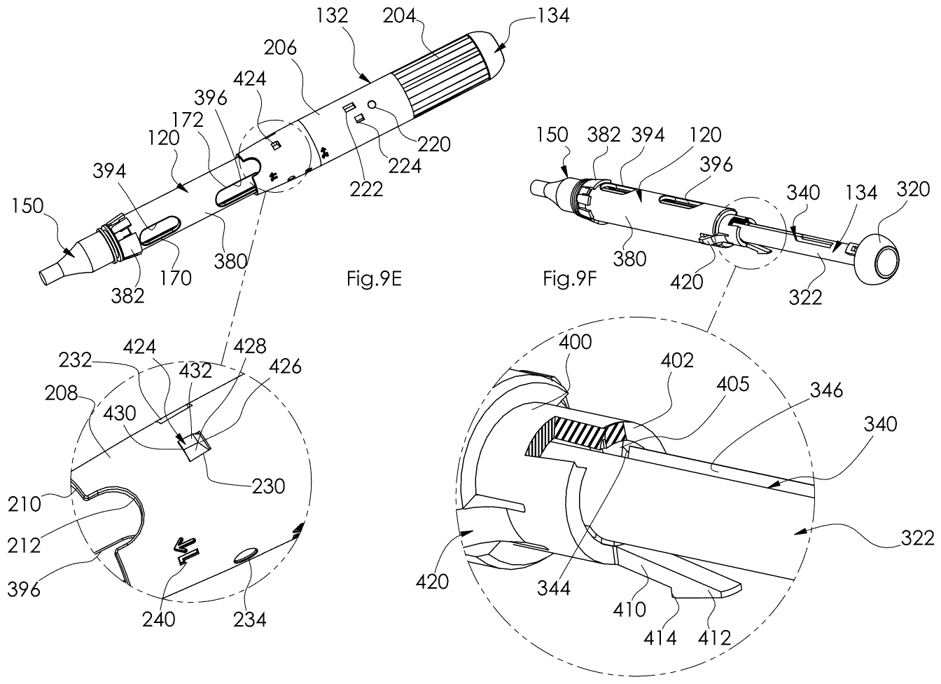

Reference is now made to FIGS. 9A, 9B, 9C and 9D, which are simplified drawings of the dual chamber injection system 100 of FIGS. 1A-5F in an unlocked operative orientation, including respectively a simplified plan side view, plan top view and two sectional views taken along lines C-C and D-D in FIG. 9B. Reference is additionally made to FIGS. 9E and 9F, which are simplified drawings of the dual chamber injection system 100 of FIGS. 1A-5F in the unlocked operative orientation, including two different partially cut-out perspective views, FIG. 9E is shown without the outer housing of FIGS. 2A & 2B and FIG. 9F is shown without both the outer housing 102 of FIGS. 2A & 2B and without the plunger rod outer portion 132 of FIGS. 3A-3E.

The dual chamber injection system 100 is seen in FIGS. 9A-9F in the unlocked operative orientation, before initiation of medicament reconstitution. It is seen particularly in FIGS. 9A-9F that the plunger rod assembly 130 is disposed in the same axial orientation relative to the outer housing 102 as shown in FIGS. 8A-8F. It is also particularly seen in FIGS. 9A-9F that the plunger rod assembly 130 is rotated about longitudinal axis 107, generally in a clockwise direction, relative to the outer housing 102, as compared to the locked orientation of the dual chamber injection system 100 shown in FIGS. 8A-8F.

It is seen that in this unlocked operative orientation of the dual chamber injection system 100, the guiding portion 400 of the guiding and locking element 120 remains forwardly spaced from locking shoulder 260 of the plunger rod outer portion 132.

It is seen that in this unlocked operative orientation that the pistons 143 and 144 remain rearwardly spaced relative to the bypass protrusion 145. The rearward piston 144 is rearwardly spaced from the forward piston 143 and the plunger rod inner portion 134 engages the rearward piston 144.

It is noted that most of the spatial relationships between the different components of the dual chamber injection system 100 remain the same as described and illustrated with respect to FIGS. 8A-8F, besides the following:

It is a particular feature of an embodiment of the present invention that during rotation of the plunger rod assembly 130 relative to the outer housing 102, the plunger rod assembly 130 also rotates relative to the guiding and locking element 120, due to fixed coupling between the guiding and locking element 120 with the outer housing 102. It is particularly seen in FIGS. 9C & 9F that upon rotation of the plunger rod assembly 130 relative to guiding and locking element 120, the guiding teeth 450 of the guiding and locking element 120 disengage the locking groove portion 344 of guiding grooves 340 of the plunger rod inner portion 134 in this unlocked operative orientation and the guiding teeth 450 are now disposed at the forward end of the reconstitution groove portion 346 of the guiding grooves 340.

It is appreciated that in this unlocked operative orientation of the dual chamber injection system 100, the plunger assembly 130 is permitted to be axially forwardly displaced relative to the guiding and locking element 120 due to engagement of the guiding teeth 450 with the reconstitution groove portion 346. Additionally, the engagement of the guiding teeth 450 with the reconstitution groove portion 346 prevents rotation of the plunger assembly 130 relative to the guiding and locking element 120 in both directions.

It is also appreciated that the plunger assembly 130 is also restricted from rearward axial displacement relative to the guiding and locking element 120 and the outer housing 102 by means of engagement of the radially outwardly extending protrusion 424 with opening 232 in the outer plunger portion 132, specifically by means of engagement of forwardly facing surface 430 with a face of opening 230. Additionally, the engagement of radially outwardly extending protrusion 424 with opening 230 restricts rotational displacement of the plunger assembly 130 relative to the guiding and locking element 120 in a counter-clockwise direction, specifically by means of engagement of laterally facing surface 432 with a face of opening 230. It is noted that the rotation direction can be reversed according to another embodiment of the present invention.

It is noted that the guiding teeth 450 of the guiding and locking element 120 are guided along the locking groove portion 344 during relative rotation between the plunger rod assembly 130 and the guiding and locking element 120.

It is a particular feature of an embodiment of the present invention that the displacement of the plunger assembly 130 relative to the guiding and locking element 120 is guided by engagement of the guiding teeth 450 of the guiding and locking element 120 with the guiding groove 340 of the plunger rod inner portion 134.

It is a further particular feature of an embodiment of the present invention that a tactile and audial indication is provided to the user to indicate transitions between different operative orientations during the process of medicament reconstitution, priming and injection of the medicament from the medicament cartridge 140. This indication to the user is provided by engagement of the indicating arms 420 of the guiding and locking element 120 with the guiding tracks 280 of the plunger rod outer portion 132.

It is particularly seen in FIGS. 9D and 9E that the outwardly extending protrusions 424 of the indicating arms 420 are now seated within openings 230 of the plunger rod outer portion 132. It is noted that the indicating arms 420 are relatively resilient and thus are removed from openings 232 upon rotation of the plunger assembly 130, due to the tapered configuration of surface 428 of the indicating arms 420, guided through unlocking groove 292 of the guiding tracks 280 and are now seated within openings 230 of the plunger rod outer portion 132.

It is a particular feature of an embodiment of the present invention, as seen both in FIGS. 9A & 9E, that openings 212 of the plunger rod outer portion 132 are not aligned with windows 172 and 396 anymore, rather demarcation 240, preferably designated as “1” is now aligned with windows 172 and 396, thus providing a visual indication to the user about transitions between different operative orientations during the process of medicament reconstitution, priming and injection of the medicament from the medicament cartridge 140. Specifically, in this unlocked operative orientation shown in FIG. 9A, a visual indication is provided to the user, instructing the user to displace the plunger rod assembly 130 axially forwardly relative to the outer housing 102.

It is a further particular feature of an embodiment of the present invention that an additional visual indication is provided to the user by means of alignment of one of the demarcations 182 of the outer housing 102 with demarcation 220 of the plunger rod outer portion 132. In this unlocked operative orientation, the demarcation 220 is preferably aligned with an unlock sign.

It is additionally noted that the user can inspect the medicament through window 170 of the outer housing 102, which is aligned with window 394 of the guiding and locking element 120, as well as through window 172 of the outer housing 102, which is aligned with window 396 of the guiding and locking element 120.

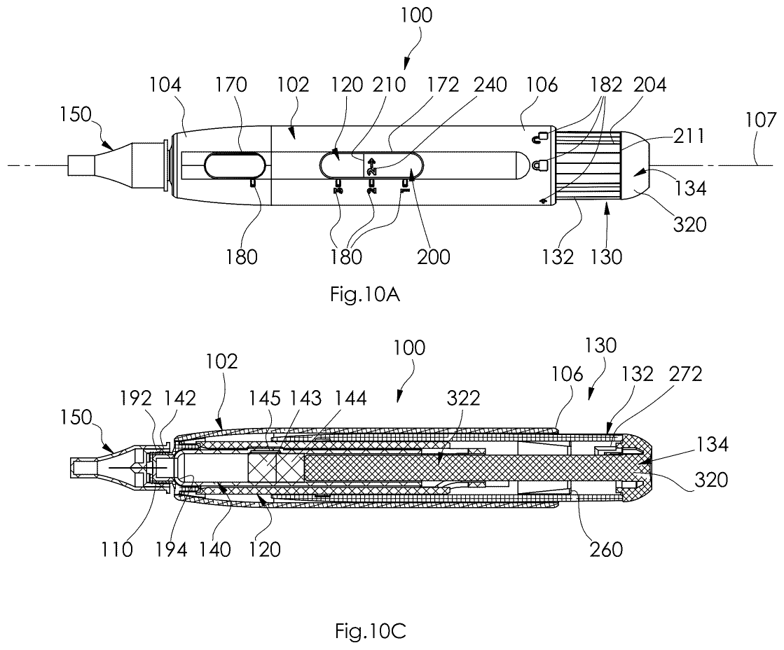

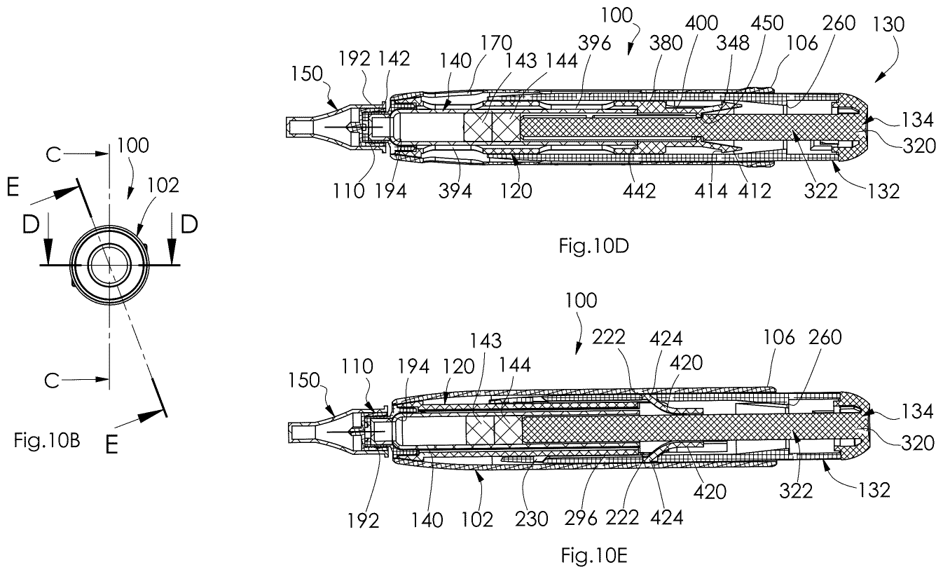

Reference is now made to FIGS. 10A, 10B, 10C, 10D and 10E, which are simplified drawings of the dual chamber injection system 100 of FIGS. 1A-5F in an end of reconstitution and prior to priming operative orientation, including respectively a simplified plan side view, plan top view and three sectional views taken along lines C-C. D-D and E-E in FIG. 10B. Reference is additionally made to FIGS. 10F and 10G, which are simplified drawings of the dual chamber injection system 100 of FIGS. 1A-5F in the end of reconstitution and prior to priming operative orientation, including two different partially cut-out perspective views, FIG. 10F is shown without the outer housing 102 of FIGS. 2A & 2B and FIG. 10G is shown without both the outer housing 102 of FIGS. 2A & 2B and without the plunger rod outer portion 132 of FIGS. 3A-3E.

The dual chamber injection system 100 is seen in FIGS. 10A-10G in the end of reconstitution and prior to priming operative orientation. It is seen particularly in FIGS. 10A-10G that the plunger rod assembly 130 is disposed in the same rotational orientation relative to the outer housing 102 as shown in FIGS. 9A-9F. It is also particularly seen in FIGS. 10A-10G that the plunger rod assembly 130 is forwardly axially displaced along longitudinal axis 107, relative to the outer housing 102, as compared to the unlocked orientation of the dual chamber injection system 100 shown in FIGS. 9A-9F.

It is seen that in this end of reconstitution and prior to priming operative orientation of the dual chamber injection system 100, the guiding portion 400 of the guiding and locking element 120 is less forwardly spaced from locking shoulder 260 of the plunger rod outer portion 132 as compared to the unlocked operative orientation shown in FIGS. 9A-9F.

It is seen that in this end of reconstitution and prior to priming operative orientation that the pistons 143 and 144 now at least partially overlap with the bypass protrusion 145. The rearward piston 144 abuts the forward piston 143 and the plunger rod inner portion 134 engages the rearward piston 144. It is noted that in this end of reconstitution and prior to priming operative orientation the entire amount od diluent previously contained between the two pistons 143 and 144 is transferred into the forward chamber of the medicament cartridge 140 through the bypass protrusion 145. The forward chamber of the medicament cartridge 140 disposed forwardly of forward piston 143 contains the medicament and the dual chamber injection system 100 is now ready to be shaken by the user in order to mix the medicament with the diluent, thus completing the medicament reconstitution process.

It is noted that most of the spatial relationships between the different components of the dual chamber injection system 100 remain the same as described and illustrated with respect to FIGS. 9A-9F, besides the following:

It is a particular feature of an embodiment of the present invention that during forward axial displacement of the plunger rod assembly 130 relative to the outer housing 102, the plunger rod assembly 130 is also axially forwardly displaced relative to the guiding and locking element 120, due to fixed coupling between the guiding and locking element 120 with the outer housing 102. It is particularly seen in FIGS. 10D & 10G that upon forward axial displacement of the plunger rod assembly 130 relative to guiding and locking element 120, the guiding teeth 450 of the guiding and locking element 120 are guided along the reconstitution groove portion 346 of the guiding grooves 340 and are now disposed at the forward end of the helical priming groove portion 348 of guiding grooves 340.

It is appreciated that in this end of reconstitution and prior to priming operative orientation of the dual chamber injection system 100, the plunger assembly 130 is restricted from further forward axial displacement relative to the guiding and locking element 120 due to engagement of the guiding teeth 450 with the priming groove portion 348, which is helically shaped and thus urge rotation of the plunger rod assembly 130 about longitudinal axis 107.

It is also appreciated that the plunger assembly 130 is restricted from rearward axial displacement relative to the guiding and locking element 120 and the outer housing 102 by means of engagement of the radially outwardly extending protrusion 424 with opening 222 in the outer plunger portion 132, specifically by means of engagement of forwardly facing surface 430 with a face of opening 222. Additionally, the engagement of radially outwardly extending protrusion 424 with opening 222 restricts rotational displacement of the plunger assembly 130 relative to the guiding and locking element 120 in a counter-clockwise direction, specifically by means of engagement of laterally facing surface 432 with a face of opening 222. It is noted that the rotation direction can be reversed according to another embodiment of the present invention.

It is noted that the guiding teeth 450 of the guiding and locking element 120 are guided along the reconstitution groove portion 346 during relative axial displacement between the plunger rod assembly 130 and the guiding and locking element 120.

It is a particular feature of an embodiment of the present invention that the displacement of the plunger assembly 130 relative to the guiding and locking element 120 is guided by engagement of the guiding teeth 450 of the guiding and locking element 120 with the guiding groove 340 of the plunger rod inner portion 134.

It is a further particular feature of an embodiment of the present invention that a tactile and audial indication is provided to the user to indicate transitions between different operative orientations during the process of medicament reconstitution, priming and injection of the medicament from the medicament cartridge 140. This indication to the user is provided by engagement of the indicating arms 420 of the guiding and locking element 120 with the guiding tracks 280 of the plunger rod outer portion 132.

It is particularly seen in FIGS. 10E and 10F that the outwardly extending protrusions 424 of the indicating arms 420 are now seated within openings 222 of the plunger rod outer portion 132. It is noted that the indicating arms 420 are relatively resilient and thus are removed from openings 230 upon axial displacement of the plunger assembly 130, due to the tapered configuration of surfaces 426 of the indicating arms 420, guided through reconstitution groove 296 of the guiding tracks 280 and are now seated within openings 222 of the plunger rod outer portion 132.

It is a particular feature of an embodiment of the present invention, as seen both in FIGS. 10A & 10F, that openings 212 of the plunger rod outer portion 132 remain not aligned with windows 172 and 396, rather demarcation 240, preferably designated as “2” is now aligned with windows 172 and 396, thus providing a visual indication to the user about transitions between different operative orientations during the process of medicament reconstitution, priming and injection of the medicament from the medicament cartridge 140. Specifically, in this end of reconstitution prior to priming operative orientation shown in FIG. 10A, a visual indication is provided to the user, instructing the user to rotate the plunger rod assembly 130 about longitudinal axis 107 relative to the outer housing 102.

It is additionally noted that the user can inspect the medicament through window 170 of the outer housing 102, which is aligned with window 394 of the guiding and locking element 120.

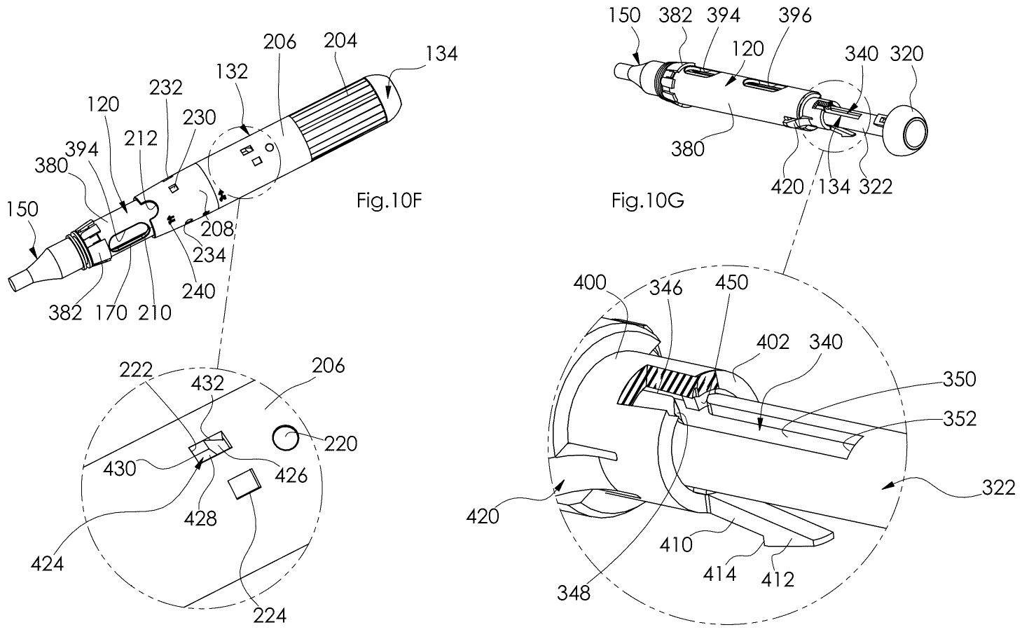

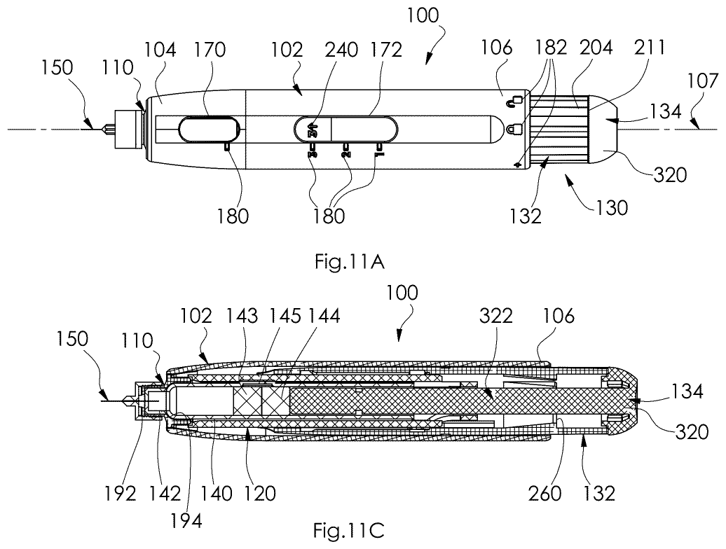

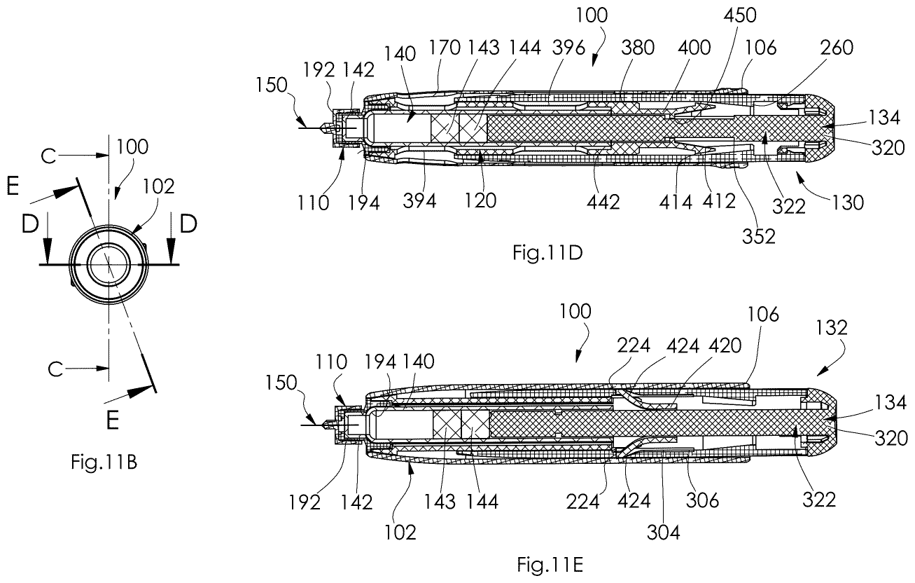

Reference is now made to FIGS. 11A, 11B, 11C, 11D and 11E, which are simplified drawings of the dual chamber injection system 100 of FIGS. 1A-5F in an end of priming and prior to injection operative orientation, including respectively a simplified plan side view, plan top view and three sectional views taken along lines C-C, D-D and E-E in FIG. 11B. Reference is additionally made to FIGS. 11F and 11G, which are simplified drawings of the dual chamber injection system 100 of FIGS. 1A-5F in the end of priming and prior to injection operative orientation, including two different partially cut-out perspective views, FIG. 11F is shown without the outer housing 102 of FIGS. 2A & 2B and FIG. 11G is shown without both the outer housing 102 of FIGS. 2A & 2B and without the plunger rod outer portion 132 of FIGS. 3A-3E.

The dual chamber injection system 100 is seen in FIGS. 11A-11G in the end of priming and prior to injection operative orientation. It is seen particularly in FIGS. 11A-11G that the plunger rod assembly 130 is slightly forwardly axially displaced relative to the outer housing 102 as compared to FIGS. 10A-10G. It is also particularly seen in FIGS. 11A-11G that the plunger rod assembly 130 is rotationally displaced about longitudinal axis 107, relative to the outer housing 102, as compared to the end of reconstitution and prior to priming operative orientation of the dual chamber injection system 100 shown in FIGS. 10A-10G.

It is seen that in this end of priming and prior to injection operative orientation of the dual chamber injection system 100, the guiding portion 400 of the guiding and locking element 120 is less forwardly spaced from locking shoulder 260 of the plunger rod outer portion 132 as compared to the end of reconstitution and prior to injection operative orientation shown in FIGS. 10A-10G.

It is seen that in this end of priming and prior to injection operative orientation that the pistons 143 and 144 now are slightly forwardly axially displaced by the plunger rod assembly 130 to enable priming of the medicament cartridge 140. It is noted that in this end of priming and prior to injection operative orientation, small amount of reconstituted medicament is ejected from the medicament cartridge through the needle, while the needle cover is previously removed to expose the needle. The dual chamber injection system 100 is now ready for injecting the medicament into the injection site.

It is noted that most of the spatial relationships between the different components of the dual chamber injection system 100 remain the same as described and illustrated with respect to FIGS. 10A-10G, besides the following:

It is a particular feature of an embodiment of the present invention that during priming the plunger rod assembly 130 is being both axially forwardly displaced relative to the outer housing 102 and the guiding and locking element 120, and also rotated about longitudinal axis 107 relative to the outer housing 102 and the guiding and locking element 120. It is particularly seen in FIGS. 11D & 11G that upon displacing the plunger rod assembly 130 both forwardly axially and rotatably relative to the outer housing and the guiding and locking element 120, the guiding teeth 450 of the guiding and locking element 120 are guided along the helical priming groove portion 348 of guiding grooves 340 and are now disposed at the forward end of the injection groove portion 350 of guiding grooves 340.

It is appreciated that in this end of priming and prior to injection operative orientation of the dual chamber injection system 100, the plunger assembly 130 is permitted to be displaced axially forwardly along longitudinal axis 107 relative to the guiding and locking element 120 due to engagement of the guiding teeth 450 with the injection groove portion 350, which extend axially along longitudinal axis 107 up to forwardly facing surface 352. Additionally, the engagement of the guiding teeth 450 with the injection groove 350 prevents rotation of the plunger assembly 130 relative to the guiding and locking element 120 in both directions.

It is also appreciated that the plunger assembly 130 is restricted from rearward axial displacement relative to the guiding and locking element 120 and the outer housing 102 by means of engagement of the radially outwardly extending protrusion 424 with opening 224 in the outer plunger portion 132, specifically by means of engagement of forwardly facing surface 430 with a face of opening 224. Additionally, the engagement of radially outwardly extending protrusion 424 with opening 224 restricts rotational displacement of the plunger assembly 130 relative to the guiding and locking element 120 in a counter-clockwise direction, specifically by means of engagement of laterally facing surface 432 with a face of opening 224. It is noted that the rotation direction can be reversed according to another embodiment of the present invention.

It is noted that the guiding teeth 450 of the guiding and locking element 120 are guided along the helical priming groove portion 348 during thread-like displacement between the plunger rod assembly 130 and the guiding and locking element 120.

It is a particular feature of an embodiment of the present invention that the displacement of the plunger assembly 130 relative to the guiding and locking element 120 is guided by engagement of the guiding teeth 450 of the guiding and locking element 120 with the guiding groove 340 of the plunger rod inner portion 134.

It is a further particular feature of an embodiment of the present invention that a tactile and audial indication is provided to the user to indicate transitions between different operative orientations during the process of medicament reconstitution, priming and injection of the medicament from the medicament cartridge 140. This indication to the user is provided by engagement of the indicating arms 420 of the guiding and locking element 120 with the guiding tracks 280 of the plunger rod outer portion 132.

It is particularly seen in FIGS. 11E and 11F that the outwardly extending protrusions 424 of the indicating arms 420 are now seated within openings 224 of the plunger rod outer portion 132. It is noted that the indicating arms 420 are relatively resilient and thus are removed from openings 222 upon axial and rotatable displacement of the plunger assembly 130 defined by the shape of the helical priming groove portion 348, guided through helical priming groove 300 of the guiding tracks 280 and are now seated within openings 224 of the plunger rod outer portion 132.

It is a particular feature of an embodiment of the present invention, as seen both in FIGS. 11A & 11F, that openings 212 of the plunger rod outer portion 132 remain not aligned with windows 172 and 396, rather demarcation 240, preferably designated as “3” is now aligned with windows 172 and 396, thus providing a visual indication to the user about transitions between different operative orientations during the process of medicament reconstitution, priming and injection of the medicament from the medicament cartridge 140. Specifically, in this end of priming and prior to injection operative orientation shown in FIG. 11A, a visual indication is provided to the user, instructing the user to displace the plunger rod assembly 130 axially forwardly along longitudinal axis 107 relative to the outer housing 102.

It is additionally noted that the user can inspect the medicament through window 170 of the outer housing 102, which is aligned with window 394 of the guiding and locking element 120.

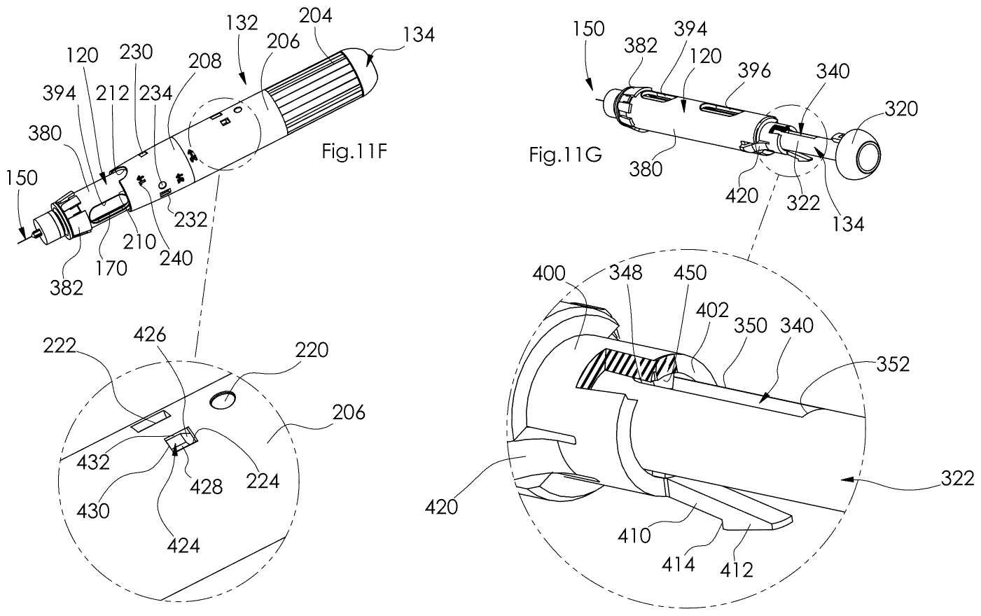

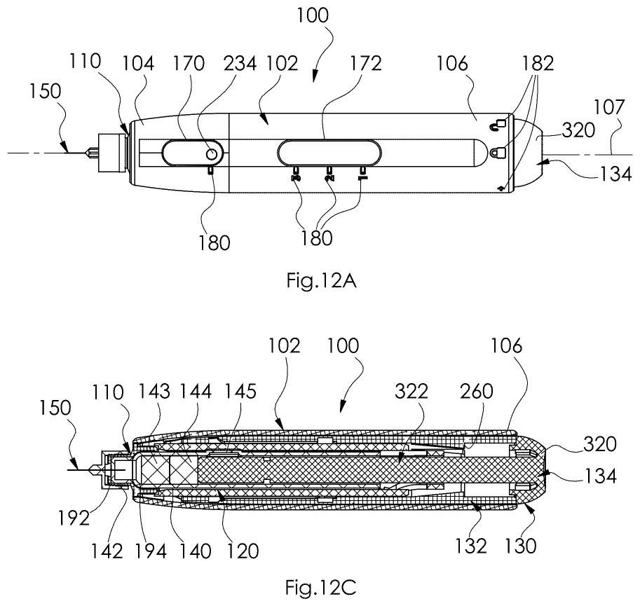

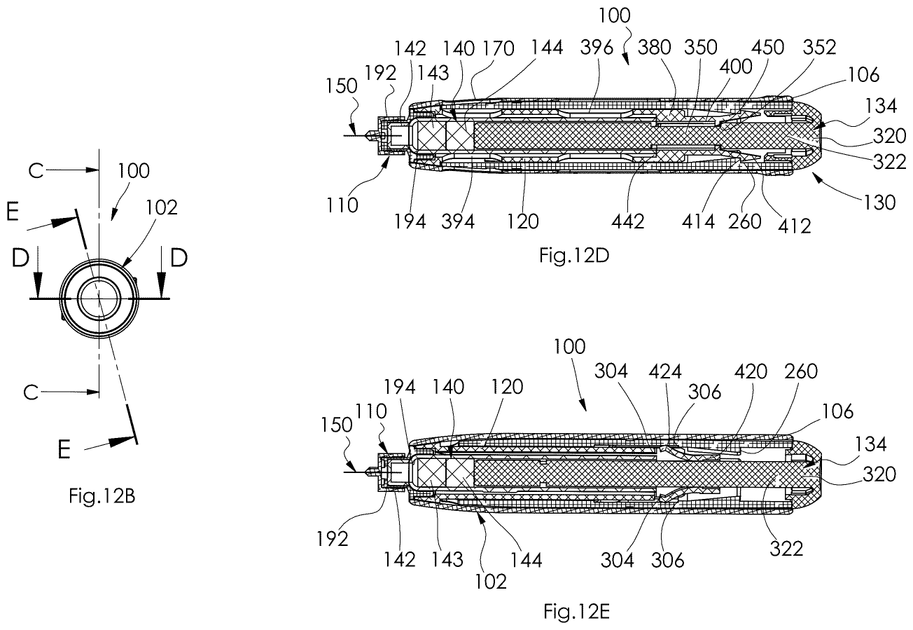

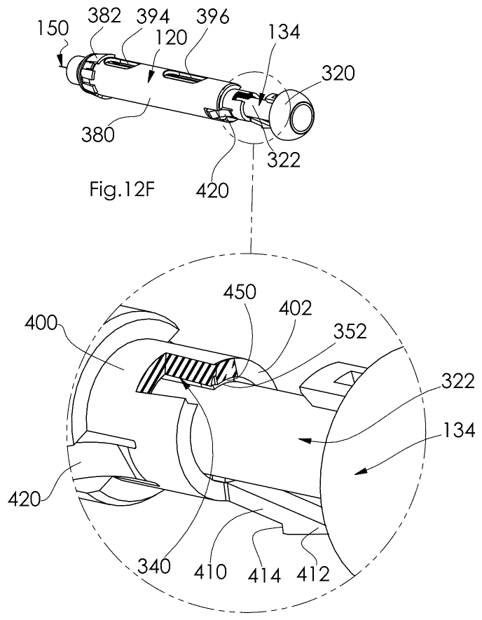

Reference is now made to FIGS. 12A, 12B, 12C, 12D and 12E, which are simplified drawings of the dual chamber injection system 100 of FIGS. 1A-5F in an end of injection locked operative orientation, including respectively a simplified plan side view, plan top view and three sectional views taken along lines C-C, D-D and E-E in FIG. 12B. Reference is additionally made to FIG. 12F, which is a simplified drawing of the dual chamber injection system 100 of FIGS. 1A-5F in the end of injection locked operative orientation, including a partially cut-out perspective view, shown without both the outer housing 102 of FIGS. 2A & 2B and without the plunger rod outer portion 132 of FIGS. 3A-3E.

The dual chamber injection system 100 is seen in FIGS. 12A-12F in the end of injection operative orientation. It is seen particularly in FIGS. 12A-12F that the plunger rod assembly 130 is fully forwardly axially displaced relative to the outer housing 102.

It is seen that in this end of injection operative orientation of the dual chamber injection system 100, the lateral finger protrusions 412 of the guiding and locking element 120 are rearwardly disposed relative to the locking shoulder 260 of the plunger rod outer portion 132, such that forwardly facing locking surface 414 of the lateral finger protrusions 412 are rearwardly supported against locking shoulder 260 and thereby prevent axial rearward displacement of the plunger assembly 130 relative to the guiding and locking element 120 and thus relative to the outer housing 102 in this end of injection operative orientation shown in FIGS. 12A-12F, in which the plunger assembly 130 is locked relative to both the guiding and locking element 120 and to the outer housing 102.

It is noted that in another embodiment of the present invention, each of the guiding tracks 280 may have an additional recess adjacent the forwardly facing shoulder 306 thereof, so that the indicating arms 420 are engaged with these recesses in this end of injection locked operative orientation in order to provide a visual and/or tactile indication of the end of injection locked operative orientation to the user. This engagement of the indicating arms 420 with the recesses also preferably restricts rearward displacement of the plunger assembly 130 relative to the guiding and locking element 120. Additionally, the engagement of the indicating arms 420 with the recesses also preferably restricts rotational displacement of the plunger assembly 130 relative to the guiding and locking element 120 generally in a counterclockwise direction. It is noted that the direction of rotation can be reversed in accordance with another embodiment of the present invention.

It is seen that in this end of injection operative orientation that the pistons 143 and 144 are now forwardly axially displaced by the plunger rod assembly 130 up to the forward end of the medicament cartridge 140 to enable injection of the entire amount of medicament from the medicament cartridge 140. The dual chamber injection system 100 is now at the end of medicament delivery locked operative orientation and is ready for disposal.

It is noted that most of the spatial relationships between the different components of the dual chamber injection system 100 remain the same as described and illustrated with respect to FIGS. 11A-11G, besides the following:

It is a particular feature of an embodiment of the present invention that during injection, the plunger rod assembly 130 is being axially forwardly displaced relative to the outer housing 102 and the guiding and locking element 120. It is particularly seen in FIGS. 12D & 12F that upon displacing the plunger rod assembly 130 forwardly axially relative to the outer housing 102 and the guiding and locking element 120, the guiding teeth 450 of the guiding and locking element 120 are guided along the injection groove portion 350 of guiding grooves 340 and are now disposed adjacent to the forwardly facing surface 352 of the injection groove portion 350 of guiding grooves 340.

It is appreciated that in this end of injection operative orientation of the dual chamber injection system 100, the plunger assembly 130 is locked and restricted from forward axial displacement and from rotation by means of engagement between the guiding teeth 450 of the guiding and locking element 120 and the forwardly facing surface 352 of the guiding grooves 340. The plunger assembly 130 is further restricted from rearward axial displacement by means of engagement between the lateral finger protrusions 412 and the locking shoulder 260 of the outer plunger portion 132.

It is noted that the guiding teeth 450 of the guiding and locking element 120 are guided along the injection groove portion 350 during forward axial displacement of the plunger rod assembly 130 relative to the guiding and locking element 120 and the outer housing 102.

It is a particular feature of an embodiment of the present invention that the displacement of the plunger assembly 130 relative to the guiding and locking element 120 is guided by engagement of the guiding teeth 450 of the guiding and locking element 120 with the guiding groove 340 of the plunger rod inner portion 134.

It is a further particular feature of an embodiment of the present invention that a tactile and audial indication is provided to the user to indicate transitions between different operative orientations during the process of medicament reconstitution, priming and injection of the medicament from the medicament cartridge 140. This indication to the user is provided by engagement of the indicating arms 420 of the guiding and locking element 120 with the guiding tracks 280 of the plunger rod outer portion 132.

It is particularly seen in FIG. 12E that the outwardly extending protrusions 424 of the indicating arms 420 are now disposed adjacent to and are preferably supported against forwardly facing shoulders 306 of the plunger rod outer portion 132. It is noted that the indicating arms 420 are relatively resilient and thus are removed from openings 224 upon axial displacement of the plunger assembly 130, due to the tapered configuration of surface 426, and guided through injection groove 304 of the guiding tracks 280 up to engagement with the forwardly facing shoulders 306 of the plunger rod outer portion 132.

It is a particular feature of an embodiment of the present invention, as seen in FIG. 12A, that demarcation 234 of the outer plunger portion 132 is now aligned with demarcation 180 disposed adjacent to window 170 of the outer housing 102, thus providing a visual indication of end of injection to the user.

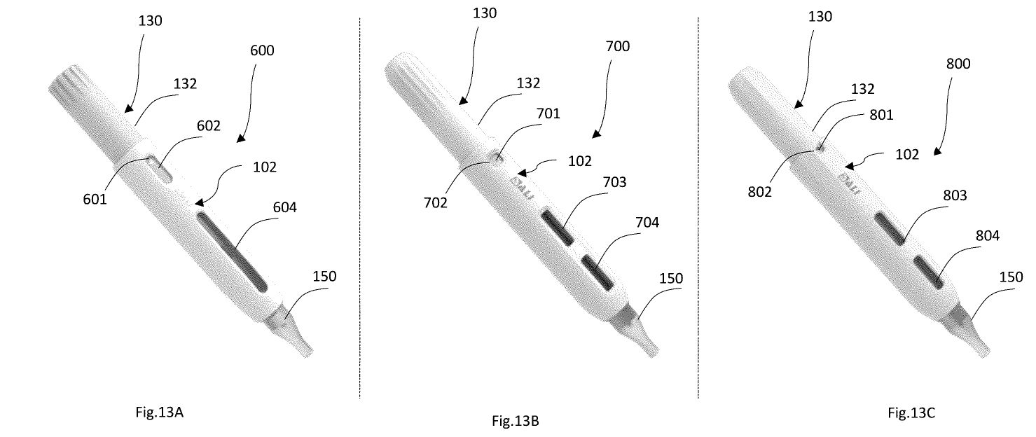

Reference is now made to FIGS. 13A-13C, which are simplified drawings of three different embodiments of the assembled dual chamber injection systems.

Turning specifically to FIG. 13A, a dual chamber injection system 600 is illustrated in accordance with another embodiment of the present invention. It is seen in FIG. 13A that the demarcations that are described in detail hereinabove are provided in form of a printed demarcation 601 on the outer plunger portion 132. A longitudinal window 602 is provided on the outer housing 102, which corresponds with the printed demarcation 601 provided on the outer plunger portion 132. It is noted that the printed demarcation 601 changes its position within the longitudinal window 602 along with displacement of the plunger assembly 130 relative to the outer housing 102, and thus provides a visual indication to the user as to the different operative orientations of the dual chamber injection system 600.

It is additionally seen in FIG. 13A that a single longitudinal indicating window 604 is formed at a forward end of the outer housing 102, instead of the two windows 170 and 172 in the embodiment illustrated in FIGS. 1A-12F.

Turning specifically to FIG. 13B, a dual chamber injection system 700 is illustrated in accordance with still another embodiment of the present invention. It is seen in FIG. 13B that the demarcations that are described in detail hereinabove are provided in form of a printed demarcation 701 on the outer plunger portion 132. A window 702 is provided on the outer housing 102, which corresponds with the printed demarcation 701 provided on the outer plunger portion 132. It is noted that the printed demarcation 701 changes its position relative to the window 702 along with displacement of the plunger assembly 130 relative to the outer housing 102, and thus provides a visual indication to the user as to the different operative orientations of the dual chamber injection system 700.

It is additionally seen in FIG. 13B that two windows 703 and 704 are formed at a forward end of the outer housing 102, similarly to the two windows 170 and 172 in the embodiment illustrated in FIGS. 1A-12F.

Turning specifically to FIG. 13C, a dual chamber injection system 800 is illustrated in accordance with yet another embodiment of the present invention. It is seen in FIG. 13C that the demarcations that are described in detail hereinabove are provided in form of a printed demarcation 801 on the outer plunger portion 132. A recess 802 is formed at a rearward end of the outer housing 102, which corresponds with the printed demarcation 801 provided on the outer plunger portion 132. It is noted that the printed demarcation 801 changes its position relative to the recess 802 along with displacement of the plunger assembly 130 relative to the outer housing 102, and thus provides a visual indication to the user as to the different operative orientations of the dual chamber injection system 800.

It is additionally seen in FIG. 13C that two windows 803 and 804 are formed at a forward end of the outer housing 102, similarly to the two windows 170 and 172 in the embodiment illustrated in FIGS. 1A-12F.

It is appreciated that any other type of demarcation provided on the plunger assembly 130 and corresponding with any other type of recess or opening provided on the outer housing 102 or vise-versa is considered to be within the scope of the embodiments of the present invention.

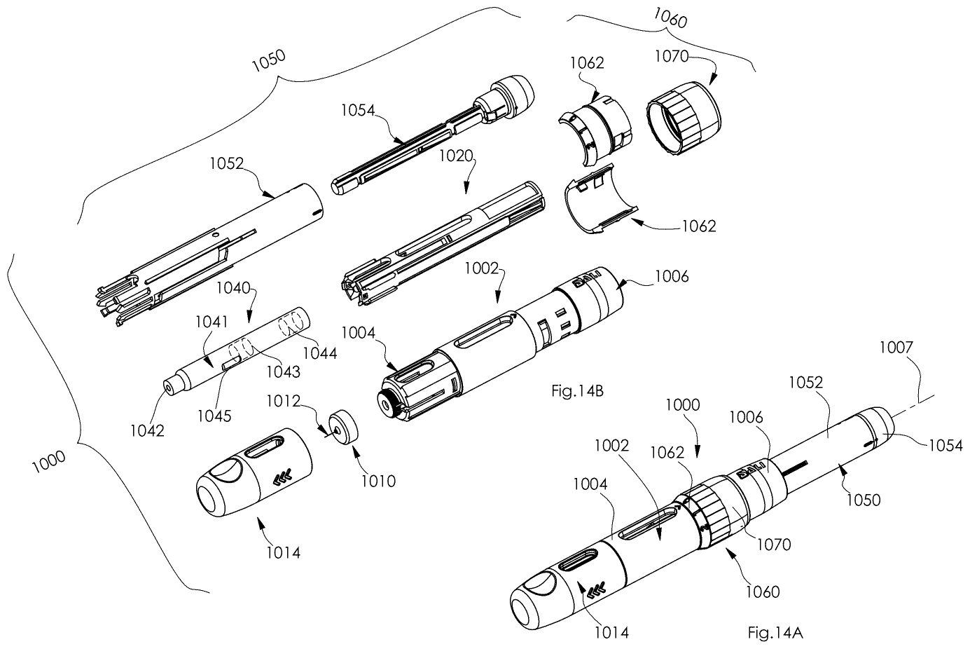

Reference is now made to FIGS. 14A and 14B, which are respectively simplified pictorial view and exploded view of a dual chamber injection system constructed and operative in accordance with another embodiment of the present invention.

As seen in FIGS. 14A & 14B, a dual chamber injection system 1000 preferably includes an outer housing 1002 having a forward end 1004 and a rearward end 1006 and is arranged along a longitudinal axis 1007. A needle hub 1010 with a needle 1012 is configured to be fixedly coupled to the forward end 1004 of the outer housing 1002 or integrally made therewith. A protective cap 1014 is configured to cover the forward end 1004 of the outer housing 1002 and the needle 1012 in certain operative orientations of the dual chamber injection system 1000. A syringe holder 1020 is configured to be at least partially received into the outer housing 1002 and is preferably slidably attached to the outer housing 1002 in certain operative orientations of the dual chamber injection system 1000. It is noted that the syringe holder 1020 is also referred to as a guiding element throughout the description of the embodiments of the present invention.

It is noted that a dual chamber medicament cartridge 1040 is adapted to be inserted into the syringe holder 1020 and fixedly held therein.

It is appreciated that the dual chamber medicament cartridge 1040 can be any type of conventional cartridge, such as a cartridge commercially available from Schott Pharmaceutical Systems, Mainz, Germany or Vetter Pharma International USA Inc., IL, USA or Nuova Ompi S.r.l., Padua, Italy or may be any other suitable syringe or cartridge.

The dual chamber medicament cartridge 1040 preferably includes a cartridge barrel 1041 and a septum 1042 at a forward end thereof. Preferably, two pistons, namely a forward piston 1043 and a rearward piston 1044 are contained within the cartridge barrel 1041 and are adapted for slidable axial displacement relative thereto. It is appreciated that a drug preparation is preferably confined between the forward piston 1043 and the septum 1042 and a solvent is preferably confined between the forward piston 1043 and the rearward piston 1044 and upon appropriate longitudinal displacement of the pistons, the two substances are configured for reconstitution and subsequent ejection, as described in detail hereinbelow.

The cartridge barrel 1041 has a generally cylindrical shape and extends along the longitudinal axis 1007. At least one bypass protrusion 1045 is disposed generally at an intermediate location of the cartridge barrel 1041. The bypass protrusion 1045 generally extends radially outwardly from an outer surface of the cartridge barrel 1041 to facilitate fluid passage between the two chambers formed within the cartridge barrel 1041, one between the two pistons 1043 and 1044 and another one between the forward piston 1043 and the septum 1042.

A plunger rod assembly 1050 is configured to be partially inserted into the rearward end 1006 of the outer housing 1002 and adapted to be axially moveable relative to the outer housing 1002 and relative to the syringe holder 1020 along longitudinal axis 1007. The plunger rod assembly 1050 preferably includes a plunger rod outer portion 1052 and a plunger rod inner portion 1054, which are fixedly attached to each other. Alternatively, the plunger rod outer portion 1052 and the plunger rod inner portion 1054 may be integrally made as a single component.

In accordance with an embodiment of the present invention, the plunger rod inner plunger portion 1054 is at least partially received into the plunger rod outer portion 1052 and arranged coaxially therewith along the longitudinal axis 1007.

A ring assembly 1060 is preferably rotatably mounted over a portion of the outer housing 1002. Typically, two generally identical ring portions 1062 are mounted over a portion of the outer housing 1002 and are preferably encircled and held in place by a collar 1070. It is noted that a single ring can be employed instead of the two ring portions 1062 and the collar 1070 in accordance with another embodiment of the present invention.

It is a particular feature of an embodiment of the present invention that the outer housing 1002, the plunger assembly 1050, the syringe holder 1020 and the ring assembly 1060 are operatively associated with each other to enable penetration of the septum 1042 of the cartridge 1040, reconstitution of medicament, priming of the injector and injection of the medicament into the injection site.

It is specifically noted that the ring assembly 1060 is rotatable relative to the outer housing 1002 about the longitudinal axis 1007, thereby facilitating transitions between the different operative orientations of the dual chamber injection system 1000, resulting in alignment of corresponding indication features disposed on the outer housing 1002 and the ring assembly 1060, thereby enable indicating to the user what is the operative state that the dual chamber injection system 1000 is situated in. Additionally, rotatable displacement of the ring assembly 1060 relative to the outer housing 1002 is configured to either enable or cause axial displacement of the plunger assembly 1050 relative to the outer housing 1002 and syringe holder 1020.

It is further specifically noted that the plunger rod assembly 1050 is axially displaceable relative to the outer housing 1002 and relative to the syringe holder 1020 along longitudinal axis 1007, thereby facilitating transitions between the different operative orientations of the dual chamber injection system 1000. At least in some of the operative orientations of the dual chamber injection system, this axial displacement of the plunger assembly 1050 relative to the outer housing 1002 and the syringe holder 1020 results in displacement of at least one of the pistons 1043 and 1044 relative to cartridge 1040, thereby enabling reconstitution of medicament, priming of the injector and injection of medicament in different operative orientations of the dual chamber injection system 1000.

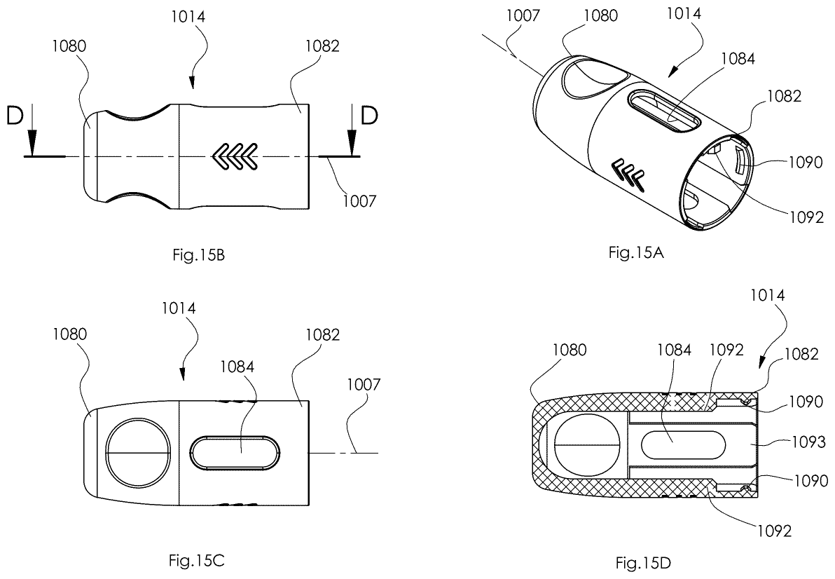

Reference is now made to FIGS. 15A-15D, which are respectively a simplified pictorial view, two different planar side views and a sectional view taken along lines D-D in FIG. 15B of the protective cap 1014 forming part of the dual chamber injection system 100 of FIGS. 14A & 14B.

In accordance with an embodiment of the present invention, the protective cap 1014 is an integrally formed element, preferably injection molded of plastic and is arranged along longitudinal axis of symmetry 1007.

The protective cap 1014 is a generally longitudinal hollow cylindrical element having a closed forward end 1080 and an open rearward end 1082. Preferably, a window 1084 is formed adjacent the rearward end 1082 thereof.

Preferably, a plurality of gripping surfaces are preferably provided on an outer surface of the protective cap 1014 and a plurality of demarcations are preferably provided on the outer surface of the protective cap 1014 to illustrate the direction of cap removal.

It is specifically seen in FIGS. 15A and 15D that several protrusions 1090 are formed on an inner surface of the protective cap 1014, preferably disposed adjacent the rearward end 1082 thereof and extending radially inwardly.

It is further specifically seen in FIG. 15D that several longitudinal ribs 1092 are formed on the inner surface of the protective cap 1014. The ribs 1092 extend radially inwardly and are radially spaced from each other. The ribs 1092 are preferably located forwardly to the protrusions 1090.

It is also seen that a longitudinal recessed surface 1093 is formed between each of the ribs 1092.

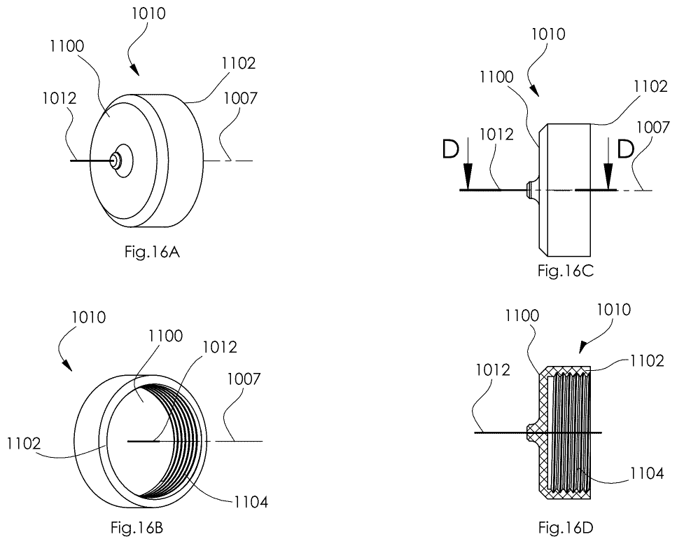

Reference is now made to FIGS. 16A-16D, which are respectively two different simplified pictorial views, planar side view and a sectional view taken along lines D-D in FIG. 16C of the needle hub 1010 forming part of the dual chamber injection system 100 of FIGS. 14A & 14B.

In accordance with an embodiment of the present invention, the needle hub 1010 is an integrally formed element, preferably injection molded of plastic and is arranged along longitudinal axis of symmetry 1007.

Needle 1012 is preferably fixedly attached to the needle hub 1010.

It is seen in FIGS. 16A-16D that the needle hub 1010 preferably has a closed forward end 1100 and a rearward circumferential edge 1102. Preferably, the needle hub 1010 has an internal thread 1104 for connection thereof to the outer housing 1002.

It is noted that the needle hub 1010 can alternatively be connected to the outer housing 1002 using any other suitable connection means or can further alternatively be integrally made therewith.

It is noted that alternatively a needle assembly may be incorporated into the dual chamber injection system 1000. The needle assembly can be similar to the automatic needle that is described in U.S. Pat. No. 9,844,634 or U.S. Pub. 20190125978, which are incorporated by reference herein in their entirety. The needle assembly may be configured for automatic shielding, automatic needle penetration or both.

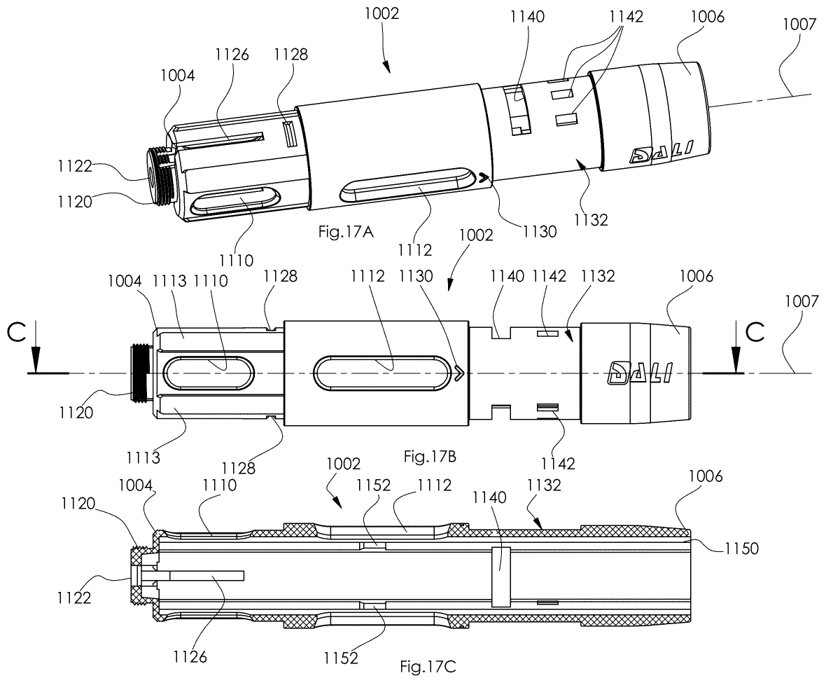

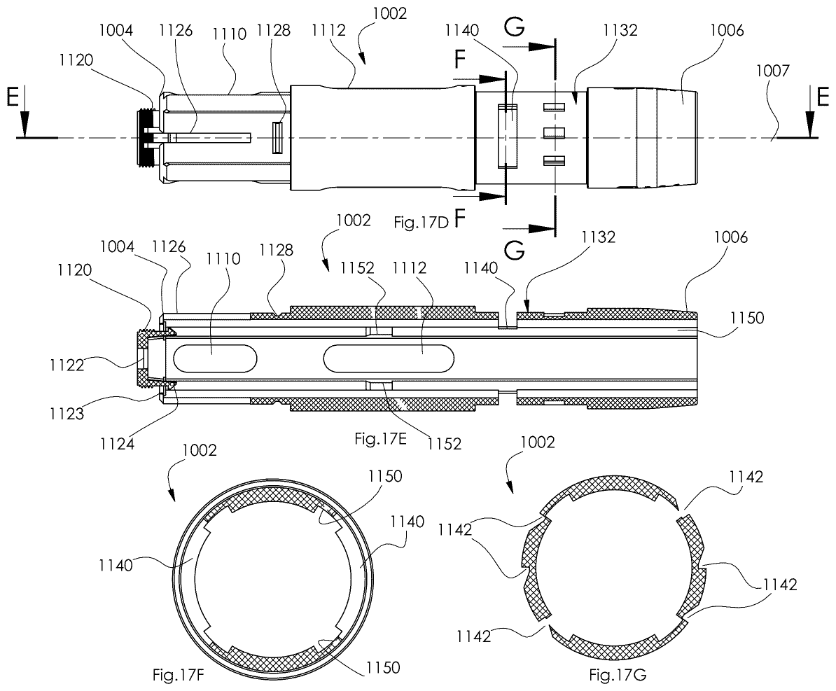

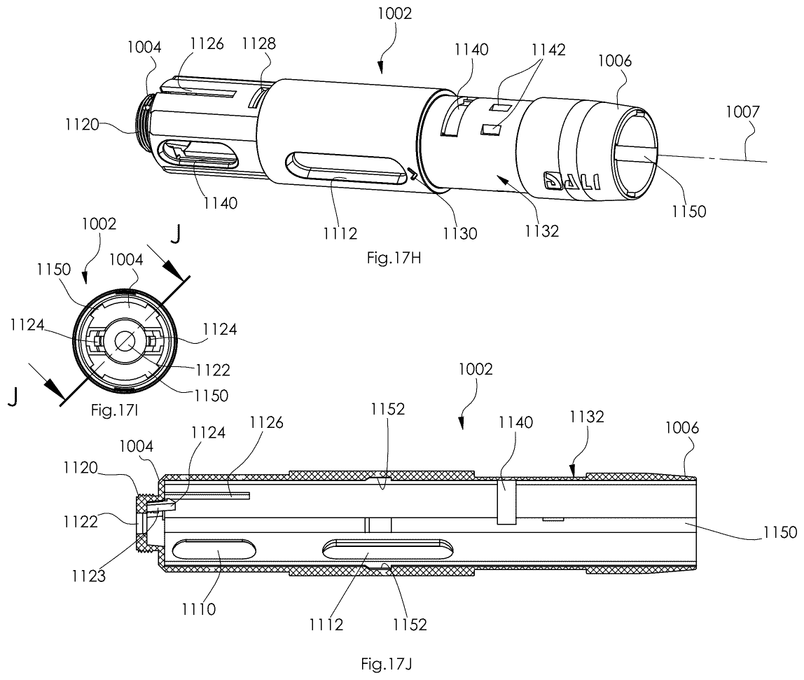

Reference is now made to FIGS. 17A, 17B, 17C, 17D, 17E, 17F, 17G, 17H, 17I and 17J, which are respectively a simplified pictorial view, planar side view, sectional view taken along lines C-C in FIG. 17B, a different planar side view, three sectional views taken along lines E-E, F-F and G-G in FIG. 17D, a different pictorial view, a planar top view and sectional view taken along lines J-J in FIG. 17I of the outer housing 1002 forming part of the dual chamber injection system 1000 of FIGS. 14A & 14B.

In accordance with an embodiment of the present invention, the outer housing 1002 is an integrally formed element, preferably injection molded of plastic and is arranged along longitudinal axis of symmetry 1007.

The outer housing 1002 is a generally longitudinal hollow cylindrical element having forward end 1004 and rearward end 1006. Preferably, a forward window 1110 is formed adjacent the forward end 1004 thereof and rearward window 1112 is formed rearwardly of the forward window 1110.

It is seen in FIGS. 17A & 17B that four longitudinal protrusions 1113 are formed on the outer surface of the outer housing 1002 and generally bound the forward window 1110.

An externally threaded protrusion 1120 extends forwardly from the forward end 1004 of the outer housing 1002. A through bore 1122 extends through the externally threaded protrusion 1120. A snap portion 1123 is formed within the externally threaded protrusion 1120 and extends rearwardly therefrom and slightly rearwardly of the forward end 1004. The snap portion 1123 has a radially outwardly extending tooth 1124 disposed slightly rearwardly of the forward end 1004.

Typically, two mutually diametrically opposed grooves 1126 extend rearwardly from the forward end 1004 of the outer housing 1002 and a recess 1128 is disposed rearwardly of at least one of the grooves 1126.

Preferably, a demarcation 1130 is provided rearwardly of the rearward window 1112.

Typically, a recessed portion 1132 is formed rearwardly of the rearward window 1112. having an outer diameter that is preferably smaller than the outer dimeter of the remaining longitudinal extent of the outer housing 1002. The recessed portion 1132 is adapted for mounting of the ring assembly 1060 thereon.

It is particularly seen in FIGS. 17D & 17F that several through openings 1140 are formed at the forward portion of the recessed area 1132, which communicate with the inner volume of the outer housing 1002. Typically, three recesses 1142 are formed in the recessed area 1132, rearwardly of the opening 1140.

It is particularly seen in FIG. 17H that, typically, four grooves 1150 are formed on the inner surface of the outer housing 1002. The grooves 1150 generally longitudinally extend forwardly from the rearward end 1006, adapted to be operatively associated with the plunger rod outer portion 1052.

It is further particularly seen in FIG. 17J that several radially spaced recesses 1152 are formed within each one of the grooves 1150 and are disposed forwardly of openings 1140. The recesses 1152 are adapted to be operatively associated with the plunger rod outer portion 1052 as described in detail hereinbelow.

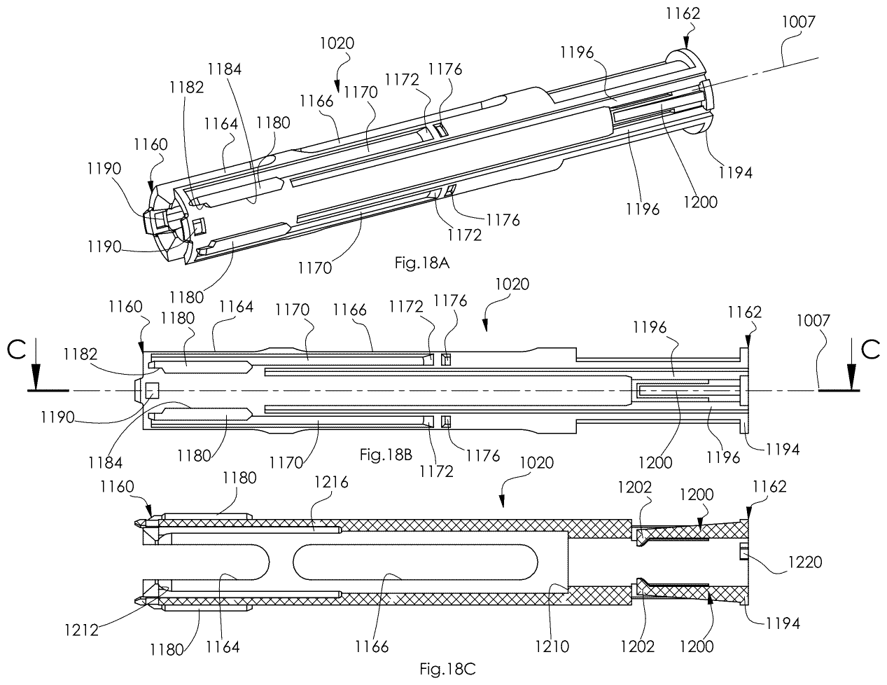

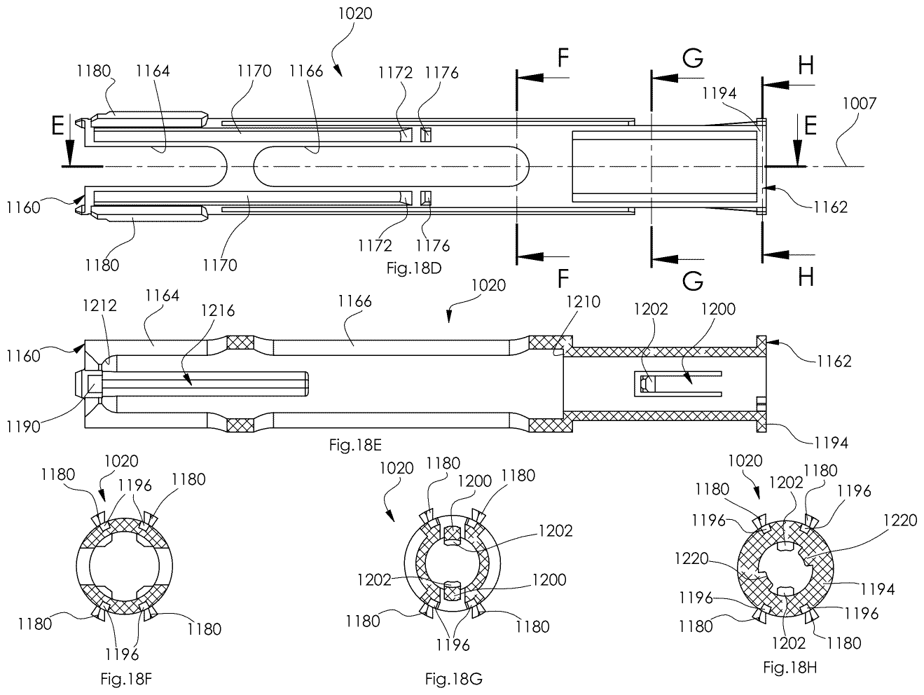

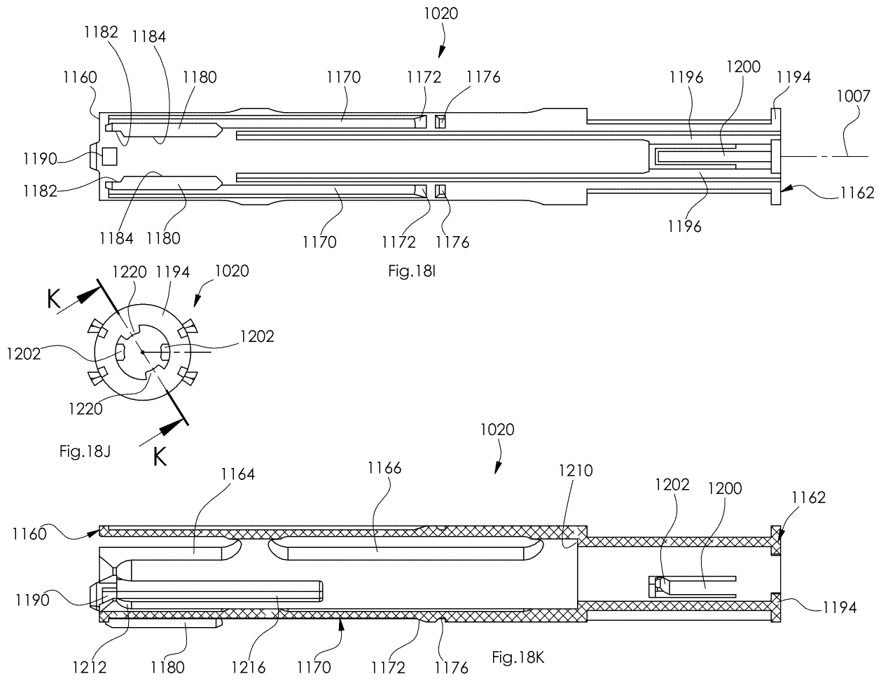

Reference is now made to FIGS. 18A, 18B, 18C, 18D, 18E, 18F, 18G, 18H, 18I, 18J and 18K, which are respectively a simplified pictorial view, planar side view, sectional view taken along lines C-C in FIG. 18B, a different planar side view, four sectional views taken along lines E-E, F-F, G-G and H-H in FIG. 18D, another different planar side view, a planar top view and sectional view taken along lines K-K in FIG. 18J of the syringe holder 1020 forming part of the dual chamber injection system 100 of FIGS. 14A & 14B.

In accordance with an embodiment of the present invention, the syringe holder 1020 is an integrally formed element, preferably injection molded of plastic and is arranged along longitudinal axis of symmetry 1007.

The syringe holder 1020 is a generally longitudinal hollow cylindrical element having forward end 1160 and rearward end 1162. Preferably, a forward window 1164 is formed adjacent the forward end 1160 thereof and rearward window 1166 is formed rearwardly of the forward window 1164.

A plurality of grooves 1170 are formed on the outer circumference of the syringe holder 1020. The grooves 1170 are radially spaced from each other and extend rearwardly from a location that is generally adjacent to the forward end 1160. Each of the grooves 1170 preferably terminates at a rearwardly tapered surface 1172 at the rearward end thereof. A recess 1176 is disposed rearwardly to each of the grooves 1170 and is slightly rearwardly spaced from the rearwardly tapered surface 1172.

Typically, two protrusions 1180 are disposed generally between a pair of grooves 1170. The protrusions 1180 are radially spaced from each other, each one of the protrusions 1180 extends rearwardly from a location adjacent to forward end 1160 to a longitudinal extent generally smaller than the longitudinal extent of the grooves 1170. The protrusions 1180 include a forward inward wall portion 1182 and a rearward inward wall portion 1184. The gap between the forward inward wall portions 1182 of two protrusions 1180 is generally larger than the gap between the rearward inward wall portions 1184 of two protrusions 1180.

It is seen in FIGS. 18A-18K that an opening 1190 is formed generally between two forward inward wall portions 1182 of two protrusions 1180. The opening 1190 is disposed adjacent to the forward end 1160.

A generally circular flange 1194 is formed at the rearward end 1162 of the syringe holder 1020. A plurality of longitudinal grooves 1196 extend from the rearward end 1162 forwardly. The longitudinal grooves 1196 are generally disposed radially inwardly relative to a pair of grooves 1170 and generally overlap therewith a long a portion of their longitudinal extent. The longitudinal grooves 1196 extend to the vicinity of protrusions 1180.

It is particularly seen in FIGS. 18A and 18C that snap portions 1200 are formed at the rearward end 1162 of the syringe holder 1020 and are disposed between a pair of longitudinal grooves 1196. The snap portions 1200 are configured to be radially outwardly deflectable upon application of force thereon. The snap portions 1200 include a radially inwardly directed tooth 1202, as specifically seen in FIG. 18C.

It is also particularly seen in FIG. 18C that a forwardly facing shoulder 1210 is formed forwardly of the radially inwardly directed tooth 1202 and is adapted to support the rearward end of the cartridge 1040. A generally curved forward surface 1212 is formed adjacent the forward end 1160 of the syringe holder 1020 and is adapted to support the forward end of the cartridge 1040.

It is particularly seen in FIG. 18E that a groove 1216 is formed on the inner surface of the syringe holder 1020 and extends rearwardly from a location adjacent opening 1190. The groove 1216 is adapted for passage of the bypass protrusion 1045 of the cartridge 1040 there along.

It is seen in FIG. 18H that protrusions 1220 are formed on the inner surface of the syringe holder 1020. The protrusions 1220 are located adjacent the rearward end 1162 of the syringe holder 1020 and are radially spaced from the snap portions 1200.

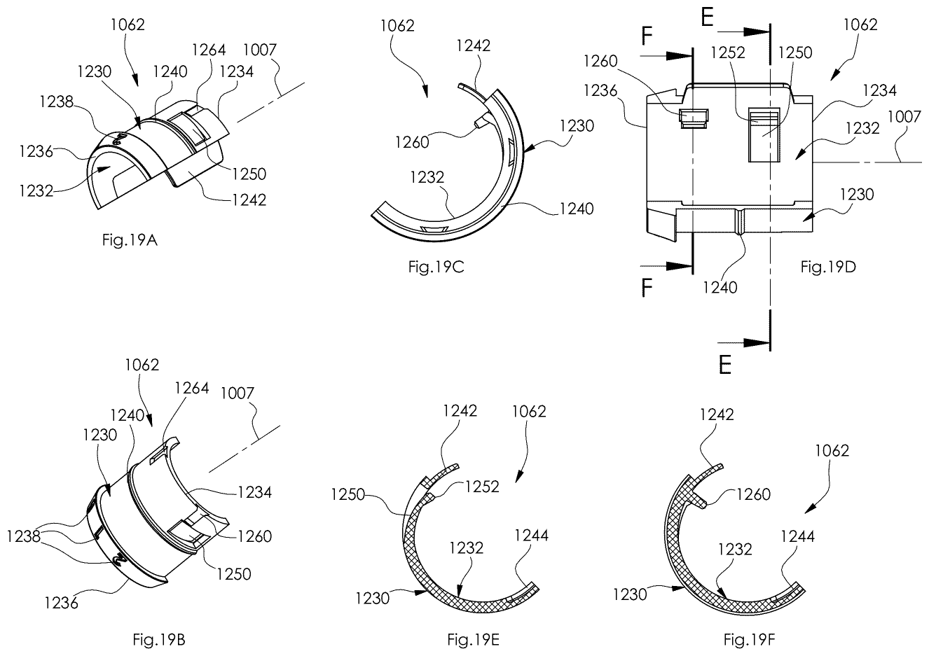

Reference is now made to FIGS. 19A, 19B, 19C, 19D, 19E and 19F, which are respectively two different simplified pictorial views, a top planar view, a side planar view, and two sectional views taken along lines E-E and F-F in FIG. 19D of the ring portion 1062 forming part of the dual chamber injection system 100 of FIGS. 14A & 14B.

In accordance with an embodiment of the present invention, the dual chamber injection system 1000 preferably includes two identical ring portions 1062 that are connected together using a collar 1070 to form a ring assembly 1060. The ring assembly 1060 can be manufactured as a single integral element, but for the purposes of manufacturing, it is divided into several elements, in accordance with an embodiment of the present invention.

For clarity, only one half of the ring is described and illustrated in FIGS. 19A-19F.

The ring portion 1062 is an integrally formed element, preferably injection molded of plastic and is arranged about longitudinal axis of symmetry 1007.

The ring portion 1062 is preferably semi-circular and defines an outer surface 1230 and an inner surface 1232.

The ring portion 1062 has a rearward circumferential edge 1234 and a forward circumferential edge 1236. A set of demarcations 1238 is provided on the outer surface 1230 of the ring portion 1062 and located preferably adjacent the forward circumferential edge 1236 thereof. The set of demarcations 1238 may preferably include a “lock” sign, position “1” and position “2”, each indicating a different operative orientation of the dual chamber injection system 1000.

A circumferential protrusion 1240 is formed on the outer surface 1230 of the ring portion 1062 and adapted to be coupled with the collar 1070.

Ring portion 1062 preferably has a protrusion 1242 that is adapted to be coupled with a recess 1244 formed on the inner surface 1232 of the corresponding second identical ring portion 1062 in order to connect the two identical ring portions 1062 together.

It is seen particularly in FIGS. 19D and 19E that a snap portion 1250 is formed between the protrusion 1240 and the rearward edge 1234 of the ring portion 1062. The snap portion 1250 has a radially inwardly extending finger 1252, adapted for engagement with the outer housing 1002.

It is seen particularly in FIGS. 19D and 19F that a radially inwardly extending tooth 1260 is formed on the inner surface 1232 of the ring portion 1062 and is disposed between the protrusion 1240 and the forward edge 1236 of the ring portion 1062. The radially inwardly extending tooth 1260 is adapted for engagement with the plunger rod outer portion 1052.

A series of radially spaced grooves 1264 are formed adjacent the rearward edge 1234 of the ring portion 1062, adapted to engage a portion of the collar 1070 and prevent rotation thereof relative to the ring portions 1062.

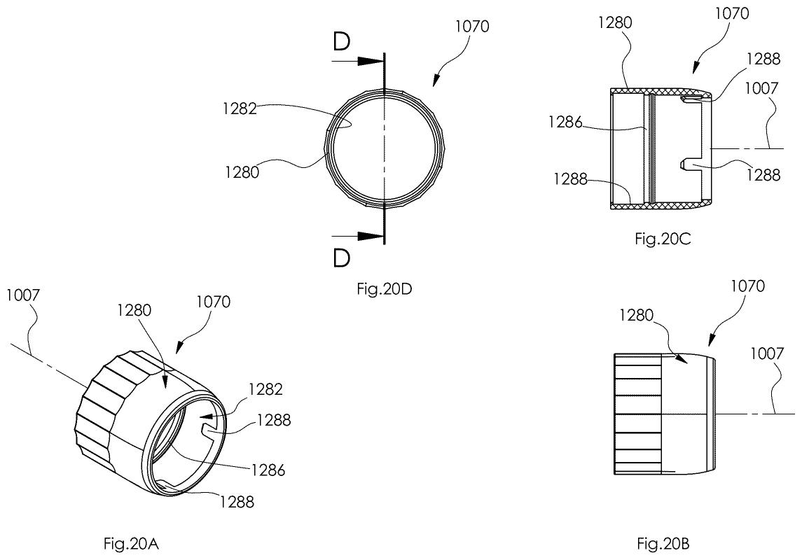

Reference is now made to FIGS. 20A-20D, which are respectively a simplified pictorial view, a side planar view, a top planar view and a sectional view taken along lines D-D in FIG. 20C of the collar 1070 forming part of the dual chamber injection system 1000 of FIGS. 14A & 14B.

In accordance with an embodiment of the present invention, the collar is adapted to be mounted over two identical ring portions 1062 that are connected to each other and hold the ring portions 1062 together, the ring portions 1062 and the collar 1070 together act as one integral component, as the ring assembly 1060.

The collar 1070 is an integrally formed element, preferably injection molded of plastic and is arranged about longitudinal axis of symmetry 1007.

The collar 1070 is preferably a hollow cylindrical element, that defines an outer surface 1280 and an inner surface 1282.

The collar 1070 preferably includes a circumferential groove 1286 formed on the inner surface 1282 thereof and adapted to be coupled to the ring portions 1062. A series of ribs 1288 are formed on the inner surface 1282 of the collar 1070 and adapted to be coupled with the ring portions 1062 to prevent relative rotation between the ring portions 1062 and the collar 1070.

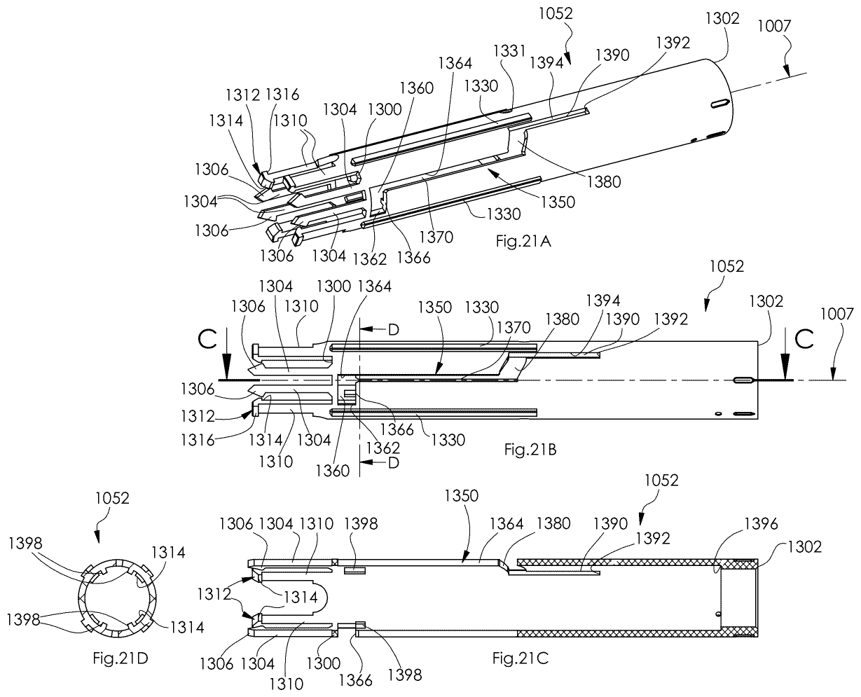

Reference is now made to FIGS. 21A-21D, which are respectively a simplified pictorial view, a planar side view and two simplified sectional views taken along lines C-C and D-D in FIG. 21B of the plunger rod outer portion 1052 forming part of the dual chamber injection system 100 of FIGS. 14A & 14B.

In accordance with an embodiment of the present invention, the plunger rod outer portion 1052 is an integrally formed element, preferably injection molded of plastic and is arranged along longitudinal axis of symmetry 1007.

The plunger rod outer portion 1052 is a generally longitudinal hollow cylindrical element having forward end 1300 and rearward end 1302.

Typically, two pairs of forwardly extending arms 1304 are disposed mutually opposite to each other and extend forwardly from the forward end 1300 of the plunger rod outer portion 1052. The arms 1304 preferably have a lateral extension 1306 at the forward ends thereof. The arms 1304 are adapted to be operatively coupled with the protective cap 1014, when the protective cap 1014 is mounted over the forward end of the outer housing 1002.

Typically, two pairs of protrusions 1310 are formed, each one between two pairs of arms 1304. The two pairs of protrusions 1310 are disposed mutually opposite to each other and extend forwardly from the forward end 1300 of the plunger rod outer portion 1052. Each one of the protrusions 1310 has a tooth 1312, which includes a radially inwardly extending projection 1314 and a radially outwardly extending projection 1316.

It is seen in FIGS. 21A and 21B that several longitudinal ribs 1330 are formed on the outer surface of the plunger rod outer portion 1052 and are radially spaced from each other. The longitudinal ribs 1330 extend generally rearwardly from the forward end 1300 of the plunger rod outer portion 1052 and are adapted for engagement with the outer housing 1002. It is noted that a demarcation 1331 is provided generally between a pair of ribs 1330.

It is a particular feature of an embodiment of the present invention that two diametrically opposed ring engaging guiding tracks 1350 are formed on the plunger rod outer portion 1052 and communicates with the inner volume thereof. The ring engaging guiding track 1350 is generally disposed between longitudinal ribs 1330 and extends rearwardly from the forward end 1300 of the plunger rod outer portion 1052.

The ring engaging guiding track 1350 preferably includes a locking portion 1360, which is disposed adjacent the forward end 1300 of the plunger rod outer portion 1052 and extends transversely with respect to the longitudinal axis 1007. The locking portion 1360 has a first side wall 1362, a second side wall 1364 and a forwardly facing wall 1366, disposed generally transversely with respect to the first side wall 1362. The locking portion 1360 communicates with reconstitution portion 1370, which extends axially rearwardly from second side wall 1364 up to a priming portion 1380. The reconstitution portion 1370 extends in parallel to longitudinal axis 1007. The priming portion 1380 is preferably helical and extends at an angle relative to the longitudinal axis 1007 up to an injection portion 1390, which extends axially rearwardly from the priming portion 1380 up to a rearward most edge 1392. The injection portion 1390 has a side wall 1394 and it extends in parallel to longitudinal axis 1007. It is noted that the reconstitution portion 1370 and the injection portion 1390 are radially and axially offset from each other. Also, the locking portion 1360 and the priming portion are axially offset from each other. The helical portion 1380 is an at least partially helical groove that extends both radially and axially rearwardly from the reconstitution portion 1370.

It is specifically seen in FIG. 21C that a forwardly facing shoulder 1396 is formed on the inner surface of the plunger rod outer portion 1052, located generally adjacent the rearward end 1302 thereof, adapted for fixedly attaching the plunger rod inner portion 1054 with the plunger rod outer portion 1052.

It is also specifically seen in FIG. 21C that a plurality of protrusions 1398 are formed on the inner surface of the plunger rod outer portion 1052 and disposed adjacent the forward end 1300 thereof. The protrusions 1398 are radially spaced from each other and are adapted for operative engagement with the syringe holder 1020.

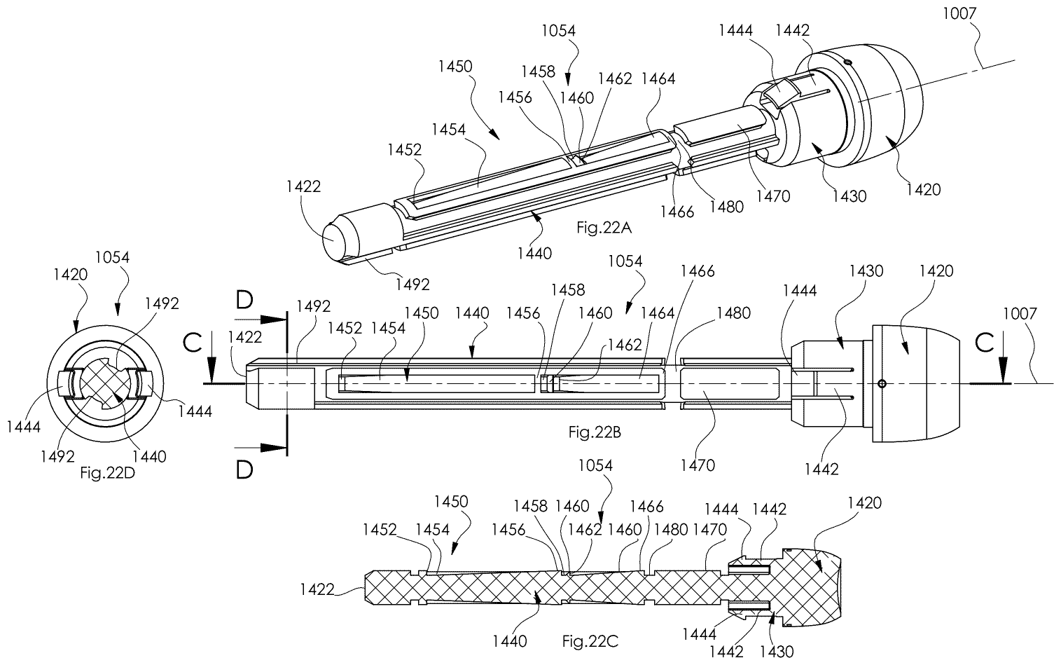

Reference is now made to FIGS. 22A-22D, which are respectively a simplified pictorial view, a planar side view and two simplified sectional views taken along lines C-C and D-D in FIG. 22B of the plunger rod inner portion 1054 forming part of the dual chamber injection system 1000 of FIGS. 14A & 14B.

In accordance with an embodiment of the present invention, the plunger rod inner portion 1054 is an integrally formed element, preferably injection molded of plastic and is arranged along longitudinal axis of symmetry 1007.

The plunger rod inner portion 1054 is a generally longitudinal cylindrical element having a bulkhead portion 1420 at a rearward end thereof and a piston engaging surface 1422 at a forward end thereof.

A connection portion 1430 is disposed between the bulkhead portion 1420 and a shaft portion 1440 of the plunger rod inner portion 1054, whereas the shaft portion 1440 terminates at the piston engaging surface 1422.

The connection portion 1430 generally includes two diametrically opposed snap portions 1442 extending forwardly from the bulkhead portion 1420 and having a radially outwardly extending tooth 1444 formed thereon for engagement with the plunger rod outer portion 1052.

It is seen in FIGS. 22A-22C that two diametrically opposed syringe holder engaging guiding tracks 1450 are formed on the shaft 1440 of the plunger rod inner portion 1054. The syringe holder engaging guiding tracks 1450 extend from a location adjacent the piston engaging surface 1422 to a location generally adjacent the connection portion 1430.

The syringe holder engaging guiding tracks 1450 include a rearwardly facing wall 1452, a rearwardly tapered reconstitution surface 1454 extending rearwardly from wall 1452 up to a step 1456, which continues to a recess 1458 formed between the step 1456 and a rearwardly tapered priming surface 1460. The rearwardly tapered priming surface 1460 terminates at a rearwardly facing wall 1462, and a rearwardly tapered injection surface 1464, which is disposed rearwardly of the wall 1462 and extends up to a rearward most edge 1466. A longitudinal protrusion 1470 is formed rearwardly of the rearward most edge 1466 and between the connection portion 1430 and the edge 1466, forming a locking gap 1480 between the rearward most edge 1466 and the longitudinal protrusion 1470.

It is specifically seen in FIG. 22D that typically two diametrically opposed longitudinal grooves 1492 are formed along the shaft portion 1440 of the plunger rod inner portion 1054 and extend axially rearwardly from the piston engaging surface 1422. The longitudinal grooves 1492 are adapted for operative engagement with the syringe holder 1020.

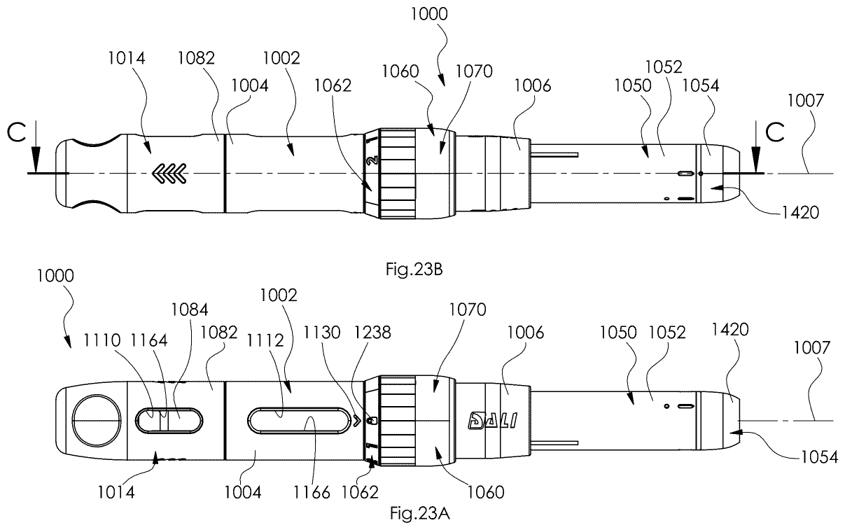

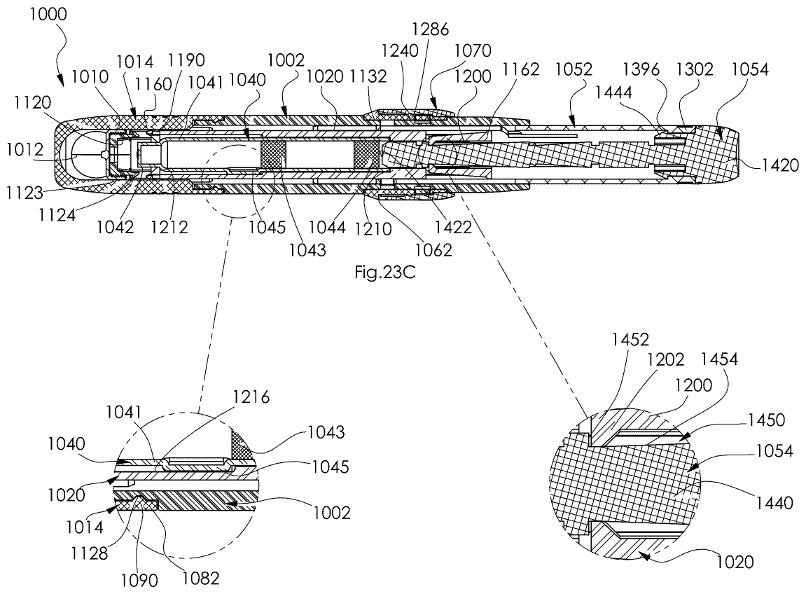

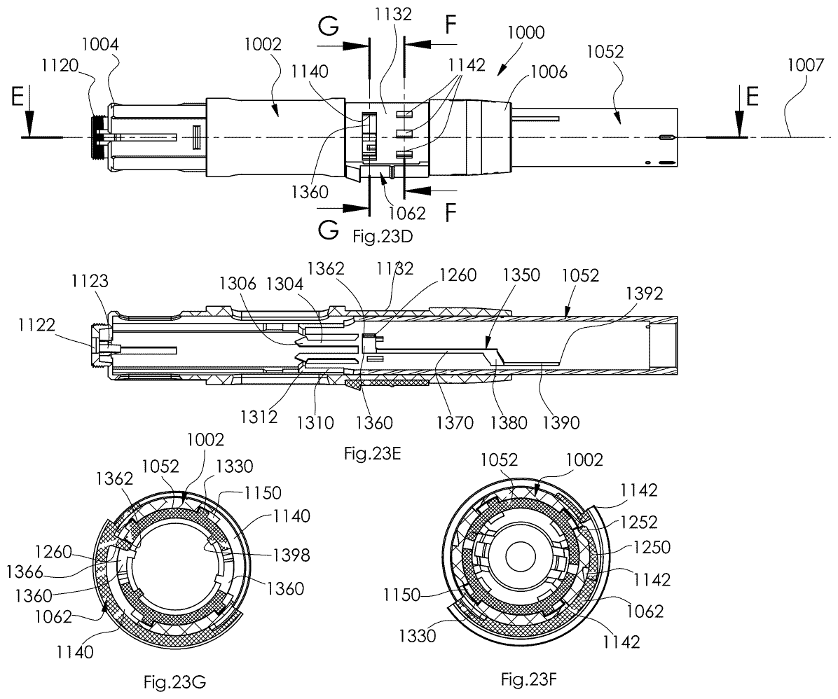

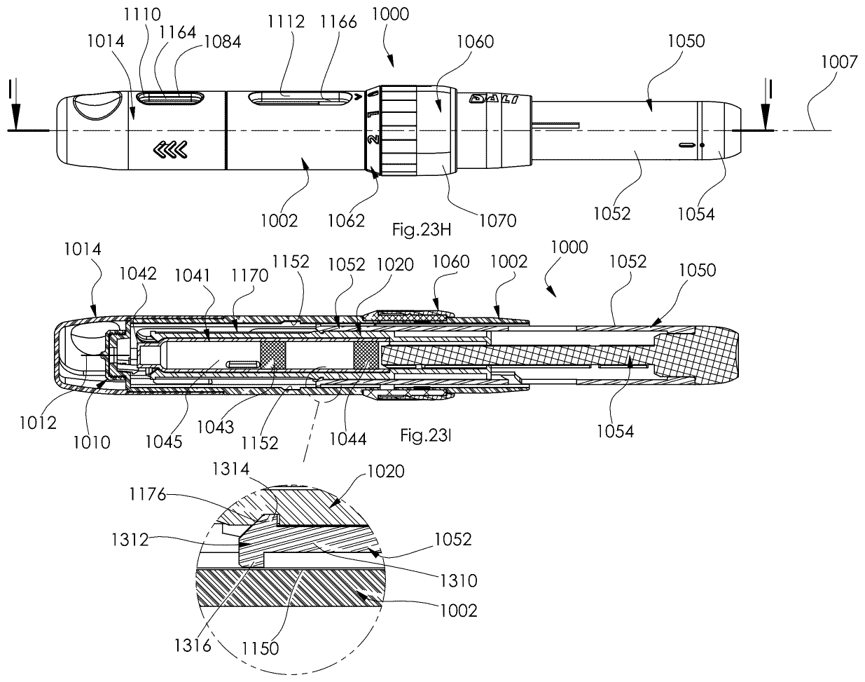

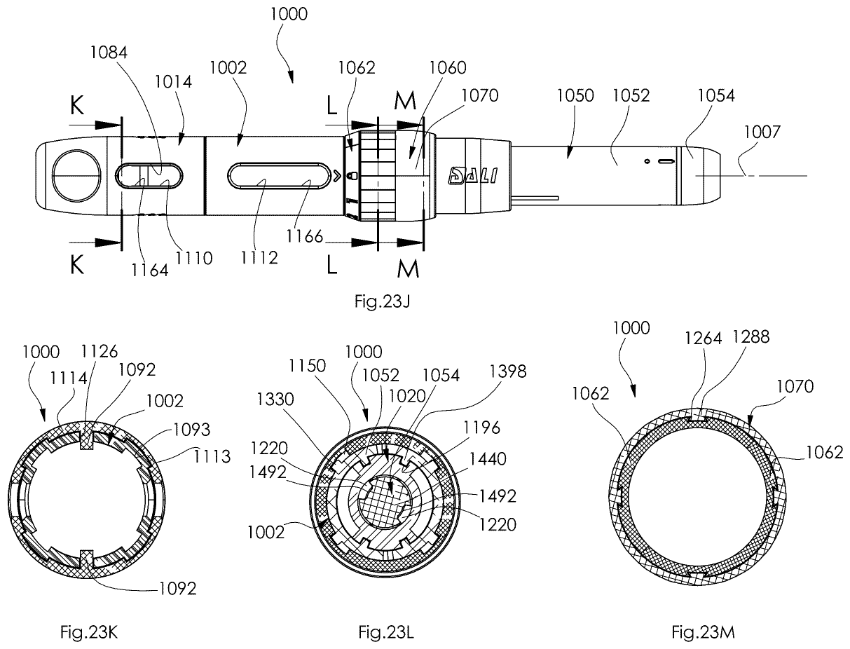

Reference is now made to FIGS. 23A-23C, which are simplified drawings of the dual chamber injection system 1000 of FIGS. 14A-22D in a locked operative orientation, including respectively two simplified different plan side views and a sectional view taken along lines C-C in FIG. 23B. Reference is additionally made to FIGS. 23D-23G, which are simplified drawings of the dual chamber injection system 1000 of FIGS. 14A-22D in a locked operative orientation, where only the outer housing 1002, the plunger rod outer portion 1052 and one ring portion 1062 is shown to clearly describe the interactions therebetween, including respectively a plan side view and three different sectional views taken along lines E-E, G-G and F-F in FIG. 23D.

Reference is also made to FIGS. 23H and 23I, which are simplified drawings of the dual chamber injection system 1000 of FIGS. 14A-22D in a locked operative orientation, including respectively a simplified plan side view and a sectional view taken along lines I-I in FIG. 23H. Reference is additionally made to FIGS. 23J-23M, which are simplified drawings of the dual chamber injection system 1000 of FIGS. 14A-22D in a locked operative orientation, including respectively a simplified plan side view and three sectional views taken along lines K-K, L-L and M-M in FIG. 23J, showing some of the components of the dual chamber injection system for clarity.

It is noted that only the outer housing 1002 and the protective cap 1014 are shown in FIG. 23K. The outer housing 1002, plunger rod outer portion 1052, syringe holder 1020 and plunger rod inner portion 1054 are shown in FIG. 23L and ring portions 1062 and collar 1070 are shown in FIG. 23M. The remaining components of the dual chamber injection system 1000 are hidden in these three sectional figures for clarity purposes.

The dual chamber injection system 1000 is seen in FIGS. 23A-23M in a locked operative orientation.

It is seen specifically in FIGS. 23C and 23I that cartridge 1040 is fixedly mounted into the syringe holder 1020 and is retained therein. The rearward end of the cartridge 1040 is supported against the forwardly facing shoulder 1210 of the syringe holder 1020 and the forward end of the cartridge 1040 is supported against curved forward surface 1212 of the syringe holder 1020. It is seen that the septum 1042 of the cartridge 1040 protrudes forwardly from the forward end 1160 of the syringe holder 1020 and the bypass protrusion 1045 of the cartridge 1040 is preferably guided along groove 1216 of the syringe holder 1020.

The forward portion of the syringe holder 1020 along with the cartridge 1040 that is housed therewithin are received within the outer housing 1002, such that in this locked operative orientation the forward end 1160 of the syringe holder 1020 is rearwardly spaced from the forward end 1004 of the outer housing 1002, particularly openings 1190 of the syringe holder 1020 are rearwardly spaced from outwardly extending teeth 1124 of snap portions 1123 of the outer housing 1002.

It is a particular feature of an embodiment of the present invention that the needle hub 1010 is threadably attached to externally threaded protrusion 1120 of the outer housing 1002, but the needle 1012 does not penetrate the septum 1042 of the cartridge 1040 in this locked operative orientation of the dual chamber injection system 1000.

It is particularly seen in FIG. 23C that the protective cap 1014 is mounted over the forward end 1004 of the outer housing 1002 and over the needle 1012, thereby protecting the needle 1012. Specifically, the protective cap 1014 is held on the outer housing 1002 due to engagement of protrusions 1090 of the protective cap 1014 with the recesses 1128 of the outer housing 1002.

It is also particularly seen in FIG. 23C that the rearward portion of the syringe holder 1020 is disposed within the plunger rod outer portion 1052, between the inner surface thereof and the shaft portion 1440 of the plunger rod inner portion 1054 and concentrically arranged with the plunger rod assembly 1050.

It is specifically seen in FIG. 23C that plunger rod inner portion 1054 is generally received into the plunger rod outer portion 1052, whereas the bulkhead portion 1420 of the plunger rod inner portion 1054 protrudes rearwardly from the plunger rod outer portion 1052. It is noted that the plunger rod inner portion 1054 is fixedly coupled to the plunger rod outer portion 1052 by means of engagement between forwardly facing shoulder 1396 of the plunger rod outer portion 1052 with radially outwardly extending teeth 1444 of the plunger rod inner portion 1054. The plunger rod inner portion 1054 is disposed concentrically with the plunger rod outer portion 1052. It is appreciated that alternatively the plunger rod outer portion 1052 and the plunger rod inner portion 1054 may be formed integrally as a single element.

It is further seen in FIGS. 23A-23C that the plunger rod assembly 1050 is slidably partially inserted into the outer housing 1002 and protrudes rearwardly therefrom.

It is seen that in this locked operative orientation the pistons 1043 and 1044 are rearwardly spaced relative to the bypass protrusion 1045. The rearward piston 1044 is rearwardly spaced from the forward piston 1043 and the piston engaging surface 1422 of the plunger rod inner portion 1054 is slightly rearwardly spaced from the rearward piston 1044. It is noted that drug preparation is confined between the forward piston 1043 and the septum 1042 of the medicament cartridge 1040 and a solvent is confined between the forward piston 1043 and the rearward piston 1044.

It is further seen in FIGS. 23A-23M that ring assembly 1060, including two generally identical ring portions 1062 that are fixedly attached to each other and collar 1070 encircling the ring portions 1062, is mounted over the recessed portion 1132 of the outer housing 1002.

It is a particular feature of an embodiment of the present invention that the ring assembly 1060 is rotatably disposed over the outer housing 1002.

It is particularly seen in FIG. 23C that the circumferential protrusion 1240 of the ring portions 1062 is inserted into the circumferential groove 1286 of the collar 1070, thereby the ring portions 1062 are attached to the collar 1070. It is additionally particularly seen in FIG. 23M that ribs 1288 of the collar 1070 are inserted into grooves 1264 of the ring portions 1062, thereby preventing rotation between the ring portions 1062 and the collar 1070, resulting in rotation of the ring assembly 1060 about the longitudinal axis 1007 as a single integral component.

It is a particular feature of an embodiment of the present invention, as particularly seen in FIG. 23C, that rearward axial displacement of the plunger assembly 1050 is prevented in this locked operative orientation due to engagement of snap portions 1200 of the syringe holder 1020 with the shaft portion 1440 of the plunger rod inner portion 1054. Specifically, radially inwardly directed teeth 1202 of snap portions 1200 of the syringe holder 1020 are supported against rearwardly facing wall 1452 of the plunger rod inner portion 1054, thereby preventing rearward displacement of the plunger rod inner portion 1054 relative to the syringe holder 1020 and relative to the outer housing 1002.

It is a particular feature of an embodiment of the present invention that forward axial displacement of the plunger assembly 1050 relative to the syringe holder 1020 is guided by means of engagement between snap portions 1200 of the syringe holder 1020 and syringe holder engaging guiding tracks 1450 of the plunger rod inner portion 1054.

It is particularly seen in FIG. 23A that a visual indication is provided to the user about transitions between different operative orientations during the process of medicament reconstitution, priming and injection of the medicament from the medicament cartridge 1040.

Specifically, in this locked operative orientation, it is seen in FIG. 23A that the demarcation 1130 on the outer housing 1002 is aligned with one of the demarcations 1238 on the ring portion 1062. In this locked operative orientation, the demarcation 1130 is preferably aligned with a lock sign.

It is additionally noted that the user can inspect the medicament through window 1112 of the outer housing 1002, which is aligned with window 1166 of the syringe holder 1020, as well as through window 1084 of the protective cap 1014, which is aligned with window 1110 of the outer housing 1002 and window 1164 of the syringe holder 1020.

Reference is now specifically made to FIGS. 23D-23G, which only illustrate interactions between one ring portion 1062, plunger rod outer portion 1052 and the outer housing 1002 to clarify the different engagements between these components of the dual chamber injection system 1000.

It is a particular feature of an embodiment of the present invention, as particularly seen in FIGS. 23E and 23G, that the forward axial displacement of the plunger assembly 1050 relative to the syringe holder 1020 and relative to the outer housing 1002 is prevented by means of engagement of the ring portions 1062 with the ring engaging guiding tracks 1350 of the plunger rod outer portion 1052. Specifically, in this locked operative orientation of the dual chamber injection system 1000, the radially inwardly extending teeth 1260 of the ring portion 1062 are supported against the forwardly facing wall 1366 of the locking portions 1360 of the ring engaging guiding tracks 1350 of the plunger rod outer portion 1052, thereby preventing axial displacement of the plunger assembly 1050.

It is noted that the radially inwardly extending teeth 1260 preferably extend into the locking portions 1360 of the ring engaging guiding tracks 1350 through openings 1140 formed in the outer housing 1002.

It is a particular feature of an embodiment of the present invention that the ring portions 1062 are disposed at a certain angular orientation relative to the outer housing 1002 in this locked operative orientation of the dual chamber injection system 1000 and the ring assembly 1060 is adapted to rotate about longitudinal axis 1007 relative to the outer housing 1002.

It is specifically seen in FIG. 23F that radially inwardly extending fingers 1252 of snap portions 1250 of the ring portion 1062 are seated within one of the recesses 1142 of the outer housing 1002 in this locked operative orientation. It is a particular feature of an embodiment of the present invention that this engagement between radially inwardly extending fingers 1252 and recesses 1142 acts as a ratchet and prevents rotation of the ring portions 1062 relative to the outer housing 1002 in one rotational direction, but allows rotation thereof in an opposite rotational direction.

It is noted that the plunger rod outer portion 1052 is prevented from rotation relative to the outer housing 1002 due to engagement of longitudinal ribs 1330 of the plunger rod outer portion 1052 with grooves 1150 of the outer housing 1002.

It is a particular feature of an embodiment of the present invention, as particularly seen in FIG. 23I, that relative axial displacement between the syringe holder 1020 and the plunger assembly 1050 is prevented due to engagement of the plunger rod outer portion 1052 both with the syringe holder 1020 and with the outer housing 1002. It is specifically seen in FIG. 23I that radially inwardly extending projections 1314 of teeth 1312 of protrusions 1310 of the plunger rod outer portion 1052 are seated within recesses 1176 of the syringe holder 1020. The radially outwardly extending projections 1316 of protrusions 1310 are supported against the grooves 1150 formed on the inner surface of the outer housing 1002 and thereby are prevented from being radially outwardly deflected and disengaged from recesses 1176 of the syringe holder 1020.

It is noted that in this locked operative orientation of the dual chamber injection system 1000, the protrusions 1310 of the plunger rod outer portion 1052 are rearwardly spaced from recesses 1152 formed within grooves 1150 on the inner surface of the outer housing 1002.

It is specifically seen in FIG. 23K that protective cap 1014 is non-rotatably coupled to the outer housing 1002. Rotation therebetween is prevented due to engagement of protrusions 1113 of the outer housing 1002 with recessed surfaces 1093 of the protective cap 1014.

It is additionally seen in FIG. 23K that ribs 1092 of the protective cap 1014 are inserted into grooves 1126 of the outer housing 1002 and as long as the protective cap 1014 is mounted onto the outer housing 1002, forward axial displacement of the plunger assembly 1050 beyond a certain point at the end of priming operative orientation is prevented, as is described in detail hereinbelow.

It is particularly seen in FIG. 23L that the plunger rod outer portion 1052 is prevented from rotation relative to the outer housing 1002 due to engagement of longitudinal ribs 1330 of the plunger rod outer portion 1052 with grooves 1150 of the outer housing 1002.

It is additionally seen in FIG. 23L that rotation is prevented between the syringe holder 1020 and the plunger rod outer portion 1052 by means of engagement of protrusions 1398 of the plunger rod outer portion 1052 in grooves 1196 of the syringe holder 1020.

It is also seen in FIG. 23L that rotation is prevented between syringe holder 1020 and the plunger rod inner portion 1054 by means of engagement of protrusions 1220 of the syringe holder 1020 within grooves 1492 of the plunger rod inner portion 1054.

It is thus a particular feature of an embodiment of the present invention that relative rotation is prevented between the outer housing 1002, the syringe holder 1020, the plunger rod outer portion 1052 and the plunger rod inner portion 1054 in all operative orientations of the dual chamber injection system 1000.

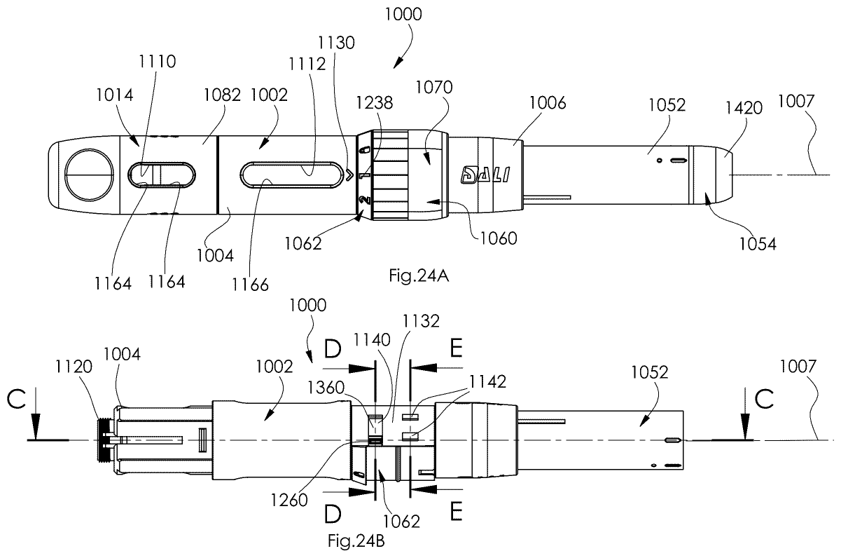

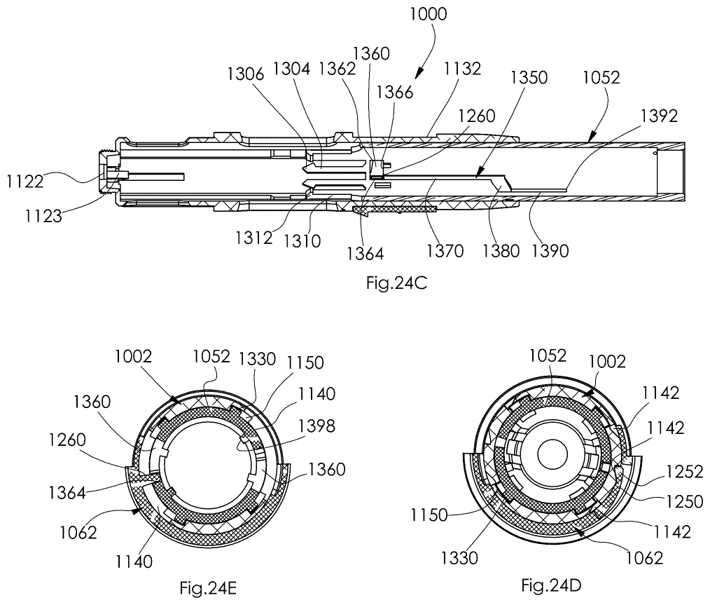

Reference is now made to FIGS. 24A-24E, which are simplified drawings of the dual chamber injection system 1000 of FIGS. 14A-22D in an unlocked operative orientation, including respectively two simplified different plan side views and three sectional views taken respectively along lines C-C in FIG. 24B and lines D-D and E-E in FIG. 24A.

The dual chamber injection system 1000 is seen in FIGS. 24A-24E in an unlocked operative orientation, before initiation of medicament reconstitution. It is seen particularly in FIG. 24A that the plunger rod assembly 1050 is disposed in the same axial orientation relative to the outer housing 1002 as shown in FIGS. 23A-23M. It is also particularly seen in FIGS. 24A-24E that the ring assembly 1060 is rotated about longitudinal axis 1007, generally in a clockwise direction, relative to the outer housing 1002, as compared to the locked orientation of the dual chamber injection system 1000 shown in FIGS. 24A-24E.

It is seen that in this unlocked operative orientation that the pistons 1043 and 1044 remain rearwardly spaced relative to the bypass protrusion 1045. The rearward piston 1044 is rearwardly spaced from the forward piston 1043 and the plunger assembly 1050 is not displaced axially forwardly relative to the outer housing 1002 or relative to the syringe holder 1020 in this unlocked operative orientation as compared to the locked operative orientation illustrated in FIGS. 23A-23M.

The forward portion of the syringe holder 1020 along with the cartridge 1040 that is housed therewithin are received within the outer housing 1002, such that in this unlocked operative orientation the forward end 1160 of the syringe holder 1020 remains rearwardly spaced from the forward end 1004 of the outer housing 1002, particularly openings 1190 of the syringe holder 1020 remain rearwardly spaced from outwardly extending teeth 1124 of snap portions 1123 of the outer housing 1002.

It is noted that the needle hub 1010 is threadably attached to externally threaded protrusion 1120 of the outer housing 1002, but the needle 1012 does not penetrate the septum 1042 of the cartridge 1040 in this unlocked operative orientation of the dual chamber injection system 1000.

It is a particular feature of an embodiment of the present invention that forward axial displacement of the plunger assembly 1050 relative to the syringe holder 1020 is guided by means of engagement between snap portions 1200 of the syringe holder 1020 and syringe holder engaging guiding tracks 1450 of the plunger rod inner portion 1054.

Since there is no relative axial displacement between the syringe holder 1020 and the plunger assembly 1050 in this unlocked operative orientation, snap portions 1200 of the syringe holder 1020 remain in the same axial position relative to the shaft portion 1440 of the plunger rod inner portion 1054, such as illustrated in FIG. 23C. Specifically, radially inwardly directed teeth 1202 of snap portions 1200 of the syringe holder 1020 are supported against rearwardly facing wall 1452 of the plunger rod inner portion 1054.

It is particularly seen in FIG. 24A that the protective cap 1014 remains mounted over the forward end 1004 of the outer housing 1002 and over the needle 1012, thereby protecting the needle 1012. The ribs 1092 of the protective cap 1014 remain inserted into grooves 1126 of the outer housing 1002 and as long as the protective cap 1014 is mounted onto the outer housing 1002, forward axial displacement of the plunger assembly 1050 beyond a certain point at the end of priming operative orientation is prevented, as is described in detail hereinbelow. Additionally, protrusions 1113 of the protective cap 1014 remain supported within recessed surfaces 1093 of the outer housing 1002.

It is noted that most of the spatial relationships between the different components of the dual chamber injection system 1000 remain the same as described and illustrated with respect to FIGS. 23A-23M, besides the following:

It is a particular feature of an embodiment of the present invention that in this unlocked operative orientation, the ring assembly 1060 is rotated in a first rotational direction, preferably clockwise direction about longitudinal axis 1007, thereby positioning the dual chamber injection system 1000 in the unlocked operative orientation. In this unlocked operative orientation, the demarcation 1130 on the outer housing 1002 is aligned with another demarcation 1238 on the ring portion 1062, as compared to FIG. 23A, preferably a demarcation entitled as “1”.

Reference is now specifically made to FIGS. 24B-24E, which only illustrate interactions between one ring portion 1062, plunger rod outer portion 1052 and the outer housing 1002 to clarify the different engagements between these components of the dual chamber injection system 1000.

It is a particular feature of an embodiment of the present invention, as particularly seen in FIGS. 24C and 24E, that the forward axial displacement of the plunger assembly 1050 relative to the outer housing 1002 is permitted in this unlocked operative orientation. Specifically, in this unlocked operative orientation of the dual chamber injection system 1000, following rotation of the ring assembly 1060, the radially inwardly extending teeth 1260 of the ring portion 1062 are supported against the second side walls 1364 of the locking portions 1360 of the ring engaging guiding tracks 1350 of the plunger rod outer portion 1052, thereby permitting axial displacement of the plunger assembly 1050.

It is noted that the radially inwardly extending teeth 1260 preferably extend into the locking portions 1360 of the ring engaging guiding tracks 1350 through openings 1140 formed in the outer housing 1002 and are now disengaged from the forwardly facing wall 1366 of the locking portions 1360 and is disposed in alignment with the reconstitution portion 1370 of the ring engaging guiding tracks 1350 formed on the plunger rod outer portion 1052.

It is specifically seen in FIG. 24D that following rotation of the ring assembly 1060 relative to the outer housing 1002, the radially inwardly extending fingers 1252 of snap portions 1250 of the ring portion 1062 are seated within another one of the recesses 1142 of the outer housing 1002 in this unlocked operative orientation, as compared to their position as shown in FIG. 23F. It is a particular feature of an embodiment of the present invention that this engagement between radially inwardly extending fingers 1252 and recesses 1142 prevents rotation of the ring assembly 1060 relative to the outer housing 1002 in an opposite rotational direction, preferably in a counter-clockwise direction.

It is noted that relative axial displacement between the syringe holder 1020 and the plunger assembly 1050 is prevented due to engagement of the plunger rod outer portion 1052 both with the syringe holder 1020 and with the outer housing 1002. Specifically, the radially inwardly extending projections 1314 of teeth 1312 of protrusions 1310 of the plunger rod outer portion 1052 remain seated within recesses 1176 of the syringe holder 1020. The radially outwardly extending projections 1316 of protrusions 1310 are supported against the grooves 1150 formed on the inner surface of the outer housing 1002 and thereby are prevented from being radially outwardly deflected and disengaged from recesses 1176 of the syringe holder 1020.

It is noted that in this unlocked operative orientation of the dual chamber injection system 1000, the protrusions 1310 of the plunger rod outer portion 1052 remain rearwardly spaced from recesses 1152 formed on the inner surface of the outer housing 1002.

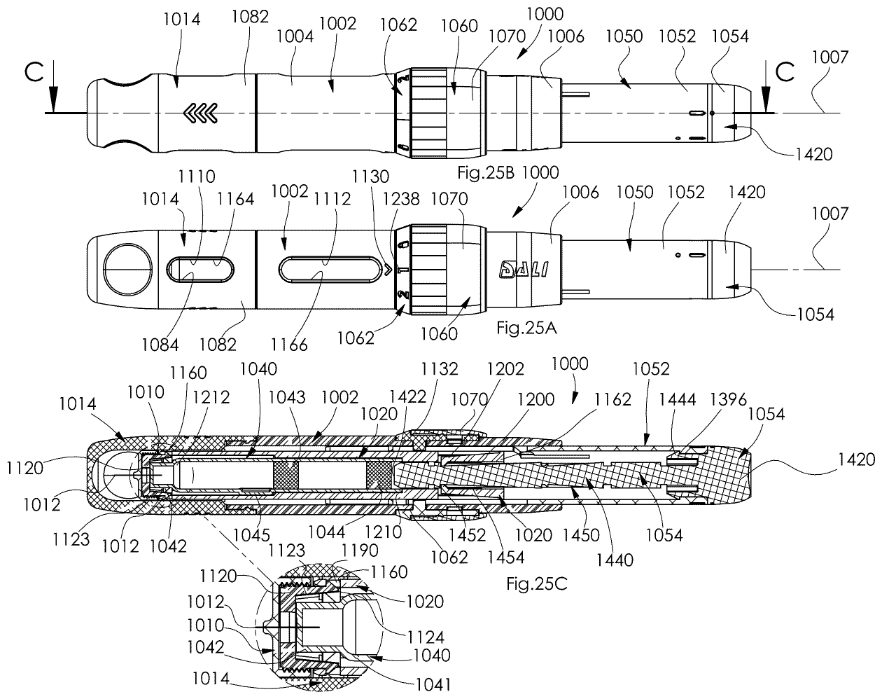

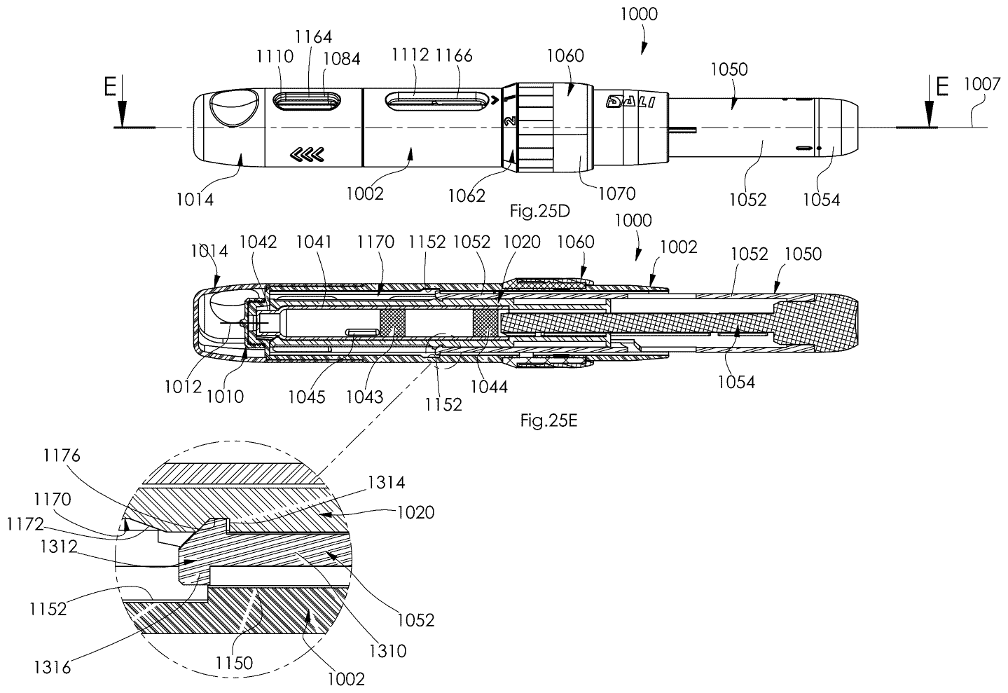

Reference is now made to FIGS. 25A-25C, which are simplified drawings of the dual chamber injection system 1000 of FIGS. 14A-22D in a septum penetration operative orientation, including respectively two simplified different plan side views and a sectional view taken along lines C-C in FIG. 25B. Reference is additionally made to FIGS. 25D and 25E, which are simplified drawings of the dual chamber injection system 1000 of FIGS. 14A-22D in a septum penetration operative orientation, including respectively a simplified plan side view and a sectional view taken along lines E-E in FIG. 25D.

It is seen that in this septum penetration operative orientation that the pistons 1043 and 1044 remain rearwardly spaced relative to the bypass protrusion 1045. The rearward piston 1044 is rearwardly spaced from the forward piston 1043 and the plunger assembly 1050 along with the syringe holder 1020 are displaced axially forwardly relative to the outer housing 1002 in this septum penetration operative orientation as compared to the unlocked operative orientation illustrated in FIGS. 24A-24E.

It is a particular feature of an embodiment of the present invention that forward axial displacement of the plunger assembly 1050 relative to the syringe holder 1020 is guided by means of engagement between inwardly extending teeth 1260 of the ring portions 1062 and the ring engaging guiding tracks 1350 of the plunger rod outer portion 1052 in all operative orientations of the dual chamber injection system 1000.

It is a further particular feature of an embodiment of the present invention that forward axial displacement of the plunger assembly 1050 relative to the syringe holder 1020 is guided by means of engagement between snap portions 1200 of the syringe holder 1020 and syringe holder engaging guiding tracks 1450 of the plunger rod inner portion 1054 in order to prevent rearward axial displacement of the plunger assembly 1050 relative to the syringe holder 1020 in all operative orientations of the dual chamber injection system 1000.

Since there is no relative axial displacement between the syringe holder 1020 and the plunger assembly 1050 in this septum penetration operative orientation, snap portions 1200 of the syringe holder 1020 remain in the same axial position relative to the shaft portion 1440 of the plunger rod inner portion 1054, such as illustrated in FIG. 23C. Specifically, radially inwardly directed teeth 1202 of snap portions 1200 of the syringe holder 1020 are supported against rearwardly facing wall 1452 of the plunger rod inner portion 1054.

It is particularly seen in FIGS. 25A-25E that the protective cap 1014 remains mounted over the forward end 1004 of the outer housing 1002 and over the needle 1012, thereby protecting the needle 1012. The ribs 1092 of the protective cap 1014 remain inserted into grooves 1126 of the outer housing 1002 and as long as the protective cap 1014 is mounted onto the outer housing 1002, forward axial displacement of the plunger assembly 1050 beyond a certain point at the end of priming operative orientation is prevented, as is described in detail hereinbelow. Additionally, protrusions 1113 of the protective cap 1014 remain supported within recessed surfaces 1093 of the outer housing 1002.

It is noted that most of the spatial relationships between the different components of the dual chamber injection system 1000 remain the same as described and illustrated with respect to FIGS. 24A-24E, besides the following:

It is a particular feature of an embodiment of the present invention that the plunger assembly 1050 along with the syringe holder 1020 are axially displaced forwardly along longitudinal axis 1007, such that in this septum penetration operative orientation the forward end 1160 of the syringe holder 1020 now abuts the forward end 1004 of the outer housing 1002, particularly outwardly extending teeth 1124 of snap portions 1123 of the outer housing 1002 are now engaged with openings 1190 of the syringe holder 1020.

It is noted that the needle 1012 of the needle hub 1010 now penetrates the septum 1042 of the cartridge 1040 in this septum penetration operative orientation of the dual chamber injection system 1000, thus fluid communication is established between the needle 1012 and the contents of the cartridge 1040.

It is another particular feature of an embodiment of the present invention that no further displacement between the syringe housing 1020 and the outer housing 1002 is permitted after engagement of the outwardly extending teeth 1124 of snap portions 1123 of the outer housing 1002 with openings 1190 of the syringe holder 1020.

It is noted that in this septum penetration operative orientation, the ring assembly 1060 remains in the same rotational orientation relative to the outer housing 1002 as illustrated in FIGS. 24A-24E.

It is noted that in this septum penetration operative orientation of the dual chamber injection system 1000, the forward axial displacement of the plunger assembly 1050 relative to the outer housing 1002 is permitted. Specifically, in this septum penetration operative orientation of the dual chamber injection system 1000, the radially inwardly extending teeth 1260 of the ring portion 1062 remain supported against the second side walls 1364 of the locking portions 1360 of the ring engaging guiding tracks 1350 of the plunger rod outer portion 1052, thereby permitting axial displacement of the plunger assembly 1050 relative to the outer housing 1002.

It is noted that relative axial displacement between the syringe holder 1020 and the plunger assembly 1050 is still prevented in this septum penetration operative orientation due to engagement of the plunger rod outer portion 1052 both with the syringe holder 1020 and with the outer housing 1002. Specifically, the radially inwardly extending projections 1314 of teeth 1312 of protrusions 1310 of the plunger rod outer portion 1052 remain seated within recesses 1176 of the syringe holder 1020, as particularly seen in FIG. 25E. The radially outwardly extending projections 1316 of protrusions 1310 are still supported against the inner surface of the outer housing 1002 and thereby are prevented from being radially outwardly deflected and disengaged from recesses 1176 of the syringe holder 1020.

It is a particular feature of an embodiment of the present invention that in this septum penetration operative orientation of the dual chamber injection system 1000 following axial forward displacement of the syringe holder 1020 and the plunger assembly 1050 relative to the outer housing 1002, the protrusions 1310 of the plunger rod outer portion 1052 are preferably less rearwardly spaced from recesses 1152 formed on the inner surface of the outer housing 1002 and are now disposed in vicinity of recesses 1152.

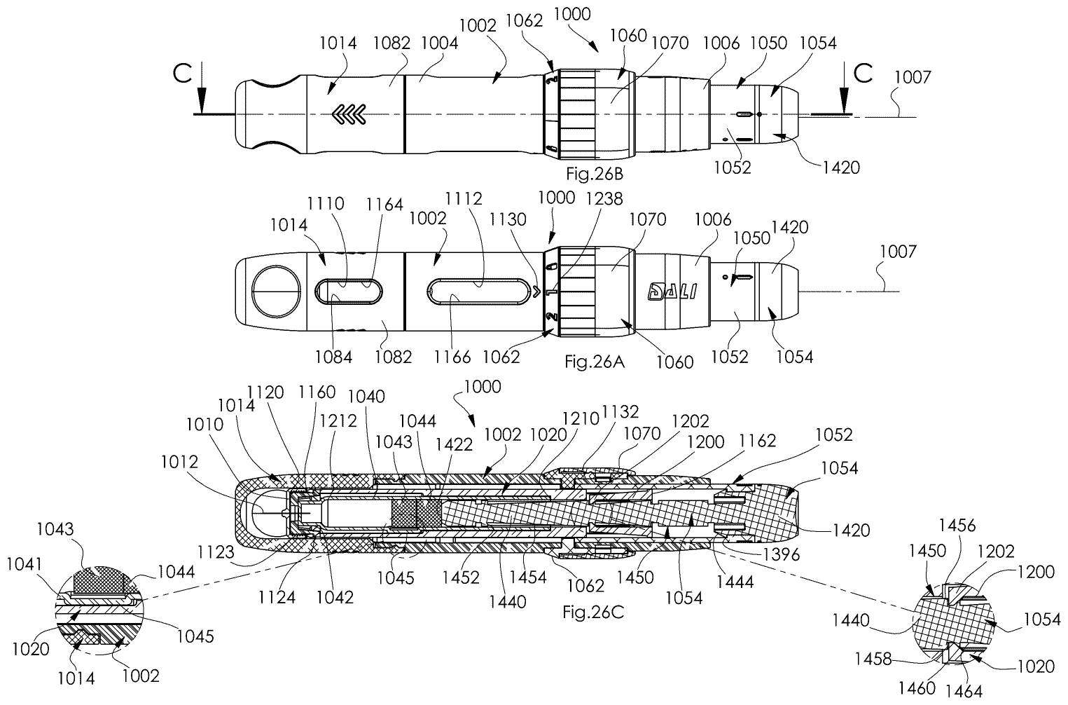

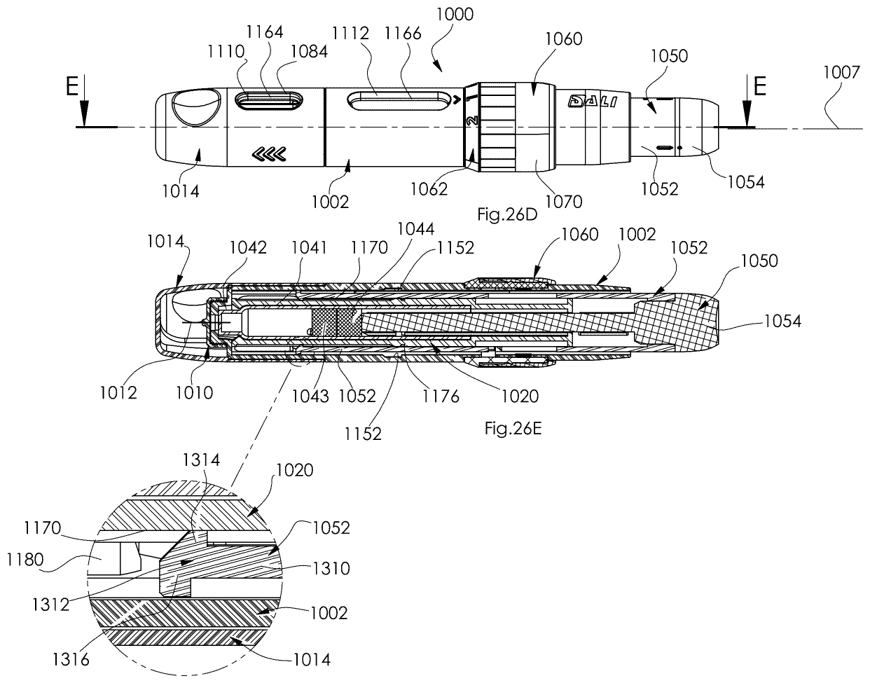

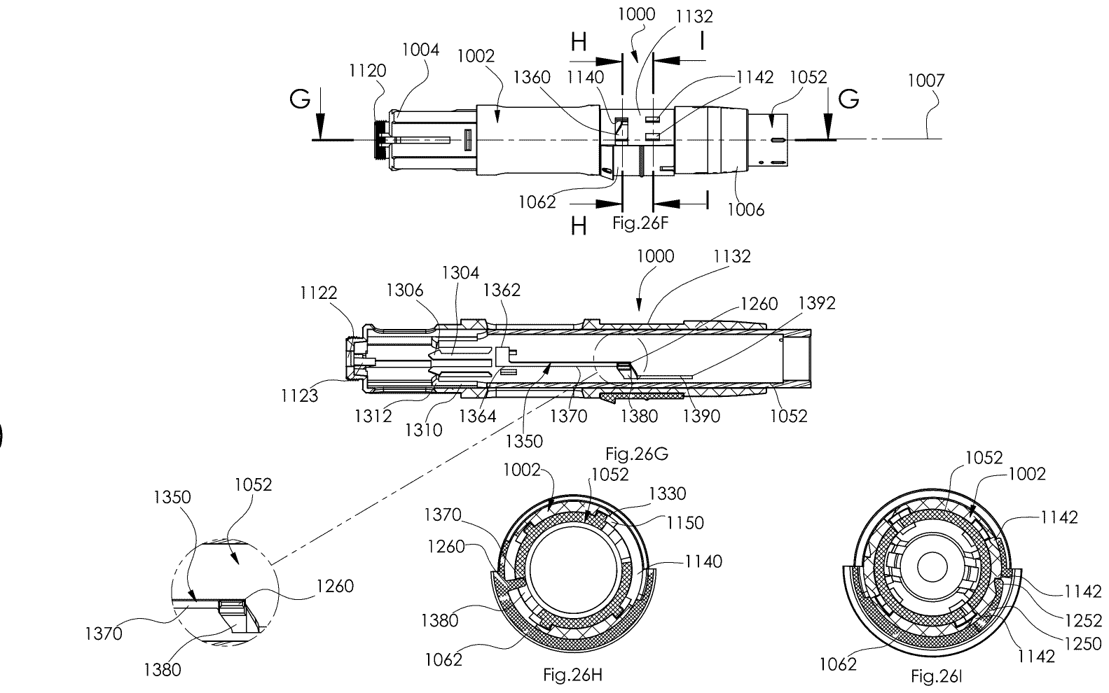

Reference is now made to FIGS. 26A-26C, which are simplified drawings of the dual chamber injection system 1000 of FIGS. 14A-22D in a reconstitution operative orientation, including respectively two simplified different plan side views and a sectional view taken along lines C-C in FIG. 26B. Reference is additionally made to FIGS. 26D and 26E, which are simplified drawings of the dual chamber injection system 1000 of FIGS. 14A-22D in the reconstitution operative orientation, including respectively a simplified plan side view and a sectional view taken along lines E-E in FIG. 26D. Reference is further made to FIGS. 26F-26I, which are simplified drawings of the dual chamber injection system 1000 of FIGS. 14A-22D in the reconstitution operative orientation, where only the outer housing 1002, the plunger rod outer portion 1052 and one ring portion 1062 is shown to clearly describe the interactions therebetween, including respectively a plan side view and three different sectional views taken along lines G-G, H-H and I-I in FIG. 26F.

It is seen that in this reconstitution operative orientation that the solvent was transferred from between the two pistons 1043 and 1044 into the chamber formed between the forward piston 1043 and the septum 1042 through the bypass protrusion 1045. The pistons 1043 and 1044 now abut each other and the forward piston 1043 is now disposed in front of the bypass protrusion 1045. The plunger assembly 1050 is now axially displaced both relative to the syringe holder 1020 and relative to the outer housing 1002 in this reconstitution operative orientation as compared to the septum penetration operative orientation illustrated in FIGS. 25A-25E.

It is particularly seen in FIGS. 26A-26E that the protective cap 1014 remains mounted over the forward end 1004 of the outer housing 1002 and over the needle 1012, thereby protecting the needle 1012. The ribs 1092 of the protective cap 1014 remain inserted into grooves 1126 of the outer housing 1002 and as long as the protective cap 1014 is mounted onto the outer housing 1002, forward axial displacement of the plunger assembly 1050 beyond a certain point at the end of priming operative orientation is prevented, as is described in detail hereinbelow. Additionally, protrusions 1113 of the protective cap 1014 remain supported within recessed surfaces 1093 of the outer housing 1002.

It is noted that most of the spatial relationships between the different components of the dual chamber injection system 1000 remain the same as described and illustrated with respect to FIGS. 25A-25E, besides the following:

It is a particular feature of an embodiment of the present invention that the plunger assembly 1050 in this reconstitution operative orientation is further axially displaced forwardly along longitudinal axis 1007, now both relative to the syringe holder 1020 and relative to the outer housing 1002.

It is noted that in this reconstitution operative orientation, the ring assembly 1060 remains in the same rotational orientation relative to the outer housing 1002 as illustrated in FIGS. 25A-25E. Thus, the orientation of the ring assembly 1060 relative to outer housing 1002 as illustrated in FIGS. 24D & 24E remains preferably identical to the orientation of the ring assembly 1060 as illustrated in FIGS. 26H & 26I.

It is noted that in this reconstitution operative orientation of the dual chamber injection system 1000, the forward axial displacement of the plunger assembly 1050 relative to the outer housing 1002 is permitted. Specifically, in this reconstitution operative orientation of the dual chamber injection system 1000, the radially inwardly extending teeth 1260 of the ring portion 1062 are guided along the reconstitution portion 1370 of the ring engaging guiding tracks 1350 up to engagement of the inwardly extending teeth 1260 with the priming portion 1380 of the ring engaging guiding tracks 1350, whereas further axial displacement of the plunger assembly 1050 is prevented by abutment of the teeth 1260 with a wall of the priming portion 1380 and resulting in an end of reconstitution position.

It is specifically seen in FIG. 26E that relative axial displacement between the syringe holder 1020 and the plunger assembly 1050 is now permitted in this reconstitution operative orientation due to disengagement of the plunger rod outer portion 1052 from the syringe holder 1020. Specifically, it is noted that along the forward axial displacement of the plunger assembly 1050 along with the syringe holder 1020 relative to the outer housing 1002, radially outwardly extending projections 1316 of the protrusions 1310 of the plunger rod outer portion 1052 become located in front of recesses 1152 formed on the inner surface of the outer housing 1002, thereby allowing the protrusions 1310 to deflect outwardly, resulting in release of the radially inwardly extending projections 1314 of teeth 1312 of protrusions 1310 of the plunger rod outer portion 1052 from recesses 1176 of the syringe holder 1020.

It is noted, as seen in FIG. 26E, that following forward axial displacement of the plunger assembly 1050, the protrusions 1310 of the plunger rod outer portion 1052 are now forwardly spaced from recesses 1152 formed within grooves 1150 on the inner surface of the outer housing 1002 and from recess 1176 of the syringe holder 1020. It is seen specifically in FIG. 26E that the protrusions 1310 return to their non-deformed orientation once they are disposed forwardly of recesses 1152.

It is a particular feature of an embodiment of the present invention that forward axial displacement of the plunger assembly 1050 relative to the syringe holder 1020 is guided by means of engagement between snap portions 1200 of the syringe holder 1020 and syringe holder engaging guiding tracks 1450 of the plunger rod inner portion 1054.

Following relative axial displacement of the plunger assembly 1050 relative to the syringe holder 1020 in this reconstitution operative orientation, as particularly seen in FIG. 26C, snap portions 1200 of the syringe holder 1020 are now more rearwardly disposed relative to the shaft portion 1440 of the plunger rod inner portion 1054, as compared to the illustrated in FIG. 23C. Specifically, radially inwardly directed teeth 1202 of snap portions 1200 of the syringe holder 1020 are now seated within recess 1458 of syringe holder engaging guiding tracks 1450 of the plunger rod inner portion 1054.

Displacement of the plunger assembly 1050 rearwardly is prevented in this reconstitution operative orientation due to engagement of the radially inwardly directed teeth 1202 with step 1456 of syringe holder engaging guiding tracks 1450 of the plunger rod inner portion 1054.

It is specifically seen in FIG. 26C that the solvent was transferred from between the two pistons 1043 and 1044 into the chamber formed between the forward piston 1043 and the septum 1042 through the bypass protrusion 1045, thus the reconstituted medicament is now disposed between the forward piston 1043 and the septum 1042. The pistons 1043 and 1044 now abut each other and the forward piston 1043 is now disposed in front of the bypass protrusion 1045.

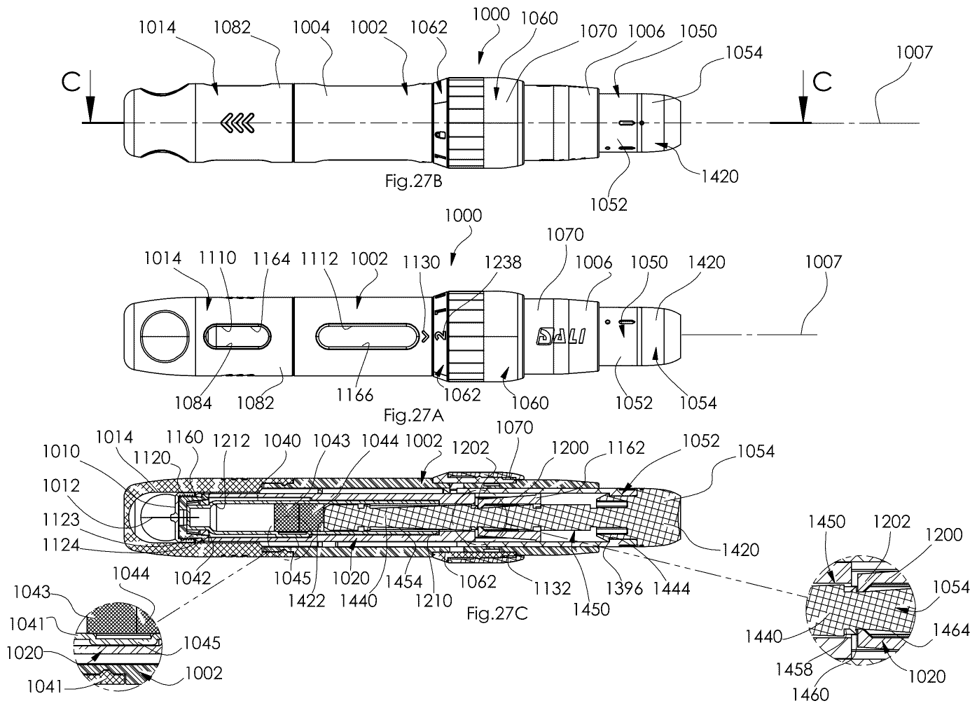

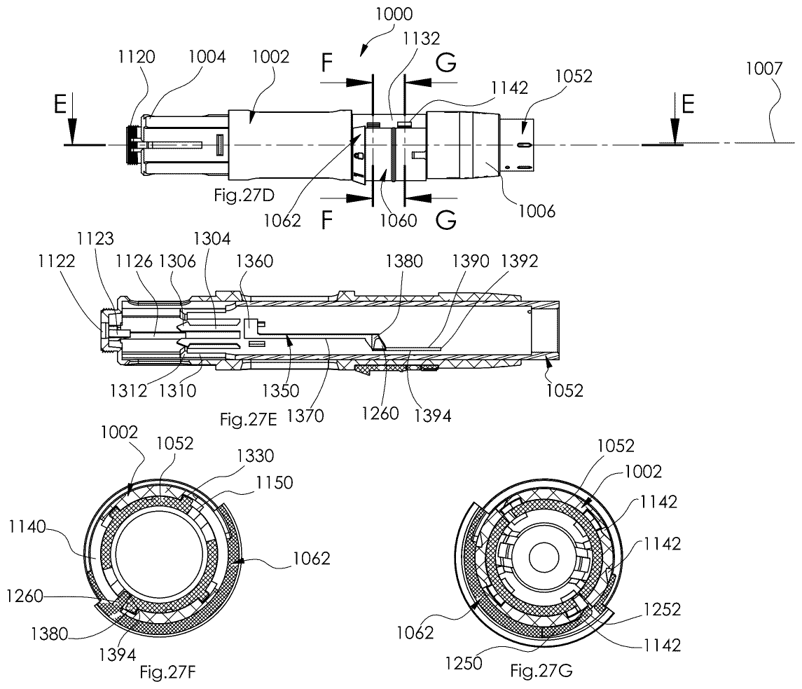

Reference is now made to FIGS. 27A-27C, which are simplified drawings of the dual chamber injection system 1000 of FIGS. 14A-22D in a priming operative orientation, including respectively two simplified different plan side views and a sectional view taken along lines C-C in FIG. 27B. Reference is additionally made to FIGS. 27D-27G, which are simplified drawings of the dual chamber injection system 1000 of FIGS. 14A-22D in a priming operative orientation, where only the outer housing 1002, the plunger rod outer portion 1052 and one ring portion 1062 is shown to clearly describe the interactions therebetween, including respectively a plan side view and three different sectional views taken along lines E-E, F-F and G-G in FIG. 27D.

It is seen that in this priming operative orientation the pistons 1043 and 1044 are disposed partially forwardly of the bypass protrusion 1045. The plunger assembly 1050 is urged to be displaced axially relative to the syringe holder 1020 and relative to the outer housing 1002 in this priming operative orientation as compared to the position of the plunger assembly 1050 at the end of reconstitution position, as specifically illustrated in FIGS. 26G-26I.

It is noted that at the end of this priming operative orientation and prior to injection operative orientation, air may be removed from the chamber formed between the forward piston 1043 and the septum 1042 along with a small amount of reconstituted medicament that may be ejected from the medicament cartridge 1040 through the needle 1012.

It is particularly seen in FIGS. 27A-27C that the protective cap 1014 remains mounted over the forward end 1004 of the outer housing 1002 and over the needle 1012, thereby protecting the needle 1012. The ribs 1092 of the protective cap 1014 remain inserted into grooves 1126 of the outer housing 1002 and as long as the protective cap 1014 is mounted onto the outer housing 1002, forward axial displacement of the plunger assembly 1050 beyond a certain point at the end of priming operative orientation is prevented, as is described in detail hereinbelow. Additionally, protrusions 1113 of the protective cap 1014 remain supported within recessed surfaces 1093 of the outer housing 1002.

It is noted that most of the spatial relationships between the different components of the dual chamber injection system 1000 remain the same as described and illustrated with respect to FIGS. 26A-26I, besides the following:

It is a particular feature of an embodiment of the present invention that in this priming operative orientation, the ring assembly 1060 is again rotated in a first rotational direction, preferably clockwise direction about longitudinal axis 1007, thereby positioning the dual chamber injection system 1000 in the priming operative orientation. In this priming operative orientation, the demarcation 1130 on the outer housing 1002 is aligned with another demarcation 1238 on the ring portion 1062, as compared to FIG. 24A, preferably a demarcation entitled as “2”.

It is a particular feature of an embodiment of the present invention that the plunger assembly 1050 in this priming operative orientation is caused to be displaced axially forwardly due to thread-like engagement of the radially inwardly extending tooth 1260 of the ring portion 1062 with the helical priming portion 1380 of the ring engaging guiding track 1350 of the plunger rod outer portion 1052. Specifically, as particularly seen in FIG. 27E, when the ring assembly 1060 is rotated, the inwardly extending tooth 1260 is guided along the priming portion 1380 of the ring engaging guiding track 1350. It is a particular feature of an embodiment of the present invention that since the priming portion 1380 is helical in its shape, the guidance of the tooth 1260 therealong urges slight axial forward displacement of the plunger assembly 1050.

It is specifically seen in FIG. 27G that following rotation of the ring assembly 1060 relative to the outer housing 1002, the radially inwardly extending fingers 1252 of snap portions 1250 of the ring portion 1062 are seated within yet another one of the recesses 1142 of the outer housing 1002 in this priming operative orientation, as compared to their position as shown in FIG. 24D. It is a particular feature of an embodiment of the present invention that this engagement between radially inwardly extending fingers 1252 and recesses 1142 prevents rotation of the ring assembly 1060 relative to the outer housing 1002 in an opposite rotational direction, preferably in a counter-clockwise direction.

It is specifically seen in FIGS. 27E and 27F that at the end of the priming operative orientation the tooth 1260 is disposed at the end of the helical priming portion 1380 and is aligned with the injection portion 1390 of the of the ring engaging guiding track 1350.

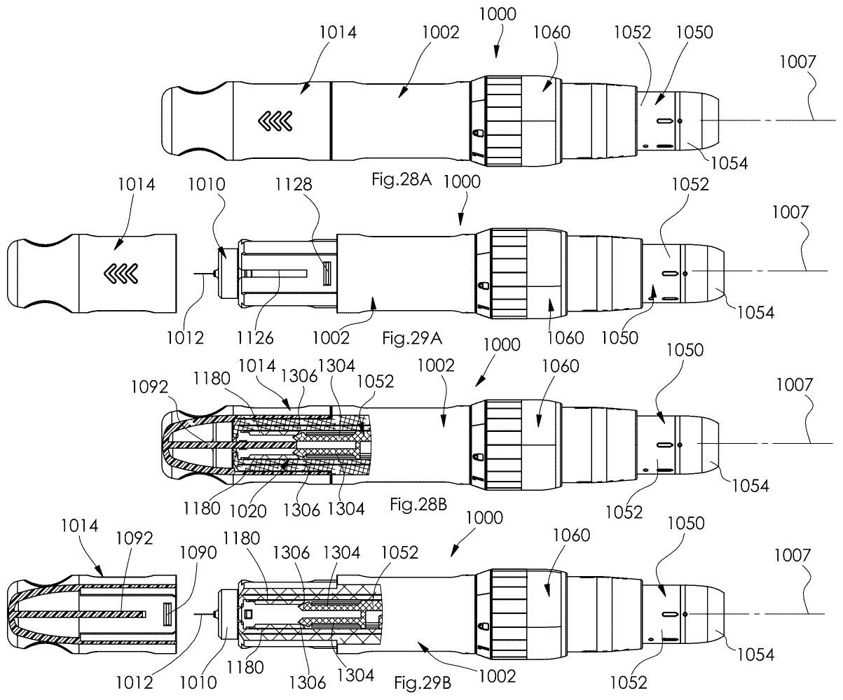

It is noted that at the end of this priming operative orientation of the dual chamber injection system 1000, the forward axial displacement of the plunger assembly 1050 relative to the outer housing 1002 is prevented due to the presence of the protective cap 1014 that is mounted over the forward end 1004 of the outer housing 1002 and is utilized to prevent inadvertent injection of medicament by premature displacement of the plunger assembly 1050 forwardly, as explained in detail hereinbelow with reference to FIGS. 28A-29B.

It is a particular feature of an embodiment of the present invention that forward axial displacement of the plunger assembly 1050 relative to the syringe holder 1020 is guided by means of engagement between snap portions 1200 of the syringe holder 1020 and syringe holder engaging guiding tracks 1450 of the plunger rod inner portion 1054.

Following relative axial displacement of the plunger assembly 1050 relative to the syringe holder 1020 in this priming operative orientation, as particularly seen in FIG. 27C, snap portions 1200 of the syringe holder 1020 are now more rearwardly disposed relative to the shaft portion 1440 of the plunger rod inner portion 1054, as compared to the illustrated in FIG. 26C. Specifically, radially inwardly directed teeth 1202 of snap portions 1200 of the syringe holder 1020 are now seated rearwardly of rearwardly tapered priming surface 1460, now engaging injection surface 1464 of syringe holder engaging guiding tracks 1450 of the plunger rod inner portion 1054.

Displacement of the plunger assembly 1050 rearwardly is prevented in this priming operative orientation due to engagement of the radially inwardly directed teeth 1202 with step 1456 of syringe holder engaging guiding tracks 1450 of the plunger rod inner portion 1054.

Reference is now made to FIGS. 28A and 28B, which are simplified drawings of the dual chamber injection system 1000 of FIGS. 14A-22D in a cap removal operative orientation shown before removal of the protective cap 1014, including respectively a simplified plan side view and a cut-out perspective view in FIG. 28B. Reference is additionally made to FIGS. 29A and 29B, which are simplified drawings of the dual chamber injection system 1000 of FIGS. 14A-22D in a cap removal operative orientation shown after removal of the protective cap 1014, including respectively a simplified plan side view and a cut-out perspective view in FIG. 29B.

It is seen that in this cap removal operative orientation the protective cap 1014 is detached from the outer housing 1002, thereby permitting further axial forward displacement of the plunger assembly 1050 in order to eject medication from the medicament cartridge 1040.

It is particularly seen in FIGS. 28A & 28B that the protective cap 1014 is mounted over the forward end 1004 of the outer housing 1002 and over the needle 1012, thereby protecting the needle 1012. The ribs 1092 of the protective cap 1014 are inserted into grooves 1126 of the outer housing 1002 when the protective cap 1014 is mounted over the outer housing 1002.

It is a particular feature of an embodiment of the present invention that further axial forward displacement of the plunger assembly 1050 at this end of priming operative orientation is prevented as long as the protective cap 1014 remains mounted onto the outer housing 1002. Specifically, at the end of priming operative orientation, described and illustrated hereinabove with reference to FIGS. 27A-27G, the lateral extensions 1306 of forwardly extending arms 1304 of the plunger rod outer portion 1502 are disposed adjacent the rearward ends of protrusions 1180 of the syringe holder 1020 and are supported thereon. Longitudinal ribs 1092 of the protective cap 1014 is inserted into grooves 1126 of the outer housing 1002 and the rearward ends of the ribs 1092 are disposed partially between the lateral extensions 1306 of the plunger rod outer portion 1052. The lateral extensions 1306 are at least partially enclosed between the rib 1092 of the protective cap 1014 and the protrusions 1180 of the syringe holder 1020 and thus the forwardly extending arms 1304 of the plunger rod outer portion 1502 are prevented from radial inward deflection thereof, thereby preventing forward axial displacement of the plunger assembly 1050 relative to the medicament cartridge 1040.

It is particularly seen in FIGS. 29A & 29B that the protective cap 1014 is detached from the outer housing 1002 and thus the lateral extensions 1306 are not enclosed between the rib 1092 of the protective cap 1014 and the protrusions 1180 of the syringe holder 1020 anymore, thereby allowing the forwardly extending arms 1304 to deflect radially inwardly in between protrusions 1180 of the syringe holder 1020 and thereby enable forward axial displacement of the plunger assembly 1050.

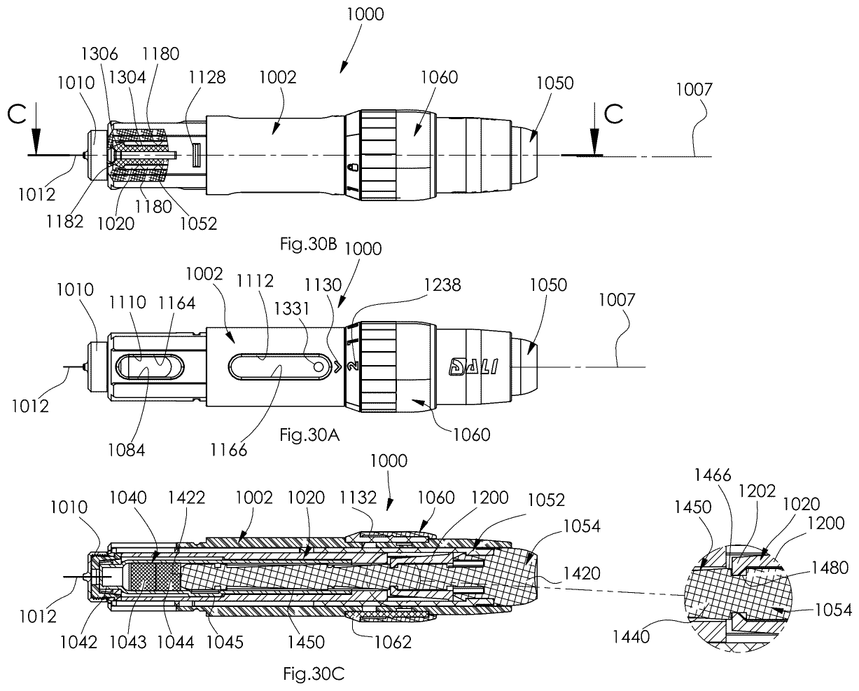

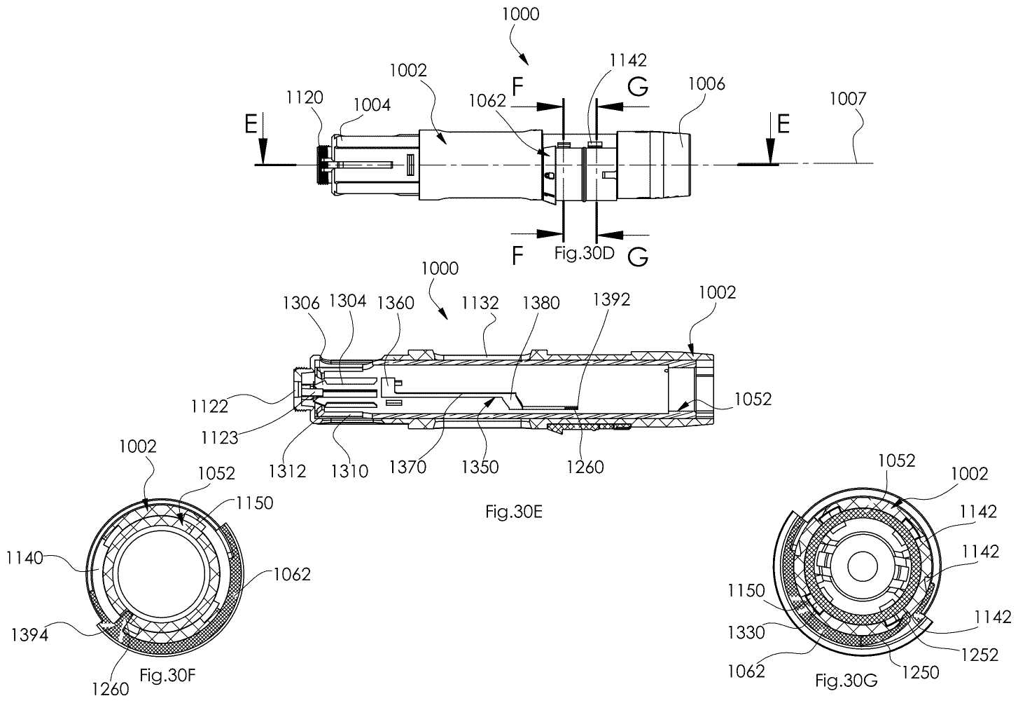

Reference is now made to FIGS. 30A-30C, which are simplified drawings of the dual chamber injection system 1000 of FIGS. 14A-22D in an end of injection operative orientation, including respectively two simplified different plan side views and a sectional view taken along lines C-C in FIG. 30B. Reference is additionally made to FIGS. 30D-30G, which are simplified drawings of the dual chamber injection system 1000 of FIGS. 14A-22D in an end of injection operative orientation, where only the outer housing 1002, the plunger rod outer portion 1052 and one ring portion 1062 is shown to clearly describe the interactions therebetween, including respectively a plan side view and three different sectional views taken along lines E-E, F-F and G-G in FIG. 30D.

The dual chamber injection system 1000 is seen in FIGS. 30A-30G in the end of injection operative orientation. It is seen particularly in FIGS. 30A-30C that the plunger assembly 1050 is fully forwardly axially displaced relative to the outer housing 1002 and syringe holder 1020.

It is seen that in this end of injection operative orientation that the pistons 1043 and 1044 are now forwardly axially displaced by the plunger assembly 1050 up to the forward end of the medicament cartridge 1040 to enable injection of the entire amount of medicament from the medicament cartridge 1040. The dual chamber injection system 1000 is now at the end of medicament delivery operative orientation and is ready for disposal.

It is noted that most of the spatial relationships between the different components of the dual chamber injection system 1000 remain the same as described and illustrated with respect to FIGS. 27A-27G, besides the following:

The protective cap 1014 is detached from the outer housing 1002 as described and illustrated in FIGS. 29A & 29B.

The plunger assembly 1050 is being axially forwardly displaced relative to the outer housing 1002 and the syringe holder 1020 up to alignment of demarcation 1331 provided on the outer surface of the plunger rod outer portion 1052, and visible through window 1112, with demarcation 1130 provided on the outer housing 1002, thereby indicating to the user that the entire amount of medicament was injected.

It is specifically seen in FIG. 30B that once the plunger rod outer portion 1052 has reached the end of injection position, lateral projections 1306 of arms 1304 return to their non-deflected orientation and are now seated adjacent the inward wall portions 1182 of the syringe holder 1020, thereby enabling re-attachment of the protective cap 1014 to the outer housing 1002, such that the rib 1092 of the protective cap 1014 is inserted in between the arms 1304 of the plunger rod outer portion 1052 to protect the needle 1012 after injection.

It is noted that in this end of injection operative orientation, the ring assembly 1060 remains in the same rotational orientation relative to the outer housing 1002 as illustrated in FIG. 27G. Thus, the orientation of the ring assembly 1060 relative to outer housing 1002 as illustrated in FIG. 30G remains preferably identical to the orientation of the ring assembly 1060 as illustrated in FIG. 27G.

It is specifically seen in FIGS. 30E and 30F that during injection, upon forward axial displacement of the plunger assembly 1050, the tooth 1260 is guided along the injection portion 1390 of the ring engaging guiding track 1350 and at the end of injection operative orientation the tooth 1260 is disposed at the end of the injection portion 1390, adjacent the rearward most edge 1392.

It is a particular feature of an embodiment of the present invention that forward axial displacement of the plunger assembly 1050 relative to the syringe holder 1020 is guided by means of engagement between snap portions 1200 of the syringe holder 1020 and syringe holder engaging guiding tracks 1450 of the plunger rod inner portion 1054.

Following relative axial displacement of the plunger assembly 1050 relative to the syringe holder 1020 in this end of injection operative orientation, as particularly seen in FIG. 30C, snap portions 1200 of the syringe holder 1020 are now more rearwardly disposed relative to the shaft portion 1440 of the plunger rod inner portion 1054, as compared to the illustrated in FIG. 27C. Specifically, radially inwardly directed teeth 1202 of snap portions 1200 of the syringe holder 1020 are guided along the syringe holder engaging guiding tracks 1450 of the plunger rod inner portion 1054 and upon full forward displacement of the plunger assembly 1050, the inwardly directed teeth 1202 of snap portions 1200 engage the locking gap 1480 of the syringe holder engaging guiding tracks 1450 of the plunger rod inner portion 1054.

It is a particular feature of an embodiment of the present invention that displacement of the plunger assembly 1050 rearwardly is prevented in this end of injection operative orientation due to the above-mentioned engagement of the snap portions of the syringe holder 1020 with the locking gap 1480 of the plunger rod inner portion 1054.

It will be appreciated by persons skilled in the art that the present invention is not limited by what has been particularly shown and described hereinabove. Rather the scope of the present invention includes both combinations and sub-combinations of various features described hereinabove as well as variations and modifications thereof which are not in the prior art.

1-76. (canceled)

77. A dual chamber injection system, comprising:
a guiding element arranged along a longitudinal axis and configured to at least partially contain a medicament container, said guiding element defines an inner surface and at least one first guiding feature formed thereon;
a plunger rod assembly, comprising a plunger outer portion arranged along said longitudinal axis and a plunger inner portion arranged concentrically within said plunger outer portion and having at least one second guiding feature arranged on an outer surface of said plunger inner portion;
said plunger outer portion is disposed externally relative to said guiding element; said plunger inner portion is at least partially received into said guiding element;
and wherein said plunger rod assembly is displaceable relative to said guiding element and said displacement is guided by engagement of said at least one first guiding feature with said at least second guiding feature.

78. The dual chamber injection system of claim 77, and wherein said first guiding feature is a protrusion extending radially inwardly from the inner surface of said guiding element and said second guiding feature is a guiding groove.

79. The dual chamber injection system of claim 77, and wherein said guiding element defines an outer surface and at least one arm extending therefrom and being radially deflectable upon application of pressure thereon; said plunger outer portion having a plurality of openings formed along the circumference thereof; and wherein an indication is provided to the user to indicate transitions between different operative orientations of said dual chamber injection system provided by engagement of said at least one arm with one of said plurality of openings.

80. The dual chamber injection system of claim 78, and wherein said at least one guiding groove is continuous and having portions thereof that are disposed in different axial orientations along said outer surface of said plunger inner portion.

81. The dual chamber injection system of claim 78, and wherein said at least one guiding groove is continuous and having portions thereof that are disposed in different angular orientations along said outer surface of said plunger inner portion.

82. The dual chamber injection system of claim 78, and wherein in a locked operative orientation of said dual chamber injection system said plunger rod assembly is prevented from axial displacement relative to said guiding element due to engagement of said at least one protrusion with a locking portion of said at least one guiding groove.

83. The dual chamber injection system of claim 77, and wherein said guiding element further comprises at least one locking arm; and said outer plunger portion has a rearwardly facing surface formed on said inner surface; and wherein in an end of injection operative orientation of said dual chamber injection system said plunger rod assembly is prevented from forward displacement relative to said guiding element due to engagement of said at least one locking arm with said rearwardly facing surface.

84. The dual chamber injection system of claim 2, and wherein said plunger rod assembly is rotatable relative to said guiding element in some operative orientations when said at least one protrusion of said guiding element engages a certain portion of said at least one guiding groove, and wherein said plunger rod assembly is axially displaceable relative to said guiding element in other operative orientations when said at least one protrusion of said guiding element engages another portion of said at least one guiding groove.

85. A dual chamber injection system, comprising:
a guiding element arranged along a longitudinal axis and configured to at least partially contain a medicament container, said guiding element defines an outer surface and at least one arm extending therefrom and being radially deflectable upon application of pressure thereon;
a plunger rod assembly, comprising a plunger outer portion arranged along said longitudinal axis and a plunger inner portion arranged concentrically within said plunger outer portion; said plunger outer portion having a plurality of openings formed along the circumference thereof;
said plunger outer portion is disposed externally relative to said guiding element; said plunger inner portion is at least partially received into said guiding element;
and wherein displacement of said plunger rod assembly relative to said guiding element in at least one direction is prevented due to engagement of said at least one arm of said guiding element with said one of said plurality of openings of said plunger outer portion.

86. The dual chamber injection system of claim 85, and wherein said guiding element defines an inner surface and at least one protrusion extending radially inwardly therefrom; said plunger inner portion having at least one guiding groove arranged on an outer surface of said plunger inner portion; and wherein said plunger rod assembly is displaceable relative to said guiding element and said displacement is guided by engagement of said at least one protrusion with said at least one guiding groove.

87. The dual chamber injection system of claim 86, and wherein in a locked operative orientation of said dual chamber injection system said plunger rod assembly is prevented from axial displacement relative to said guiding element due to engagement of said at least one protrusion with a locking portion of said at least one guiding groove.

88. The dual chamber injection system of claim 85, and wherein said guiding element further comprises at least one locking arm; and said outer plunger portion has a rearwardly facing surface formed on said inner surface; and wherein in an end of injection operative orientation of said dual chamber injection system said plunger rod assembly is prevented from rearward displacement relative to said guiding element due to engagement of said at least one locking arm with said rearwardly facing surface.

89. The dual chamber injection system of claim 85, and wherein rearward axial displacement of said plunger rod assembly relative to said guiding element is prevented due to engagement of said at least one arm of said guiding element with said one of said plurality of openings of said plunger outer portion.

90. The dual chamber injection system of claim 85, and wherein rotational displacement of said plunger rod assembly relative to said guiding element is prevented in at least one rotational direction due to engagement of said at least one arm of said guiding element with said one of said plurality of openings of said plunger outer portion.

91. The dual chamber injection system of claim 85, and wherein an indication is provided to the user to indicate transitions between different operative orientations of said dual chamber injection system provided by engagement of said at least one arm with one of said plurality of openings.

92. A dual chamber injection system, comprising:
a housing element arranged along a longitudinal axis and configured to at least partially contain a medicament container;
a plunger rod assembly, comprising a plunger outer portion arranged along said longitudinal axis and a plunger inner portion arranged concentrically within said plunger outer portion and having at least one guiding groove arranged on said plunger outer portion;
said plunger rod assembly is at least partially received into said housing element;
a rotatable ring assembly axially fixedly mounted onto a portion of said housing element and having a radially inwardly extending protrusion engageable with a portion of said at least one guiding groove, whereas displacement of said radially inwardly extending protrusion along said guiding groove enables transition of said dual chamber injection system between the different operative orientations thereof.

93. The dual chamber injection system according to claim 92, also comprising a guiding element being at least partially received into said housing element and configured to at least partially contain said medicament container having a septum at a forward end thereof.

94. The dual chamber injection system according to claim 92, wherein said ring assembly is engageable with said plunger outer portion, whereas axial displacement of said plunger rod assembly relative to said housing element is permitted upon rotation of said ring assembly relative to said housing element, thereby enabling transition of said dual chamber injection system between at least some of the different operative orientations thereof.

95. The dual chamber injection system according to claim 92, wherein said at least one guiding groove comprises a locking portion, a reconstitution portion, a helical priming portion and an injection portion, wherein said reconstitution portion is radially and axially offset from said injection portion and said locking portion is disposed generally transversely with respect to said reconstitution portion.

96. The dual chamber injection system according to claim 92, wherein a needle is configured to be fixedly attached to said housing element and a protective cap removably attached to said housing element and configured to protect said needle in certain operative orientations of said dual chamber injection system; and wherein said plunger rod assembly is prevented from forward axial displacement relative to said housing element beyond a certain point up to removal of said protective cap from said housing element.
